SEQUENCE LISTING (7 sequences)

SEQ ID NO: 1 - DNA, 8341 bp, Artificial sequence
  Synthetic construct
  misc_feature: eTIP_DI-delta746-Venus-3355
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctccagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctttacaagcccctctccaccaaggatctgaccacatatggattcggacaccaaaacaaagcggtgtacactgcaggttacaaaatttgcaactaccacttggccactcaggatgatttgcaaaacgcagtgaacgtcatgtggagtagagacctcttagtcacagaatcaagagcccagggcaccgattcaatcgcaaggtgcaattgcaacgcaggggtgtactactgcgagtctagaaggaaatactacccagtatccttcgttggcccaacgttccagtacatggaggctaataactattacccagctaggtaccagtcccatatgctcattggccatggattcgcatctccaggggattgtggtggcatactcagatgtcaccacggggtgatagggatcattactgctggtggcgaagggttggttgcattttcagacattagagacttgtatgcctacgaagaagaagccatggaacaaggcatcaccaattacatagagtcacttggggccgcatttggaagtggatttactcagcagattagcgacaaaataacagagttgaccaatatggtgaccagtaccatcactgaaaagctacttaagaacttgatcaagatcatatcctcactagttattataactaggaactatgaagacaccacaacagtgctcgctaccctggcccttcttgggtgtgatgcttcaccatggcagtggcttagaaagaaagcatgcgatgttctggagataccttatgtcatcaagcaaggtgacagttggttgaagaagtttactgaagcatgcaacgcagctaagggactggagtgggtgtcaaacaaaatctcaaaattcattgattggctcaaggagaaaattatcccacaagctagagataagttggaatttgtaacaaaacttagacaactagaaatgctggaaaaccaaatctcaactatacaccaatcatgccctagtcaggaacaccaggaaattctattcaataatgtcagatggttatccatccagtctaagaggtttgcccctctttacgcagtggaagccaaaagaatacagaaactagagcatactattaacaactacatacagttcaagagcaaacaccgtattgaaccagtatgtttgctagtacatggcagccccggaacaggtaaatctgtagcaaccaacctgattgctagagccatagctgaaagagaaaacacgtccacgtactcgctacccccggatccatcacacttcgacggatacaaacaacagggagtggtgattatggacgacctgaatcaaaacccagatggtgcggacatgaagctgttctgtcagatggtatcaacagtggagtttataccacccatggcatccctggaggagaaaggaatcctgtttacttcaaattacgttctagcatccacaaactcaagcagaatttccccccccactgtggcacacagtgatgcattagccaggcgctttgcgttcgacatggacattcaggtcatgaatgagtattctagagatgggaaattgaacatggccatggctactgaaatgtgtaagaactgtcaccaaccagcaaactttaagagatgctgtcctttagtgtgtggtaaggcaattcaattaatggacaaatcttccagagttagatacagtattgaccagatcactacaatgattatcaatgagagaaacagaagatccaacattggcaattgtatggaggctttgtttcaaggaccactccagtataaagacttgaaaattgacatcaagacgagtccccctcctgaatgtatcaatgacttgctccaagcagttgactcccaggaggtgagagattactgtgagaagaagggttggatagtcaacatcaccagccaggttcaaacagaaaggaacatcaacagggcaatgacaattctacaagcggtgacaaccttcgccgcagtggctggagttgtctatgtcatgtataaactgtttgctggacaccagggagcatacactggtttaccaaacaaaaaacccaacgtgcccaccattcggacagcaaaggtacaaggaccagggttcgattacgcagtggctatggctaaaagaaacattgttacagcaactactagcaagggagagttcactatgttaggagtccacgacaacgtggctattttaccaacccacgcttcacctggtgaaagcattgtgatcgatggcaaagaagtggagatcttggatgccaaagcgctcgaagatcaagcaggaaccaatcttgaaatcactataatcactctaaagagaaatgaaaagttcagagacattagaccacatatacctactcaaatcactgagacaaatgatggagtcttgatcgtgaacactagcaagtaccccaatatgtatgttcctgtcggtgctgtgactgaacagggatatctaaatctcggtgggcgccaaactgctcgtactctaatgtacaactttccaaccagagcaggacagtgtggtggagtcatcacatgtactgggaaagtcatcgggatgcatgttggtgggaacggttcacacgggtttgcagcggccctgaagcgatcatacttcactcagagtcaaggtgaaatccagtggatgagaccttcgaaggaagtgggatatccaatcataaatgccccgtccaaaaccaagcttgaacccagtgctttccactatgtgtttgaaggggtgaaggaaccagcagtcctcactaaaaacgatcccaggcttaagacagactttgaggaggcaattttctccaagtacgtgggtaacaaaattactgaagtggatgagtacatgaaagaggcagtagaccactatgctggccagctcatgtcactagacatcaacacagaacaaatgtgcttggaggatgccatgtatggcactgatggtctagaagcacttgatttgtccaccagtgctggctacccttatgtagcaatgggaaagaagaagagagacatcttgaacaaacaaaccagagacactaaggaaatgcaaaaactgctcgacacatatggaatcaacctcccactggtgacttatgtaaaggatgaacttagatccaaaacaaaggttgagcaggggaaatccagattaattgaagcttctagtttgaatgactcagtggcaatgagaatggcttttgggaacctatatgctgcttttcacaaaaacccaggagtgataacaggttcagcagtggggtgcgatccagatttgttttggagcaaaattccggtattgatggaagagaagctgtttgcttttgactacacagggtatgatgcatctctcagccctgcttggttcgaggcactaaagatggtgcttgagaaaatcggattcggagacagagttgactacatcgactacctaaaccactcacaccacctgtacaagaataaaacatactgtgtcaagggcggtatgccatctggctgctcaggcacttcaatttttaactcaatgattaacaacttgattatcaggacactcttactgaaaacctacaagggcatagatttagaccacctaaaaatgattgcctatggtgatgatgtaattgcttcctacccccatgaagttgacgctagtctcctagcccaatcaggaaaagactatggactaactatgactccagctgacaaatcagctacatttgaaacagtcacatgggagaatgtaacattcttgaagagattcttcagggcagacgagaaatacccatttcttattcatccagtaatgccaatgaaggaaattcatgaatcaattagatggactaaagatcctaggaacactcaggatcacgttcgctctctgtgccttttagcttggcacaatggcgaagaagaatataacaaattcctagctaaaatcaggagtgtgccaattggaagagctttattgctcccagagtactcaacattgtaccgccgttggcttgactcattttagtaaccctacctcagtcgaattggattgggtcatactgttgtaggggtaaatttttctttaattcggagaaaaaaaaaaaaaaaaaaaaaaaaaaaaaaaaaaaaaaaaaaaaaaaaaaaaaaaaaaaaaaaaaaaaaaaaaaagggcccgaattcatcgatgaattaattcttgaagacgaaagggcctcgtgatacgcctatttttataggttaatgtcatgataataatggtttcttagactacgcgtagtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaatacgactcactatagggtgttttaactgatgaggccgaaaggccgaaaacccggtatcccgggttc

SEQ ID NO: 2 - DNA, 22 bp, Artificial sequence
  Synthetic construct
  misc_feature: PV1 primer
ccacatacagacgatcccatac

SEQ ID NO: 3 - DNA, 21 bp, Artificial sequence
  Synthetic construct
  misc_feature: PV1 primer
ctgcccagtgtgtgtagtaat

SEQ ID NO: 4 - DNA, 24 bp, Artificial sequence
  Synthetic construct
  misc_feature: PV1 primer
tctgcctgtcactctctccagctt

SEQ ID NO: 5 - DNA, 18 bp, Artificial sequence
  Synthetic construct
gacagcgaagccaatcca

SEQ ID NO: 6 - DNA, 21 bp, Artificial sequence
  Synthetic construct
  misc_feature: PV1 primer
ccatgtgtagtcgtcccattt

SEQ ID NO: 7 - DNA, 24 bp, Artificial sequence
  Synthetic construct
  misc_feature: PV1 primer
acgaaagagtcggtaccaccaggc

(19) United States
(12) Patent Application Publication
(10) Pub. No.: US 2023/0233669 A1
(43) Pub. Date: Jul. 27, 2023

(54) RECOMBINANT ENTEROVIRUSES AND USES THEREOF

(71) Applicants: The Regents of the University of California, Oakland, CA (US); Aleph Therapeutics, Inc., Stanford, CA (US)

(72) Inventors: Raul ANDINO-PAVLOVSKY, San Francisco, CA (US); Yinghong XIAO, San Francisco, CA (US); Gilad DOITSH, San Francisco, CA (US); Robert NAKAMURA, Stanford, CA (US); Dale TALBOT, Stanford, CA (US)

(21) Appl. No.: 18/003,799

(22) PCT Filed: Jul. 2, 2021
(86) PCT No.: PCT/US2021/040237

§ 371 Date: Dec. 29, 2022

Related U.S. Application Data
(60) Provisional application No. 63/047,398, filed on Jul. 2, 2020.

(51) Int. Cl. A61K 39/215; C12N 7/00; A61K 39/145; A61K 39/155; A61K 39/125; A61K 39/39; A61P 31/14

(52) U.S. Cl. CPC A61K 39/215; C12N 7/00; A61K 39/145; A61K 39/155; A61K 39/125; A61K 39/39; A61P 31/14; C12N 2770/32634; C12N 2770/32621; C12N 2770/32652; C12N 2770/32671; C12N 2770/20034; C12N 2770/20022; A61K 2039/5256

(57) ABSTRACT

The present disclosure generally relates to, inter alia, to nucleic acid constructs encoding a modified enterovirus genome that is devoid of partial or complete nucleic acid sequences encoding viral structural proteins. The disclosure also provides compositions and methods useful for producing defective interfering particles (DIPs) of enteroviruses, and for the prevention and/or treatment of various health conditions such as immune diseases and viral infections.

### CROSS-REFERENCE TO RELATED APPLICATION

The present application claims priority to U.S. Provisional Patent Application Ser. No. 63/047,398, filed on Jul. 2, 2020, the disclosure of which is incorporated by reference herein in its entirety, including any drawings.

### STATEMENT REGARDING FEDERALLY SPONSORED R&D

This invention was made with government support under grant no. HR0011-17-2-0027 awarded by The Defense Advanced Research Project Agency. The government has certain rights in the invention.

### INCORPORATION OF THE SEQUENCE LISTING

The material in the accompanying Sequence Listing is hereby incorporated by reference into this application. The accompanying Sequence Listing text file, named 048536_687001WO_Sequence Listing_ST25.txt, was created on Jun. 25, 2021 and is 13 KB.

### FIELD

The present disclosure relates generally to the field of molecular virology and immunology, and particularly relates to nucleic acid constructs encoding a modified enterovirus genome that is devoid at least a portion of the sequence encoding viral structural proteins. The disclosure also provides compositions and methods useful for producing defective interfering particles (DIPs) of enteroviruses, and for the prevention and/or treatment of various health conditions such as immune diseases and viral infections.

### BACKGROUND

Viruses are a major threat to human and animal health and significant efforts have been made over the last century to prevent and treat viral diseases. As a result, several potent antivirals are currently available for the treatment of diseases associated with various viruses such as human immunodeficiency viruses (HIV), influenza A viruses, and herpes viruses. In recent years, significant progress has been made in developing potent, safe, and affordable viral human and veterinary vaccines using egg- and cell-based production systems. In addition, several novel approaches toward vaccination, including DNA and RNA vaccines, are under development.

Despite these many advances, significant challenges remain. For example, effectively controlling the spread of viral infectious diseases has proven very challenging. On the one hand, humans are constantly challenged by new viruses including Zika virus, MERS-coronavirus, and Ebola virus. In particular, recent outbreaks of the enterovirus D68(EV-D68), flu, and COVID-19 have raised health concern in the U.S. and worldwide. On the other hand, most viruses change rapidly in the face of selective pressure, and emergence of antiviral drug resistance is of major concern. These challenges can only partly be addressed by existing technologies, since the development of new vaccines, the adaptation of existing manufacturing processes, the identification of new antiviral targets, and the development of new antivirals are often time consuming and costly processes.

Thus, additional safe and effective approaches are still urgently needed for the prevention, treatment, and/or management of health conditions associated with viral infections.

### SUMMARY

The present disclosure relates generally to the development of immuno-therapeutics, such as enteroviral defective interfering (DI) particles (DIPs) and pharmaceutical compositions comprising the same for use in the prevention and management of various health conditions such as immune diseases and microbial infection. In particular, as described in greater detail herein, the absence of at least a portion of the nucleic acid sequence encoding viral structural proteins was found necessary and sufficient for the DI phenotype. In addition, as illustrated in the Examples below, the virus-virus interactions between the populations of wild-type viral particles and DI particles could provide a way of quantitative modulation of immune targets in virus-host interactions in pathogenesis and persistence of viral infection. In particular, as illustrated in Examples 12-13, enteroviral DIPs can be used as a broad spectrum antiviral against many viruses, including poliovirus, non-polio enterovirus, rhinovirus and influenza virus. In particular, the data presented herein demonstrate that enteroviral DIPs can provide protection against the lethal poliovirus at the mucosal surface by intranasal infection as prophylactic administration and/or therapeutic administration. Furthermore, the data presented herein demonstrate that DIP-induced mucosal immunity in respiratory tract contributes to the protection effect.

In one aspect of the disclosure, provided herein are nucleic acid constructs including a nucleic acid sequence encoding a modified enterovirus genome, wherein the modified enterovirus genome is devoid of at least a portion of the nucleic acid sequence encoding viral structural proteins.

Non-limiting exemplary embodiments of the nucleic acid constructs of the disclosure can include one or more of the following features. In some embodiments, the modified enterovirus genome is devoid of at least a portion of the sequence encoding VP1, VP2, VP3, VP4, or a combination of any thereof. In some embodiments, the modified enterovirus genome or replicon RNA is devoid of a substantial portion of the nucleic acid sequence encoding viral structural proteins. In some embodiments, the modified enterovirus genome comprises no nucleic acid sequence encoding viral structural proteins. In some embodiments, the modified enterovirus genome is derived from a virus belonging to a Rhinovirus species selected from the group consisting of Rhinovirus A, Rhinovirus B, and Rhinovirus C. In some embodiments, the modified enterovirus genome is derived from a virus belonging to an Enterovirus species selected from the group consisting of Enterovirus A, Enterovirus B, Enterovirus C, Enterovirus D, Enterovirus E, Enterovirus F, Enterovirus G, Enterovirus H, Enterovirus I, Enterovirus J, Enterovirus K, and Enterovirus L. In some embodiments, the modified enterovirus genome is derived from a poliovirus of the Enterovirus C species. In some embodiments, the modified enterovirus genome is derived from a poliovirus serotype selected from the group consisting of PV1, PV2, and PV3. In some embodiments, the modified poliovirus genome or replicon RNA is derived from poliovirus type 1 (PV1). In some embodiments, the nucleic acid sequence encoding the modified poliovirus genome has at least 80%, at least 90%, at least 95%, at least 96%, at least 97%, at least 98%, at least 99%, or 100% sequence identity to the nucleic acid sequence of SEQ ID NO: 1.

In some embodiments of the disclosure, the nucleic acid sequence encoding a modified poliovirus genome is operably linked to a heterologous nucleic acid sequence. In some embodiments, the heterologous nucleic acid sequence comprises a promoter sequence or a coding sequence for a selectable marker. In some embodiments, the nucleic acid sequence encoding a modified poliovirus genome is incorporated into an expression cassette or an expression vector.

In one aspect, some embodiments of the disclosure relate to defective interfering (DI) particles (DIPs) of enterovirus. In some embodiments, the enteroviral DI particles include a nucleic acid construct of the disclosure. In some embodiments, the enteroviral DI particles include a nucleic acid construct of the disclosure, which is encapsidated by heterologous capsid structural proteins.

In another aspect, provided herein are recombinant cells including (a) a nucleic acid construct of the disclosure, and/or (b) a DI particle of the disclosure. Non-limiting exemplary embodiments of the recombinant cells of the disclosure can include one or more of the following features. In some embodiments, the recombinant cell is a eukaryotic cell. In some embodiments, the eukaryotic cell is an animal cell. In some embodiments, the animal cell is a human cell.

In another aspect, provided herein are methods for producing a defective interfering (DI) particle of enterovirus, the methods include: a) providing a host cell engineered to express enterovirus structural proteins or portions thereof, b) transfecting the provided host cell with a nucleic acid construct of the disclosure; and c) culturing the transfected host cell under conditions for production of a DIP of enterovirus comprising the nucleic acid construct encapsidated by the expressed enterovirus structural proteins or portion thereof. In some embodiments, the methods for producing enteroviral defective interfering (DI) particles described herein further include harvesting the produced DIP. Accordingly, also provided herein are defective interfering (DI) particles produced by the methods described herein.

In yet another aspect, provided herein are pharmaceutical compositions including a pharmaceutically acceptable excipient and (a) a DIP of the disclosure; (b) a nucleic acid construct of the disclosure; and/or (c) a recombinant cell of the disclosure.

Non-limiting exemplary embodiments of the pharmaceutical compositions of the disclosure can include one or more of the following features. In some embodiments, the composition comprises a DIP of the disclosure and a pharmaceutically acceptable excipient. In some embodiments, the composition comprises a nucleic acid construct of the disclosure and a pharmaceutically acceptable excipient. In some embodiments, the composition is formulated in a liposome, a lipid nanoparticle, or a polymer nanoparticle. In some embodiments, the composition is an immunogenic composition. In some embodiments, the immunogenic composition is formulated as a vaccine. In some embodiments, the pharmaceutical composition is formulated as an adjuvant. In some embodiments, the pharmaceutical composition is formulated for one or more of intranasal administration, transdermal administration, intraperitoneal administration, intramuscular administration, intravenous administration, and oral administration.

In another aspect, provided herein are methods for eliciting an immune response in a subject in need thereof, the method comprises administering to the subject a composition including: (a) a DIP of the disclosure; (b) a nucleic acid construct of the disclosure; (c) a recombinant cell of the disclosure; and/or (d) a pharmaceutical composition of the disclosure.

In yet another aspect, provided herein are methods for preventing and/or treating a health condition in a subject in need thereof, the methods include prophylactically or therapeutically administering to the subject a composition including: (a) a DIP of the disclosure; (b) a nucleic acid construct of the disclosure; (c) a recombinant cell of the disclosure; and/or (d) a pharmaceutical composition of the disclosure.

Non-limiting exemplary embodiments of the methods of the disclosure can include one or more of the following features. In some embodiments, the condition is an immune disease or an infection. In some embodiments, the subject has or is suspected of having a condition associated with an immune disease or an infection. In some embodiments, the infection is a seasonal respiratory viral infection or an acute respiratory viral infection. In some embodiments, the infection is caused by a virus belonging to a species of the Human orthopneumovirus genus, a species of the Enterovirus family, a species of the Coronaviridae family, or a subtype of the Orthomyxoviridae family. In some embodiments, the orthomyxovirus is an influenza A virus or a Parainfluenza virus. In some embodiments, the influenza A virus is selected from the group consisting of subtypes H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7. In some embodiments, the parainfluenza virus is selected from the group consisting of subtypes HPIV-1, HPIV-2, HPIV-3, and HPIV-4. In some embodiments, the coronavirus is β-CoV severe acute respiratory syndrome coronavirus (SARS-CoV), severe acute respiratory syndrome coronavirus 2 (SARS-CoV-2). In some embodiments, the coronavirus β-CoV infection is associated with one or more subgenus Sarbecovirus selected from the group consisting of severe acute respiratory syndrome coronavirus SARSr-CoV (which includes all its strains such as SARS-CoV, SARS-CoV-2, and Bat SL-CoV-WIV1), subgenus Merbecovirus consisting of Tylonycteris bat coronavirus HKU4 (BtCoV-HKU4), Pipistrellus bat coronavirus HKU5 (BtCoV-HKU5), and Middle East respiratory syndrome-related coronavirus MERS-CoV (which includes the species HCoV-229E, HCoV-NL63, HCoV-OC43, HCoV-HKU1). In some embodiments, the human orthomyxovirus is a human respiratory syncytial virus (HRSV). In some embodiments, the HRSV is associated with subtype A and/or subtype B.

In some embodiments of the disclosure, the viral infection is an enteroviral infection or a rhinoviral infection. In some embodiments, the rhinoviral infection is associated with one or more Rhinovirus species selected from the group consisting of rhinovirus A species, rhinovirus B species, and rhinovirus C species. In some embodiments, the enteroviral infection is associated with one or more Enterovirus species selected from the group consisting of Enterovirus A species, Enterovirus B species, Enterovirus C species, Enterovirus D species, Enterovirus E species, Enterovirus F species, Enterovirus G species, Enterovirus H species, Enterovirus I species, Enterovirus J species, Enterovirus K species, and Enterovirus L species. In some embodiments, the viral infection is associated with one or more of poliovirus type 1 (PV1), poliovirus type 3 (PV3), coxsackievirus A2, coxsackievirus A4, coxsackievirus A16, coxsackievirus B1, coxsackievirus B3 (CV-B3), coxsackievirus B6, Parechovirus (echovirus), enterovirus A71 (EV-A71), enterovirus D68 (EV-D68), rhinovirus HRV16, and rhinovirus HRV1B.

In some embodiments, the composition is formulated for one or more of intranasal administration, transdermal administration, intramuscular administration, intravenous administration, intraperitoneal administration, oral administration, or intra-cranial administration.

In some embodiments, the administered composition results in an increased production of interferon in the subject. In some embodiments, the composition is administered to the subject individually as a single therapy (monotherapy) or as a first therapy in combination with at least one additional therapies. In some embodiments, the at least one additional therapies is selected from the group consisting of chemotherapy, radiotherapy, immunotherapy, hormonal therapy, toxin therapy, targeted therapy, and surgery.

In another aspect, provided herein are kits eliciting an immune response, for the prevention, and/or for the treatment of a health condition or a viral infection, the kits including: (a) a DIP of the disclosure; (b) a nucleic acid construct of the disclosure; and/or (c) a pharmaceutical composition of the disclosure.

Each of the aspects and embodiments described herein are capable of being used together, unless excluded either explicitly or clearly from the context of the embodiment or aspect.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1E schematically summarize the results of experiments performed to illustrate an exemplary design of poliovirus DIPs and the production of eTIP in accordance with some embodiments of the disclosure.

FIG. 1A is a schematic of the wild-type poliovirus (PV) and eTIP genome. The PV genome includes a highly structured 5′UTR followed by a single open reading frame and a polyadenylated 3′UTR. The coding region can be separated into structural and non-structural genes. The structural genes (P1/Capsid) are required to form the viral capsid and the non-structural genes (P2/P3) are all required for a successful replication cycle. The defective interfering particle of poliovirus (eTIP): eTIP was lacking the P1 genes of PV1 virus and was replaced by GFP-Venus gene. eTIP2, which was a negative control, and was identical to eTIP1 except that it carried a deletion expanding from nucleotide 5,600 to 7,500 (the entire 3′ end of the genome). eTIP2 could not replicate but expressed most eTIP1 proteins.

FIG. 1B is a schematic illustrating the production of eTIP. Wild-type (WT) RNAs were transfected into packaging cells line, Hela S3 cells stable expression P1 genes (Hela S3/P1) of the poliovirus (Method). And the eTIP was titered in Hela S3 cells.

FIG. 1C pictorially summarizes the experimental results, with a silver stained SDS-polyacrylamide gel (left panel) and electron microscopy (EM) stain (right panel), illustrating a purification of the eTIP. In these experiments, eTIP1 were purified by sucrose gradient and examined by SDS-polyacrylamide gel electrophoresis with silver staining and electron microscopy and negative staining.

FIG. 1D schematically summarizes the results of experiments performed to illustrate co-infection in Hela cells, PV1, CVB3, PV3 and Rhinovirus (HRV16, HRViB) at M.O.I.=0.1. Virus+eTIP ratio was 1:1, 1:10, 1:20, 1:50 or 1:100. Samples were collected at 9 hours post-infection. Virus titer was measured by plaque assay. PV1 represents wild-type poliovirus type 1 mahoney strain. PV3 represents WT type 3 virus Leon strain. CVB3 represents wild-type coxsackievirus B3 strain.

FIG. 1E schematically summarizes the results of experiments performed to illustrate that eTIP inhibits Rhinovirus HRV87(EV-D68) infection in cell culture by co-infection. EV-D68 represents wild-type enterovirus D68, it also terms as HRV87.

FIGS. 2A-2H schematically summarize the results of experiments performed to illustrate that co-infected wild-type enteroviruses with eTIP increase the survive rate of infected animals.

FIG. 2A schematically summarizes the results of experiments performed to illustrate that co-infected wild-type enteroviruses with eTIP increase the survive rate of infected animals. In these experiments, 6 to 8-weeks-old Tg21 PVR interferon α/β receptor knockout (IFNAR^{-/-}) mice were infected with wild-type poliovirus (PV1) or with co-infected mixed PV1+eTIP by intramuscular (I.M.) route. 200 plaque forming units (PFU) PV1 alone or mixed PV1+eTIP at ratio 1:5000. The number of mice is between 13 and 14 per group (n=13-14). Solid line represents wild-type enteroviruses alone. Dash line represents co-infected group. The comparison of survival curve is performed by Log-rank (Mantel-Cox) test, p<0.05 as significant.

FIG. 2B is a graph illustrating tissue distribution in muscle (in plaque assay). IFNAR^{-/-} mice were infected with 200 PFU wild-type poliovirus or co-infected with mixed PV1+eTIP at ratio 1:5000 by intramuscular (I.M.) route. Tissues were collected as indicated time points (X-axis). Y-axis represent as PFU per gram tissue. Black bar represents PV1 titer in PV1 group. Grey bar represents PV1 titer in co-infection group.

FIG. 2C is a graph illustrating tissue distribution in spleen (in plaque assay). IFNAR^{-/-} mice were infected with 200 PFU wild-type poliovirus or co-infected with mixed PV1+eTIP at ratio 1:5000 by intramuscular (I.M.) route. Tissues were collected as indicated time points (X-axis). Y-axis represent as PFU per gram tissue. Black bar represents PV1 titer in PV1 group. Grey bar represents PV1 titer in co-infection group.

FIG. 2D is a graph illustrating tissue distribution in spinal cord (in plaque assay). IFNAR^{-/-} mice were infected with 200 PFU wild-type poliovirus or co-infected with mixed PV1+eTIP at ratio 1:5000 by intramuscular (I.M.) route. Tissues were collected as indicated time points (X-axis). Y-axis represent as PFU per gram tissue. Black bar represents PV1 titer in PV1 group. Grey bar represents PV1 titer in co-infection group. Compared with wild-type group, PV1 titer in spinal cord and brain of co-infection group was significantly reduced at 6 days post-infection.

FIG. 2E is a graph illustrating tissue distribution in brain (in plaque assay). IFNAR^{-/-} mice were infected with 200 PFU wild-type poliovirus or co-infected with mixed PV1+eTIP at ratio 1:5000 by intramuscular (I.M.) route. Tissues were collected as indicated time points (X-axis). Y-axis represent as PFU per gram tissue. Black bar represents PV1 titer in PV1 group. Grey bar represents PV1 titer in co-infection group. Compared with wild-type group, PV1 titer in spinal cord and brain of co-infection group was significantly reduced at 6 days post-infection.

FIG. 2F schematically summarizes the results of qPCR experiments performed to quantify viral genome in muscle. RNA genome copies were measured by digital droplet PCR instead of virus titer. Y-axis represents RNA genome copies per 1 mg total RNA. Solid line with triangle represents PV1 genome copies in wild-type virus alone group. Dashed line with dots represents PV1 genome copies in titer in co-infection group. Dash line with open squares represents eTIP genome copies in titer in co-infection group. The number of mice is three for each group and each time point (n=3). Two tails multiple-t-tests, P<0.05 represents as significant.

FIG. 2G schematically summarizes the results of qPCR experiments performed to quantify viral genome in spleen. RNA genome copies were measured by digital droplet PCR instead of virus titer. Y-axis represents RNA genome copies per 1 mg total RNA. Solid line with triangle represents PV1 genome copies in wild-type virus alone group. Dashed line with dots represents PV1 genome copies in titer in co-infection group. Dash line with open squares represents eTIP genome copies in titer in co-infection group. The number of mice is three for each group and each time point (n=3). Two tails multiple-t-tests, P<0.05 represents as significant.

FIG. 2H schematically summarizes the results of qPCR experiments performed to quantify viral genome in spinal cord. RNA genome copies were measured by digital droplet PCR instead of virus titer. Y-axis represents RNA genome copies per 1 mg total RNA. Solid line with triangle represents PV1 genome copies in wild-type virus alone group. Dashed line with dots represents PV1 genome copies in titer in co-infection group. Dash line with open squares represents eTIP genome copies in titer in co-infection group. The number of mice is three for each group and each time point (n=3). Two tails multiple-t-tests, P<0.05 represents as significant.

FIGS. 3A-3F schematically summarize the results of experiments performed to illustrate different ratios effect on eTIP protection against PV1. Co-infected wild-type enteroviruses with eTIP increase the survive rate of infected animals.

FIG. 3A is a graph illustrating survival rate when infected with 5000 PFU PV1 alone or mixed PV1+eTIP at ratio 1:10 and 1:100 (n=8-10). PV1 represents wild-type poliovirus type 1 mahoney strain. Tg21 PVR interferon α/β receptor knockout (IFNAR^{-/-}) mice were infected with wild-type (WT) enteroviruses alone or with co-infected mixed WT+eTIP by intramuscular (I.M.) route. Solid line with dot represents wild-type enteroviruses alone. Dash line with solid squares represents co-infected group.

FIG. 3B is a graph illustrating survival rate when infected with 5000 PFU PV3 alone or mixed PV3+eTIP at ratio 1:1000 (n=20). PV3 represents WT type 3 virus Leon strain. Tg21 PVR interferon α/β receptor knockout (IFNAR^{-/-}) mice were infected with wild-type (WT) enteroviruses alone or with co-infected mixed WT+eTIP by intramuscular (I.M.) route. Solid line with dot represents wild-type enteroviruses alone. Dash line with solid squares represents co-infected group.

FIG. 3C is a graph illustrating survival rate when infected with 20 PFU CVB3 alone or mixed CVB3+eTIP at ratio 1:50000 (n=20). CVB3 represents wild-type coxsackievirus B3 strain. Tg21 PVR interferon α/β receptor knockout (IFNAR^{-/-}) mice were infected with wild-type (WT) enteroviruses alone or with co-infected mixed WT+eTIP by intramuscular (I.M.) route. Solid line with dot represents wild-type enteroviruses alone. Dash line with solid squares represents co-infected group.

FIG. 3D is a graph illustrating survival rate when infected with 2×10³ TCID50 PFU EV-D68 alone or mixed EV-D68+eTIP at ratio 1:1000, 5-7 days-old pups infected by intra-cranical route. EV-D68 represents wild-type enterovirus D68. The number of mice is between 7 and 10 per group (n=7-10). Tg21 PVR interferon α/β receptor knockout (IFNAR^{-/-}) mice were infected with wild-type (WT) enteroviruses alone or with co-infected mixed WT+eTIP by intramuscular (I.M.) route. Solid line with dot represents wild-type enteroviruses alone. Dash line with solid squares represents co-infected group.

FIG. 3E schematically summarizes the results of an eTIP safety test in IFNAR^{-/-} mice. The comparison of survival curve is performed by Log-rank (Mantel-Cox) test, p<0.05 as significant. PV1 represents wild-type poliovirus type 1 mahoney strain. Tg21 PVR interferon α/β receptor knockout (IFNAR^{-/-}) mice were infected with wild-type (WT) enteroviruses alone or with co-infected mixed WT+eTIP by intramuscular (I.M.) route (n=10).

FIG. 3F schematically summarizes the results of an eTIP safety test in Tg21PVR mice. The comparison of survival curve is performed by Log-rank (Mantel-Cox) test, p<0.05 as significant. PV1 represents wild-type poliovirus type 1 mahoney strain. Tg21 PVR interferon α/β receptor knockout (IFNAR^{-/-}) mice were infected with wild-type (WT) enteroviruses alone or with co-infected mixed WT+eTIP by intramuscular (I.M.) route (n=10).

FIGS. 4A-4G schematically summarize the results of experiments performed to demonstrate that co-infected PV1 with eTIP increase the survive rate of infected in immune competent animals by I.P. infection.

FIG. 4A is a graph illustrating the protection effect of eTIPs by I.P. inoculated in infected animals. Tg21PVR strain were infected with 10⁷ PFU PV1 alone or with co-infected mixed PV1+eTIP at ratio 1:10 by I.P. route. The number of mice is 17 for Tg21PVR (n=17), Pooled two independent experiments. Co-infected PV1 with eTIP increases the survive rate of infected immune competent animals (Tg21PVR strain). Dash line with solid squares represents co-infected group.

FIG. 4B is a graph illustrating the protection effect of eTIPs by I.P. inoculated in infected animals. IFNAR^{-/-} mice were infected with 10⁴ PFU PV1 alone or with co-infected mixed PV1+eTIP at ratio 1:10. by I.P. route. The number of mice is 7 to 8 for IFNAR^{-/-}mice (n=7-8). Black line with dot represents PV1 alone. Co-infected PV1 with eTIP increases the survive rate of infected immune competent animals (Tg21PVR strain) (FIG. 4A), but not mice lacking the type 1 interferon (IFNAR^{-/-}) receptor. Dash line with solid squares represents co-infected group.

FIG. 4C is a graph illustrating neutralization antibody titer of the infected animals. Sera were collected from the survived mice at 28 days post-infection in FIG. 4A, then neutralization antibody titers were tested by plaque-reduction neutralization test or PRNT.

FIG. 4D is a graph illustrating virus replication in spleen at Day 1 and Day 3. Virus were measured by plaque assay on Hela S3 cells, the data represents as PFU per gram tissue. Patterned bar represents 10⁷ PFU PV1, and solid bar represents coinfected PV1 with eTIP at ratio is 1:10 (n.d. represents under-detection). Two tails multiple-t-tests. The comparison of survival curves is performed by Log-rank (Mantel-Cox) test, *p<0.05 as significant. **p<0.01, ***p<0.001. ns, no significant.

FIG. 4E is a graph illustrating virus replication in brain at Day 1 and Day 3. Virus were measured by plaque assay on Hela S3 cells, the data represents as PFU per gram tissue. Patterned bar represents 10⁷ PFU PV1, and solid bar represents as coinfected PV1 with eTIP at ratio is 1:10 (n.d. represents under-detection). Two tails multiple-t-tests. The comparison of survival curves is performed by Log-rank (Mantel-Cox) test, *p<0.05 as significant. **p<0.01, ***p<0.001. ns, not significant.

FIG. 4F is a graph illustrating virus replication in kidney at Day 1 and Day 3. Virus were measured by plaque assay on Hela S3 cells, the data represents as PFU per gram tissue. Patterned bar represents as 10⁷ PFU PV1, and solid bar represents coinfected PV1 with eTIP at ratio is 1:10 (n.d. represents under-detection). Two tails multiple-t-tests. The comparison of survival curves is performed by Log-rank (Mantel-Cox) test, *p<0.05 as significant. **p<0.01, ***p<0.001. ns, not significant.

FIG. 4G is a graph illustrating virus replication in liver at Day 1 and Day 3. Virus were measured by plaque assay on Hela S3 cells, the data represents as PFU per gram tissue. Patterned bar represents 10⁷ PFU PV1, and solid bar represents coinfected PV1 with eTIP at ratio is 1:10 (n.d. represents under-detection). Two tails multiple-t-tests. The comparison of survival curves is performed by Log-rank (Mantel-Cox) test, *p<0.05 as significant. **p<0.01, ***p<0.001. ns, not significant.

FIG. 5A is a schematic illustrating primary embryo fibroblast cells (MEFs) infected with eTIP alone or with the co-infected eTIP with PV1.

FIG. 5B is a graph showing virus titers measured by plaque assay at M.O.I=0.1, or with ratio at PV1+eTIP=1:10, PV1+eTIP=1:100. At 48-hours post-infection, eTIP inhibit PV1 replication around two-folds. The eTIP titer on Hela S3 cells as measured by GFP positive cells at 9 hours post-infection. Y-axis represent as IU/ml.

FIG. 5C schematically summarizes the results of experiments performed to illustrate that co-infected with eTIP and PV1 significantly increases the interferon induction with the ratio 1:10 (p<0.05, n=3) at post 48-hour infection. Two tails student-t-test, P<0.05 represents as significant.

FIGS. 6A-6E schematically summarize the results of experiments performed to illustrate the protection effect of eTIP by intranasally inoculation in infected animals.

FIG. 6A is a graph illustrating the protection effect of eTIP in infected animals. Immune competent mice (Tg21PVR strain) were infected with 2×10⁵ PFU PV1 alone or with co-infected mixed PV1+eTIP at ratio 1:30 by intranasal (I.N.) route. The number of mice is eleven to seventeen (n=11-17). Dash line with solid squares represents co-infected group. UVed-treated eTIP: UVed for 2 hours.

FIG. 6B is a graph illustrating the effect of eTIP in infected animals. Immune compromise mice (IFNAR^{-/-}) were infected with 10⁴ PFU PV1 alone or with co-infected mixed PV1+eTIP at ratio 1:30 by intranasal (I.N.) route. UVed-treated eTIP: UVed for 2 hours. The number of mice is seven to eight (n=7-8). Solid line with dot represents PV1 alone. Dash line with solid square represents co-infected group.

FIG. 6C is a graph illustrating prophylactic effect of eTIP. 6×10⁶ IU DIP was inoculated into Tg21PVR mice by intranasal (I.N.) route. At 48 hours after inoculation, mice were infected with 2×10⁵ PFU PV1 by Intranasal (I.N.) route. Solid line with dot represents PV1 alone. Dash line with solid squares represents 48h-pretreat.

FIG. 6D is a graph illustrating therapeutic effect of eTIP. Tg21PVR mice were infected with 2×10⁵ PFU PV1 by intranasal (I.N.) route, and then 6×10⁶ IU eTIP were inoculate by Intranasal (I.N.) route from one to five days post-infection (one time daily). The number of mice is sixteen to twenty-one (n=16-21).

FIG. 6E is a graph illustrating that eTIP protects on flu infection (n=7-8). Tg21PVR were inoculated with 10⁵ PFU H1N1 influenza A virus intranasally, PR8 strain alone (PR8), coinfected PR8 with eTIP at ratio is 1:100. Each mouse was weighed daily and normalized to the initial body weight. The comparison of survival curves is performed by Log-rank (Mantel-Cox) test, *p<0.05 as significant. **p<0.01, ***p<0.001. ns, no significant. Pooled two independent experiments in FIGS. 6A, 6C, and 6D.

FIG. 7A schematically summarizes the results of experiments performed to illustrate that, during replication, RNA virus produces defective viral genomes (DVG) that can attenuate parental virus replication and pathogenesis.

FIG. 7B is a schematic representation of the wildtype poliovirus (PV1) and the engineered DVG genome, herein called eTIP1. The structural genes (capsid) encode viral capsid proteins, and the non-structural coding region encodes the enzymatic machinery required for replication. eTIP1 carries a large deletion of ˜1,700 bases in the capsid proteins of PV1 virus, and GFP-Venus gene was inserted at the N-terminus of the engineered viral polyprotein.

FIG. 7C is a schematic illustrating production of eTIP1 particles. In vitro transcribed RNA was transfected into a packaging cell line that expresses the precursor for poliovirus capsid proteins (HelaS3/P1). eTIP1 particles were passaged three times in HelaS3/P1 cells to generate higher titer eTIP1 stocks (˜10⁷ infectious units/ml).

FIG. 7D shows immunohistochemistry analysis of HeLa cells infected with eTIP1 at an M.O.I.=1. At 24 hr post-infection, HeLa S3 cells were fixed and analyzed by immunostaining with antibodies to polio-3A antibody (red), GFP (green), and DAPI (blue).

FIG. 7E is a graph showing that eTIP1 inhibits a wide range of enterovirus sub-species in cell culture (e.g., PV1 and PV3, coxsackievirus B3 (CVB3), enterovirus A71 (EV-A71), enterovirus D68 (EV-D86), rhinovirus 16 (HRV16), rhinovirus 1A (HRV1A), and SARS-CoV-2.

FIG. 8A is a graph showing eTIP1 protects against poliovirus (PV1) in immune-competent wildtype mice. Intraperitoneal inoculation in immune-competent Tg21PVR mice with 10⁷ PFU poliovirus (PV1) or co-infected eTIP1 at a ratio of 1:10. As a control, PV1 was co-inoculated UV-inactivated eTIP1 (UV/eTIP1). The statistical analysis of survival curves was performed by log-rank (Mantel-Cox) test. ns, not significant.

FIG. 8B is a graph showing eTIP1 protects against poliovirus (PV1) in immune-competent wildtype mice. Tg21PVR strain was infected with 3×10⁵ PFU PV1 by the intranasally (IN) or co-infected eTIP1 or UV/eTIP1, at ratio 1:20. The statistical analysis of survival curves was performed by log-rank (Mantel-Cox) test. ns, not significant.

FIG. 8C is a graph showing eTIP1 protects against poliovirus (PV1) in immune-competent wildtype mice. Tissue distribution of the co-infected PV1 with eTIP1 intranasally (IN) at ratio of 1:20. Tissues were collected at indicated times, homogenized and tittered by plaque assay. n=3-5. Data in FIG. 8C were analyzed using unpaired student t tests. n.d. (not detected).

FIG. 8D is a graph showing prophylactic and therapeutic effects of eTIP1. For prophylactic effect, 6×10⁶ IU eTIP1 was inoculated into Tg21PVR mice by IN route, and at 48 h post-infection, mice were infected with 3×10⁵ PFU PV1 by IN route. For therapeutic effect, Tg21PVR mice were infected with 3×10⁵ PFU PV1 by IN route, and then 6×10⁶ IU eTIP1 were inoculated by IN route 1-5 days post-infection, one time daily. n=16-21. Two independent experiments. The statistical analysis of survival curves was performed by log-rank (Mantel-Cox) test.

FIG. 9A is a mRNASeq transcriptome profiling of the lung and spleen from immune competent Tg21PVR mice inoculated with eTIP1 particles. Tg21PVR mice were infected with 6×10⁶ IU eTIP1 particles or PBS (mock) for 48 h. mRNA from lung and spleen were isolated and analyzed by RNASeq and represented as a volcano plot of the genes with significant changed in expression levels, compared to the mock-treated group. Heatmap of the interferon-induced genes for the genes is shown as fold-changes, compared to the mock-treated group (FDR, q-value<0.05).

FIG. 9B is a graph showing flow cytometry for quantification of the immune cells in lung with eTIP1 particles or mock intranasal inoculation. Data are presented as fold-changes, eTIP1 inoculation over mock. n=3-4. Data are from two independent experiments. Unpaired student t tests. n.s., not significant.

FIG. 9C is a graph showing eTIP1 does not protect against poliovirus (PV1) intraperitoneally (IP) in mice lacking a type I interferon response (IFNAR^{-/-}). IFNAR^{-/-} mice were infected with 10⁴ PFU PV1 alone or co-infected with mixed PV1+eTIP1 at a ratio of 1:10 by IP or at a ratio of 1:20 by IN route. n=7-10. Pooled results of two independent experiments. The comparison of survival curves was performed by log-rank (Mantel-Cox) test. ns, not significant.

FIG. 9D is a graph showing eTIP1 does not protect against poliovirus (PV1) intranasally (IN) in mice lacking a type I interferon response (IFNAR^{-/-}). IFNAR^{-/-} mice were infected with 10⁴ PFU PV1 alone or co-infected with mixed PV1+eTIP1 at a ratio of 1:10 by IP or at a ratio of 1:20 by IN route. n=7-10. Pooled results of two independent experiments. The comparison of survival curves was performed by log-rank (Mantel-Cox) test. ns, not significant.

FIG. 9E is a schematic showing self-replicating eTIP1 RNAs form cytosolic dsRNA intermediates and activate pattern recognition receptors, leading to the production of IFN-stimulated genes. This could also promote a protective antiviral state within the local tissue. The plasma membrane of the infected cells lose integrity and release damage-associated molecular patterns that recruit various types of circulating leukocytes to the site of eTIP1 replication.

FIG. 10A is a schematic representation of the eTIP1 RNA Lipoplex and induction of innate immune responses in the mucosal surfaces of the nasal cavity, and mRNASeq transcriptome profiling of lung and brain from mice inoculated with the eTIP1 RNA. K18-hACE2 mice were inoculated intranasally with 30 μg eTIP1 RNA or mock (PBS with empty Lipoplex). Lung and brain were collected 20 h post-inoculation, and mRNA was isolated from these tissues. Heatmap of the interferon-induced genes after intranasal inoculation of eTIP1 RNA/Lipoplex shown as fold-changes compared to the empty Lipoplex control-treated group (FDR, q-value<0.05), n=3, Rep-1 to 3 represent as 3 replicates mice.

FIG. 10B shows immunohistochemistry analysis of mice inoculated intranasally with 30 μg eTIP1 RNA or mock (PBS with empty Lipoplex) or infected with 3×10⁶ infectious units of eTIP1. Heads of inoculated animals were analyzed 24 h post-inoculation by immunohistochemistry. Heads and lungs were collected and fixed in 4% PFA, embedded in paraffin-wax, and cut into 5-μm slides. eTIP1 particles and eTIP1 RNA were stained using poliovirus antibody VPg (3B protein). Poliovirus VPg(3B) (red), ACTUB (green), nuclear (blue). eTIP1 replication was restricted to the upper respiratory nasal cavity. No replication in the lungs was observed.

FIG. 10C is a schematic showing working model of how eTIP1 induces non-autonomous activation of antiviral immunity.

FIG. 10D is graphs showing that eTIP1 inhibits wildtype virus spread into central nervous system (CNS) of infected animals but not replicates in spleen and spinal cord. 6 to 8-weeks-old Tg21 PVR interferon α/β receptor knockout (IFNAR^{-/-}) mice were infected with 200 PFU wildtype poliovirus or co-infected with mixed PV1+eTIP1 at ratio 1:5000 by intramuscular (I.M.) route. RNA genome copies for eTIP1 and PV1 were measured by digital droplet RT-qPCR. Y-axis represents RNA genome copies per 1 mg total RNA. The number of mice is three for each group and each time point (n=3). Two tails multiple-t-tests, P<0.05 represents as significant.

FIG. 11A is graphs showing that eTTP RNAs inhibit SAR-CoV-2 replication in infected mice. 30 μg of eTIP1 RNA or mock (empty Lipoplex) were delivered into K18-hACE2 mice intranasally, and 20 h later, K18-hACE2 mice were challenged with 6×10⁴ PFU SARS-CoV-2, intranasally. Tissues (lung and brain) were collected at 3 days post-infection and homogenized, and supernatants were tittered by plaque assay in Vero-E6 cells. Total RNA was extracted and quantified by qRT-PCR with primers target to Nucleocapsid (N) gene of SARS-CoV-2, normalized to GAPDH. n=6. Unpaired student t tests.

FIG. 11B is immunohistochemistry staining for SARS-CoV-2 in lung of infected animals. Lung and brain tissues were collected at days 3 and 6 post-infection, fixed in 4% PFA, embedded in paraffin-wax, and cut into 5-μm slices. Slides were stained with antibodies that recognized SARS-CoV-2 Nucleocapsid Protein (NP, red) and Spike proteins (SP, grey). ACTUB (green), nuclear (DAPI, blue). Expression levels of SP, NP proteins were qualified by Fuji/Image J with mean intensity and normalized to the SARS-CoV-2 infected group. For each image, at least 10 areas at same places in the different groups were selected. Unpaired student t tests, p-values for each comparison.

FIG. 11C is immunohistochemistry staining for SARS-CoV-2 in brain of infected animals. Lung and brain tissues were collected at days 3 and 6 post-infection, fixed in 4% PFA, embedded in paraffin-wax, and cut into 5-μm slices. Slides were stained with antibodies that recognized SARS-CoV-2 Nucleocapsid Protein (NP, red) and Spike proteins (SP, grey). ACTUB (green), nuclear (DAPI, blue). Expression levels of SP, NP proteins were qualified by Fuji/Image J with mean intensity and normalized to the SARS-CoV-2 infected group. For each image, at least 10 areas at same places in the different groups were selected. Unpaired student t tests, p-values for each comparison.

FIG. 11D is a graph showing relative intensity of the immunohistochemistry staining in FIG. 11B.

FIG. 11E is a graph showing relative intensity of the immunohistochemistry staining in FIG. 11C.

FIG. 12A is a graph showing eTIP reduces the symptoms and lung damage of COVID-19 disease in infected mice. 30 μg of eTIP1 RNA or mock (empty Lipoplex) were delivered into K18-hACE2 mice intranasally, and 20 h later, K18-hACE2 mice were challenged with 6×10⁴ PFU SARS-CoV-2, intranasally. Weight changes were normalized to the initial weight for each mouse. n=9 for each condition in two independent experiments. After animals lost 15% of their body weight, they were humanly euthanized.

FIG. 12B is immunohistochemistry staining of mice in FIG. 12A. Lung tissues were collected at days 3 post-infection, fixed in 4% PFA, embedded in paraffin-wax, and cut into 5-μm slices. Slides were hematoxylin and eosin (H&E) stained sections of lung from K18 hACE2 mice.

FIG. 12C is a graph evaluating tissue sections for comprehensive histological changes and inflammation progression.

FIG. 13A is a graph showing an adjuvant property of eTIP. Intranasal delivery of eTIP enhances production of anti-SARS-CoV-2 neutralizing antibodies.

FIG. 13B is a graph showing an adjuvant property of eTIP. Intranasal delivery of eTIP enhances production of anti-SARS-CoV-2 neutralizing antibodies. eTIP enhances immunogenicity elicit by inactivated and purified SARS CoV2 vaccine.

FIG. 14A shows that eTIP1 protects against poliovirus (PV1) in lung cell types Calu-3 and A549-Ace2 cells with pretreatment. 10⁵ Calu-3 cells in 24 wells were treated with eTIP1 particles at MOI=5, 5 hours later, cells were washed twice with PBS, then cells were infected with poliovirus or SARS-CoV-2 at moi=0.1. Supernatant were collected at indication time-points, virus replication were measured by plaque assay on HelaS3 for PV1, or on Vero-E6 for SARS-CoV-2. Log-value were compared with multi-student t tests. *p<0.05 as significant. **p<0.01, ** *p<0.001s, no significant. n=3.

FIG. 14B shows that eTIP1 protects against SARS-CoV-2 in lung cell types Calu-3 and A549-Ace2 cells with pretreatment. 10⁵ Calu-3 cells in 24 wells were treated with eTIP1 particles at moi=5, 5 hours later, cells were washed twice with PBS, then cells were infected with poliovirus or SARS-CoV-2 at moi=0.1. Supernatant were collected at indication time-points, virus replication were measured by plaque assay on HelaS3 for PV1, or on Vero-E6 for SARS-CoV-2. Log-value were compared with multi-student t tests. *p<0.05 as significant. **p<0.01, ***p<0.001. ns, no significant. n=3.

FIG. 15 is a schematic showing eTIP1 RNA transfection and expression in cell. eTIP1 RNA expression in cell culture model. 2 g eTIPI RNAs were transfected into HelaS3 cells with lipofectamine 2000, then immunofluorescence (IF) staining with the poliovirus-3A antibody at 8 hours post-transfection. Poliovirus 3A protein staining (Red), eTIP1 (green), the nuclear (blue).

FIG. 16 is graphs showing eTIPI RNAs induces interferon induced genes Mx1 and IFN stimulated gene 56 (ISG56) in tissues. K18-hACE2 mice were transfected with 30 g eTIP1 RNA or mock (PBS with empty lipofectamine 2000) by lipofectamine 2000 for 20 hours. Lung and brain were collected, total RNAs were extracted with Trizol reagents. qRT-PCR were performed to qualify the interferon induced genes Mx1 and IFN induced gene 56, ISG56(IFIT1), n=3, normalized to GAPDH. Unpaired student t tests. *p<0.05 as significant. **p<0.01, ***p<0.001. ns, no significant.

FIG. 17 is a graph showing eTIP1 protects influenza H1N1 infection by co-infection intranasally. eTIP1 protects on flu infection (n=7-8). Tg21PVR were inoculated with 10⁵ PFU H1N1 influenza A virus intranasally, PR8 strain alone (PR8), coinfected PR8 with eTIP at ratio is 1:100. Each mouse was weight daily and normalized to the initial body weight. The comparison of survival curves is performed by Log-rank (Mantel-Cox) test, *p<0.05 as significant. **p<0.01, ***p<0.001. ns, no significant.

### DETAILED DESCRIPTION OF THE DISCLOSURE

The present disclosure relates generally to the development of novel antiviral therapies for the treatment of health conditions associates with immune diseases and viral infection. In particular, some aspects and embodiments of the disclosure relate to the development of defective interfering (DI) particles (DIPs) of enteroviruses for use in the prevention and management of various health conditions such as immune diseases and microbial infection. In particular, as described in greater detail herein, the absence of at least a portion of the nucleic acid sequence encoding viral structural proteins was found necessary and sufficient for the DI phenotype. In addition, as illustrated in the Examples, the virus-virus interactions between the populations of wild-type viral particles and DI particles could provide a way of quantitative modulation of immune targets in virus-host interactions in pathogenesis and persistence of viral infection.

Recent outbreaks of various enteroviruses such as D68(EV-D68), flu, and COVID-19 have raised health concern in US and worldwide. Despite many recent advances in developing vaccines for poliovirus, effectively controlling the spread of other infectious diseases, whether bacterial, viral, or parasitic, has been reported very challenging. For example, as the poliovirus eradication campaign, if one stop vaccination of people, one will also need to develop alternative antivirals. On the other hand, the outbreaks of the enteroviruses such as those indicated above illustrates an urgent need to develop new antivirals and vaccines.

A possible approach to prevent virus spreading and cure infectious diseases is the use of viruses themselves. This approach concerns essentially the development of DI particles that can suppress the spread of their intact, replication competent counterparts, reducing infectious virus yields. DIPs were originally identified in the early 50's in various studies of undiluted serial passages of influenza A virus. The inhibition of wild-type virus of the DIP in cell culture level has been observed on the early 80's, but whether the DIPs can used as a therapeutic antiviral and what the mechanisms are still unclear.

DIPs can be generated naturally during virus replication DIPs, and are generally characterized by deletions and/or insertions of nucleotides in the viral genome, which in turns prevents the production of one or more proteins essential for viral spread. DIPs can replicate its RNA genome normally as it maintains the essential part for viral RNA replication, however, it cannot generate infectious progeny because it lacks various portions of the viral genome. For replication, DIPs rely on the presence of homologous helper wild-type virus, sometimes referred to as standard virus (STV), that supplies the missing viral protein(s) in trans. Remarkably, the presence of DIPs has been demonstrated for almost every virus studied, making DIPs a viable option to prevent and/or treat a larger number of viral diseases.

The DIP approach offers several potential benefits over conventional pharmaceutical based therapies and vaccines. Firstly, defective interfering (eTIP) RNAs originate from a viral genome and act by competing with viral genomes for replication or packaging. Their capacity to be packaged provides specific and efficient targeting viruses. Secondly, DIPs replicate faster than wild-type virus due to the smaller/shorter genome in the infected cells. As a result, DIP often outcompete wild-type virus for intracellular viral resources generated by the wild-type virus, which in turns would enable eTIP to transmit more efficiently than wild-type virus from the infected cells or animals. Therefore, it is important to test the effective and safety of DIP in infected animals and to understand its mechanisms for therapeutic antiviral use. Thirdly, host innate immunity is considered important for the first line of defense against viral infection. For example, the human innate immune response, particularly the type-I interferon (IFN) response, is highly robust and effective first line of defense against virus invasion. It still remains to determine whether the protection effect of DIP is due to direct interference with the standard virus (STV) replication or through strong immunostimulatory effect, or the combination of both. As described in greater detail below, a number of eTIP of poliovirus have been designed and evaluated. In particular, as illustrated in Examples 12-13, eTIP can be used as a broad antiviral against a wide ranges of RNA viruses, including poliovirus, non-polio enterovirus, rhinovirus and influenza virus. In particular, the data presented herein demonstrate that eTIP protects the lethal poliovirus at the mucosal surface by intranasal infection as prophylactic administration and/or therapeutic administration. Furthermore, the data presented herein demonstrate that eTIP-induced mucosal immunity in respiratory tract contributes to the protection effect.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The terms “administration” and “administering”, as used herein, refer to the delivery of a bioactive composition or formulation by an administration route comprising, but not limited to, intranasal, transdermal, intravenous, intra-arterial, intramuscular, intraperitoneal, subcutaneous, intramuscular, oral, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a composition of the disclosure, e.g., DIP, nucleic acid construct, or pharmaceutical composition, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about” which, as used herein, has its ordinary meaning of approximately. The term “about” is used to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value.

The term “construct” refers to a recombinant molecule including one or more isolated nucleic acid sequences from heterologous sources. For example, nucleic acid constructs can be chimeric nucleic acid molecules in which two or more nucleic acid sequences of different origin are assembled into a single nucleic acid molecule. Thus, representative nucleic acid constructs include any constructs that contain (1) nucleic acid sequences, including regulatory and coding sequences that are not found adjoined to one another in nature (e.g., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative nucleic acid constructs can include any recombinant nucleic acid molecules, linear or circular, single stranded or double stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. Constructs of the present disclosure can include the necessary elements to direct expression of a nucleic acid sequence of interest that is also contained in the construct. Such elements may include control elements such as a promoter that is operably linked to (so as to direct transcription of) the nucleic acid sequence of interest, and optionally includes a polyadenylation sequence. In some embodiments of the disclosure, the nucleic acid construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in a cell. For the practice of the present disclosure, compositions and methods for preparing and using constructs and cells are known to one skilled in the art.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules described herein or the coding sequences and promoter sequences in a nucleic acid molecule means that the coding sequences and promoter sequences are in-frame and in proper spatial and distance away to permit the effects of the respective binding by transcription factors or RNA polymerase on transcription. It should be understood that, operably linked elements may be contiguous or non-contiguous (e.g., linked to one another through a linker). In the context of polypeptide constructs, “operably linked” refers to a physical linkage (e.g., directly or indirectly linked) between amino acid sequences (e.g., different segments, portions, regions, or domains) to provide for a described activity of the constructs. Operably linked segments, portions, regions, and domains of the polypeptides or nucleic acid molecules disclosed herein may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990).

Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub- combination was individually and explicitly disclosed herein.

### Enteroviruses

Enteroviruses belong to a genus of the Picornaviridae family, a large group of small RNA viruses characterized by a single positive-strand genomic RNA. Enteroviruses are named by their transmission-route through the intestine (enteric meaning intestinal), and are associated with a wide range of symptoms, syndromes, and diseases in humans, mammals, as well as in other animals. These include acute benign pericarditis, acute flaccid paralysis, acute hemorrhagic conjunctivitis, aseptic meningitis, various exanthemas, carditis, croup, encephalitis, enanthema, gastrointestinal disease, hepatitis, hand-foot-and-mouth disease, various respiratory diseases, myocarditis, neonatal disease including multi-organ failure, pericarditis, pleurodynia, rash, and undifferentiated fever. In general, the syndromes are not correlated with particular enterovirus serotypes, nor does a serotype specifically correlate with a particular disease, although in certain cases serotypes do correlate with particular diseases.

Enteroviruses are responsible for large numbers of infections. After rhinoviruses, enteroviruses have been reported to be the most common viral infection in humans. Enteroviral infections lead to large numbers of hospitalizations each year for aseptic meningitis, myocarditis, encephalitis, acute hemorrhagic conjunctivitis, nonspecific febrile illnesses, and upper respiratory infections. Enteroviruses are also implicated in acute flaccid paralysis in animal models, as well as in dilated cardiomyopathy. Six serotypes of coxsackie B viruses are implicated in a variety of clinical diseases, such as meningitis, myocarditis and severe neonatal disease. Recently, enterovirus infection has also been linked to chronic fatigue syndrome. Poliovirus, with three known serotypes, PV1, PV2, and PV3, is another enterovirus known to infect humans.

As the family name indicates, enteroviruses are small RNA viruses. All enteroviruses contain a genome of approximately 7,500 bases and are known to have a high mutation rate due to low-fidelity replication and frequent recombination. The viral single stranded RNA comprises a 5′ nontranslated region, which is followed by an open reading frame coding for a polyprotein precursor of Mr 240-250×10³ Da, followed by a 3′ noncoding sequence and a poly (A) tract. The coding region of the virus is conventionally divided into three sections, referred to as P1, P2, and P3. The P1 region encodes the structural (capsid) proteins. The P2 region encodes proteins required for RNA replication and one of the viral proteinases responsible for host cell shut-off of cap-dependent translation. The P3 region encodes the major viral proteinase (3C^{Pro}), the viral RNA-dependent RNA polymerase (3D^{Pol}), and other proteins required for RNA replication. The coding region is preceded by an unusually long 5′ NCR, which directs translation initiation by internal ribosome entry in the absence of cap-dependent functions. The viral genome also contains a short 3′ NCR, which presumably contains cis-acting sequences involved in template recognition by the viral-replication initiation complex.

In the polyprotein, the sequence of gene products begins 1A, 1B, 1C, 1D, and 2A. 1A through 1D are, respectively, the structural proteins VP4, VP2, VP3, and VP1 of the viral capsid; VP1 is followed in the open reading frame by a nonstructural protein 2A. Upon entry into the host-cell, the viral RNA genome is replicated and translated into a single polyprotein, which is subsequently processed by virus-encoded proteases into the structural capsid proteins and the nonstructural proteins, then the viral genomes are encapsidated into structural capsid shell to generate a mature virus particles. Nonstructural proteins are mainly involved in the replication of the virus.

Serologically distinct enteroviruses were originally distributed into five groups within the enteroviruses: coxsackievirus A (CA), coxsackievirus B (CB), echovirus (E), and numbered enteroviruses (EV), as well as poliovirus (PV). Poliovirus, as well as coxsackie and echovirus, is spread through the fecal-oral route. Infection can result in a wide variety of symptoms, including those of: mild respiratory illness (the common cold), hand, foot and mouth disease, acute hemorrhagic conjunctivitis, aseptic meningitis, myocarditis, severe neonatal sepsis-like disease, acute flaccid paralysis, and the related acute flaccid myelitis.

Enteroviruses are now assigned sequential numbers and grouped based on genetic and phenotypic similarity. To date, more than 110 genetically distinct enteroviruses that infect humans and non-human primates have been identified. The enterovirus genus currently includes the following fifteen species: Enterovirus A (formerly Human enterovirus A), Enterovirus B (formerly Human enterovirus B), Enterovirus C (formerly Human enterovirus C), Enterovirus D (formerly Human enterovirus D), Enterovirus E (formerly Bovine enterovirus group A), Enterovirus F (formerly Bovine enterovirus group B), Enterovirus G (formerly Porcine enterovirus B), Enterovirus H (formerly Simian enterovirus A), Enterovirus I, Enterovirus J, Enterovirus K, Enterovirus L, Rhinovirus A (formerly Human rhinovirus A), Rhinovirus B (formerly Human rhinovirus B), and Rhinovirus C (formerly Human rhinovirus C).

Coxsackievirus includes serotypes belonging to Enterovirus A (exemplary serotypes include CVA-2, CVA-3, CVA-4, CVA-5, CVA-6, CVA-7, CVA-8, CVA-10, CVA-12, CVA-14, and CVA-16); Enterovirus B (exemplary serotypes include serotypes CVB-1, CVB-2, CVB-3, CVB-4, CVB-5, CVB-6, and CVA-9); Enterovirus C (exemplary serotypes include serotypes CVA-1, CVA-11, CVA-13, CVA-17, CVA-19, CVA-20, CVA-21, CVA-22, and CVA-24).

Echoviruses include serotypes belonging to Enterovirus B. Exemplary serotypes include E-1, E-2, E-3, E-4, E-5, E-6, E-7, E-9, E-11 through E-21, E-24, E-25, E-26, E-27, E-29, E-30, E-31, E32, and E-33.

Enteroviruses include serotypes belonging to Enterovirus A (exemplary serotypes include EV-A71, EV-A76, EV-A89 through EV-A92, EV-A114, EV-A119, EV-A120, EV-A121, SV19, SV43, SV46, and BabEV-A13); Enterovirus B (exemplary serotypes include EV-B69, EV-B73 through EV-B75, EV-B77 through EV-B88, EV-B93, EV-B97, EV-B98, EV-B100, EV-B101, EV-B106, EV-B10⁷, EV-B 110 through EV-B 113, and SA5); Enterovirus C (exemplary serotypes include serotypes EV-C95, EV-C96, EV-C99, EV-C102, EV-C104, EV-C10⁵, EV-C109, EV-C 113, EV-C 116, EV-C 117, and EV-C 118); Enterovirus D (exemplary serotypes include EV-D68, EV-D70, EV-D94, EV-D 111, and EV-D120); Enterovirus E (exemplary serotypes include EV-E1, EV-E2, EV-E3, EV-E4, and EV-E5); Enterovirus F (exemplary serotypes include EV-F1, EV-F2, EV-F3, EV-F4, EV-F5, EV-F6, and EV-F7); Enterovirus G (exemplary serotypes include EV-G1 through EV-G20); Enterovirus H (exemplary serotypes include EV-H); Enterovirus I (exemplary serotypes include EV-I1 and EV-I2); Enterovirus J (exemplary serotypes include EV-J1, EV-J103, and EV-J108); Enterovirus K (exemplary serotypes include EV-K1 and EV-K2); and Enterovirus L (exemplary serotypes include EV-L1).

Rhinoviruses include serotypes belonging to Rhinovirus A (exemplary serotypes include RV-A1, RV-A1B, RV-A2, RV-A7 through RV-A13, RV-A15, RV-A16, RV-A18 through RV-A25, RV-A28 through RV-A34, RV-A36, RV-A38 through RV-A41, RV-A43, RV-A45 through RV-A47, RV-A49 through RV-A51, RV-A53 through RV-A68, RV-A71, RV-A73 through RV-A78, RV-A80 through RV-A82, RV-A85, RV-A88 through RV-A90, RV-A94, RV-A96, and RV-A100 through RV-A108); Rhinovirus B (exemplary serotypes include RV-B3 through RV-B6, RV-B14, RV-B17, RV-B26, RV-B27, RV-B35, RV-B37, RV-B42, RV-B48, RV-B52, RV-B69, RV-B70, RV-B72, RV-B79, RV-B83, RV-B84, RV-B86, RV-B91 through RV-B93, RV-B97, and RV-B99 through RV-B104); and Rhinovirus C (exemplary serotypes include serotypes RV-C1 through RV-C51, RV-C54, RV-C55, and RV-C56.

Polioviruses include serotypes belonging to Enterovirus C. Exemplary poliovirus serotypes include PV-1, PV-2, and PV-3. These three serotypes of poliovirus, PV-1, PV-2, and PV-3 each have a slightly different capsid protein. Capsid proteins define cellular receptor specificity and virus antigenicity. PV-1 is the most common form encountered in nature; however, all three forms are extremely infectious. Poliovirus can affect the spinal cord and cause poliomyelitis.

### Defective Interfering Particle

Defective interfering (DI) particles (DIPs) are of viral origin and share the similar structural features as their homologous wild-type viruses (sometimes referred to as standard virus, STV), yet they contain a truncated form of the viral genome. As a result of the missing genomic information, DIPs are defective in virus replication and, hence, cannot result in the production of progeny virions, once introduced into a cell. However, upon complementation by coinfection with a fully infectious homologous STV, interference with the normal viral life cycle can be observed, with suppressed STV replication and the release of mainly noninfectious progeny DIPs. This outcome is a result of the growth advantage of the DI genome over the full-length (FL) counterpart, which is manifested by enhanced genomic replication, out-competition for cellular or viral resources, and preferential packaging into virus particles. Furthermore, considering the ability of DIPs to suppress virus replication, a growing interest in the potential application of DIPs as an antiviral agent has been reported. However, the protective effect of DIPs on wild-type virus in infected animals is not well characterized and whether DIP can be used as an effective antiviral and therapeutic agent is still not very clear.

As presented in more detail below, an exemplary DIP of poliovirus, eTIP, has been generated and tested in cell culture and in infected animals. The experimental data described below demonstrate that eTIP of the disclosure inhibits a wide range of RNA viruses, including different strains of wild-type polioviruses, non-polio enteroviruses, and influenza virus. In particular, the experimental data described herein demonstrate that eTIP can be used as prophylactic antiviral and therapeutic antiviral via intranasal inoculation. Furthermore, the experimental data described herein demonstrate that eTIP induces the host interferon responses which in turns plays an important role for protection against viral infection. Taken together, the experimental data described herein demonstrated that DIPs, exemplified by poliovirus DIP, can confers broad-spectrum antiviral against a wide range of RNA viruses, including poliovirus, non-polio enterovirus, rhinovirus and influenza virus. In particular, the data presented herein demonstrate that eTIP protects the lethal poliovirus at the mucosal surface by intranasal infection as prophylactic administration and/or therapeutic administration. Furthermore, the data presented herein demonstrate that eTIP-induced mucosal immunity in respiratory tract contributes to the protection effect.

Provided herein is DVGs that can be harnessed to develop safe broad spectrum antivirals that protect therapeutically and prophylactically against diverse viral infections. In an embodiment, provided herein is eTIP1, a nanoparticle DVG that can be administered intranasally to combat a range of respiratory virus infections, including several enterovirus, COVID-19 and Flu, without detrimental side effects to the host. Because eTIP1 intranasal inoculation can offer protection even if administered 48 hours before infection or 24 hours after challenge, it can provide an effective therapeutic window that compares favorably with small molecule antivirals.

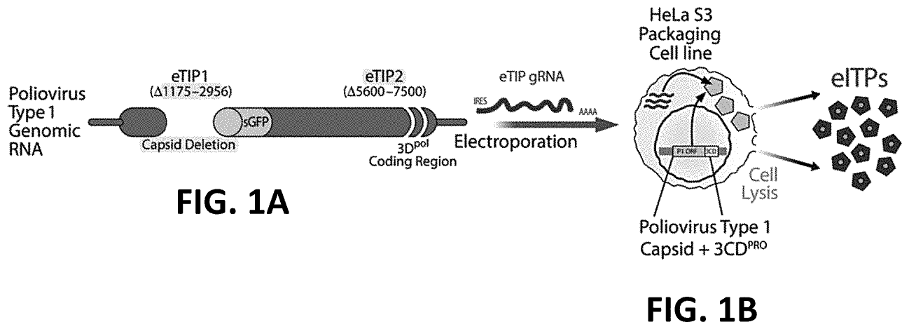

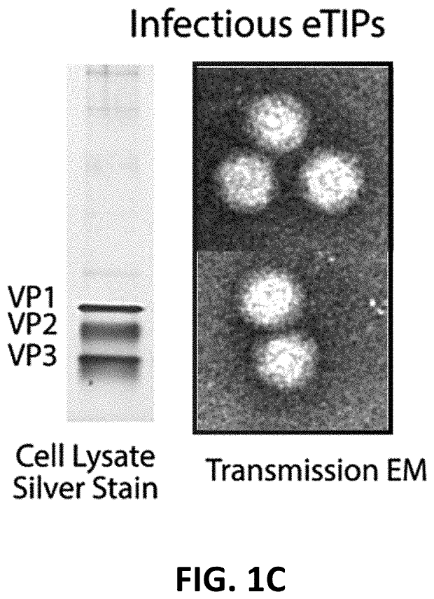

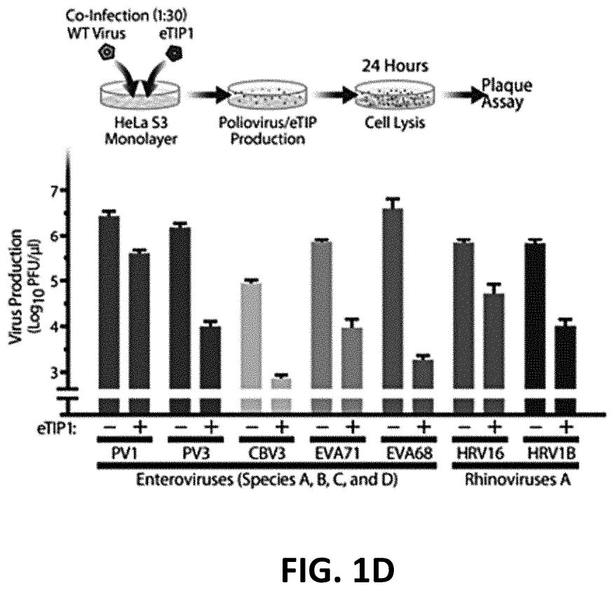

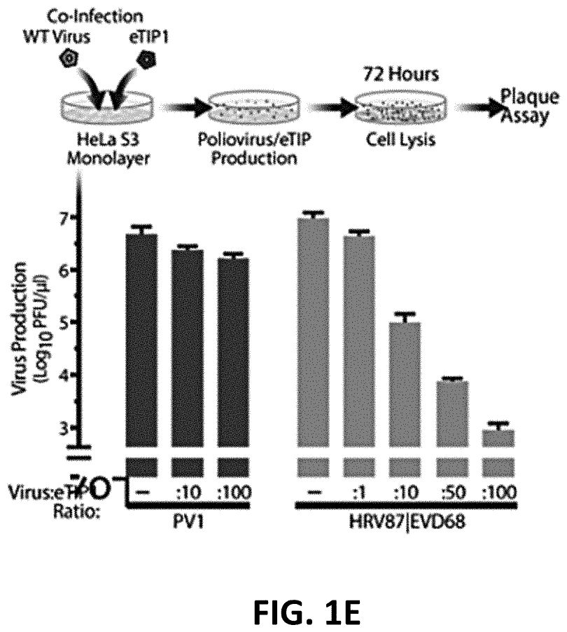

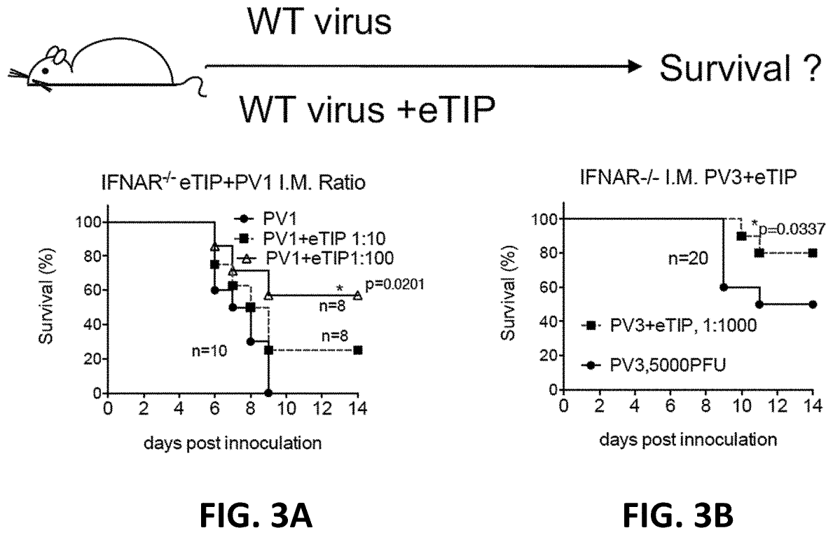

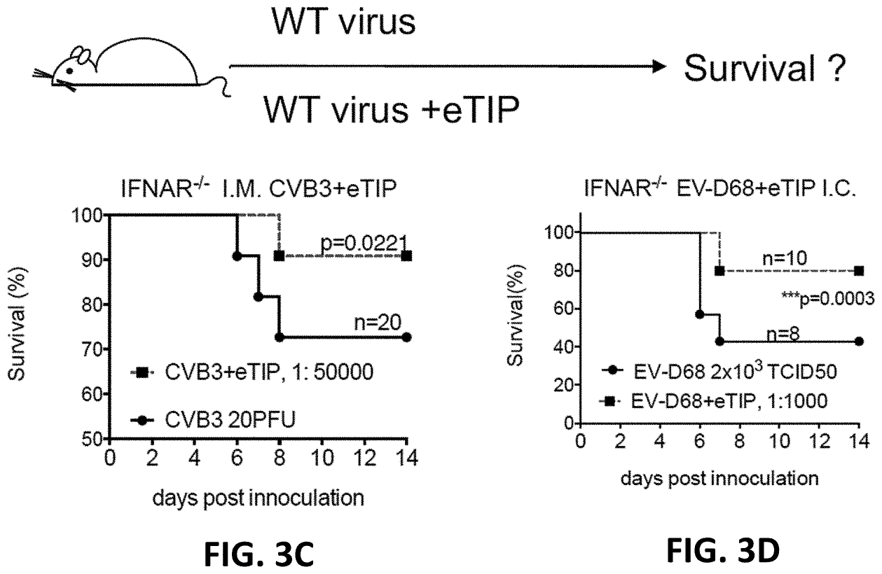

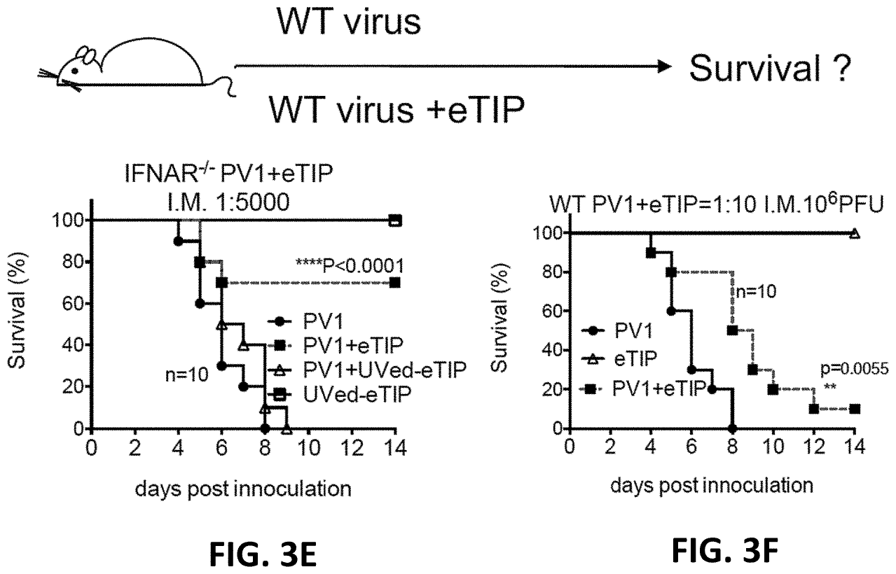

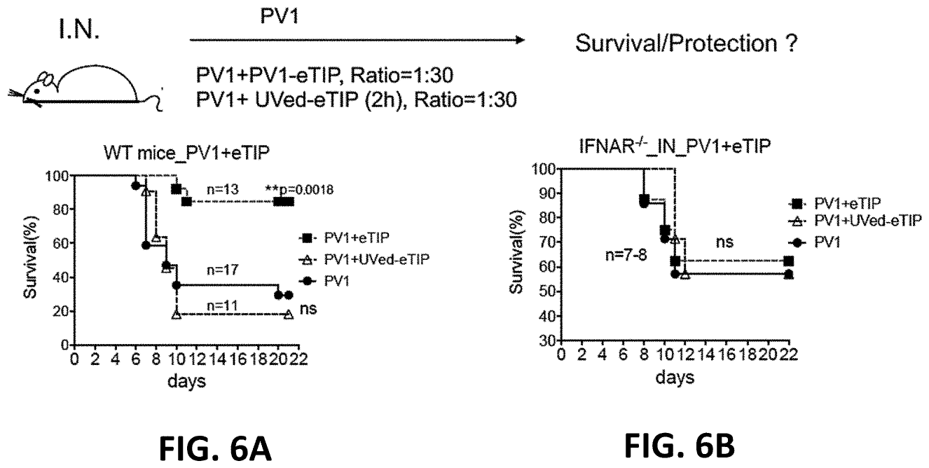

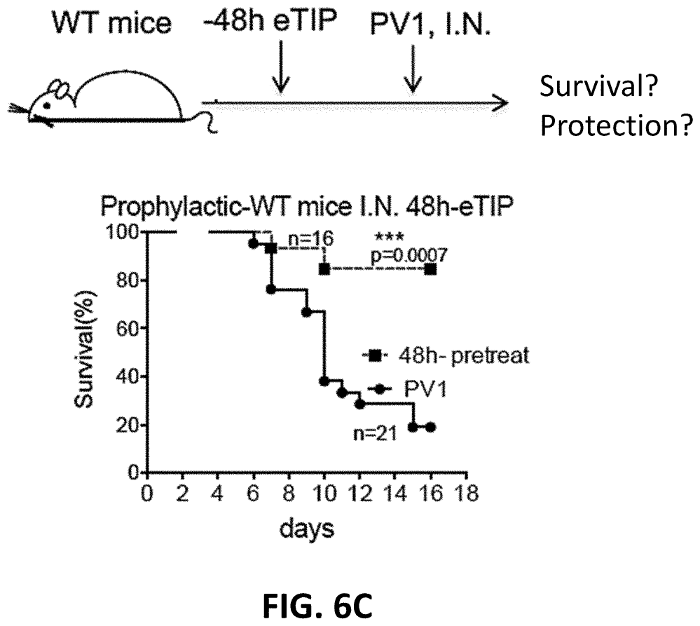

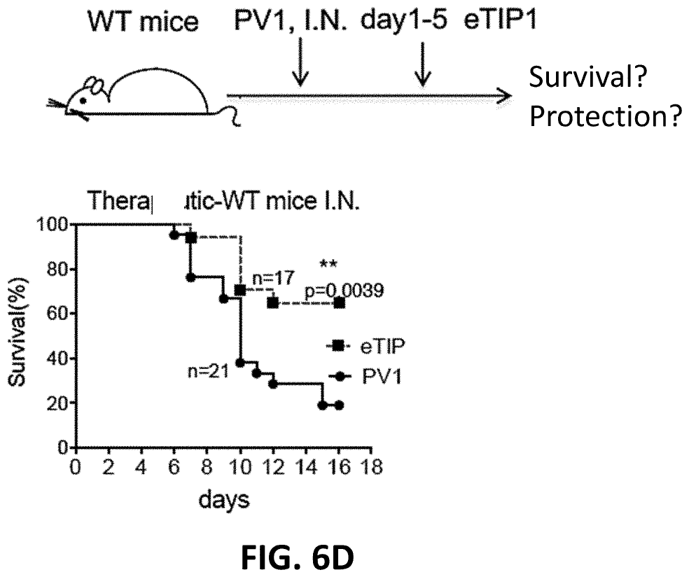

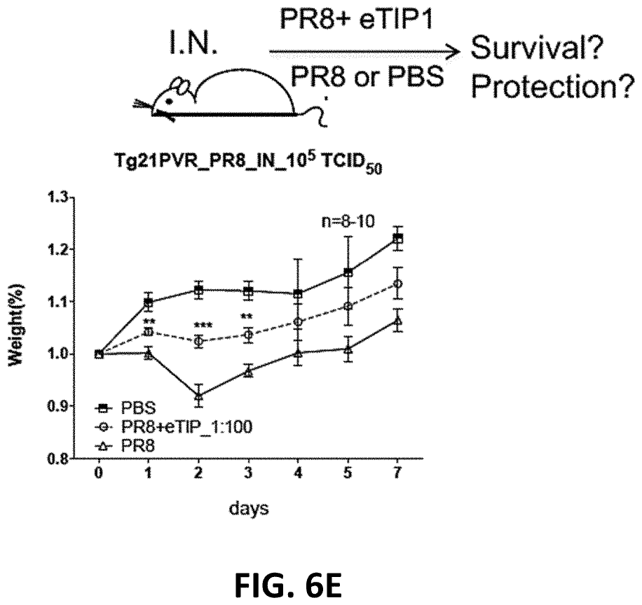

As described in greater detail in the Examples below, certain DIPs of the disclosure are suitable for use as a therapeutic antiviral strategy. In particular, highly effective eTIP have been developed and demonstrated as indicated by being capable of reducing infection by 10 to 100-fold for a wide range of both enterovirus and rhinovirus in cell culture, as tested by coinfection. Experimental data presented herein also demonstrate that the eTIP disclosed herein is safe and protects animals from infection and death caused by different enterovirus (see, e.g., FIGS. 1-3) and rhinovirus (see, e.g., FIGS. 1 and 3) without themselves causing any disease. In the immune-compromised mice, the competition effect appear to correlate with (i) the similarity to the genomic sequences, (ii) the efficiency of the packaging, and/or (iii) the initial ratio that was delivered to animals. Furthermore, the experimental data presented herein demonstrate that the eTIPs can act both therapeutically (see, e.g., FIG. 6) and prophylactically. Specifically, the experimental data presented herein demonstrate a broad-spectrum antiviral therapy: eTIP cross-protects from a number of enteroviruses (e.g., coxsackievirus B3, other poliovirus serotype strains, EV-D68) and H1N1 influenza virus in animal models. In particular, the most robust protection was observed at mucosal surfaces following intranasal inoculation. In addition, it was observed that eTIP can protect mice from intranasal inoculation even if eTIP is administered 48 hours before challenge with wild-type viruses (prophylactic effect) or continuously 5 days after challenge (therapeutic effect).

Mechanistic experiments were also carried out to investigate why and how eTIPs exert their protective effects in infected animals (see, e.g., FIGS. 3, 4, and 5). It was observed that replication of eTIP is required for protection. In overall, this mechanistic understanding allows applicant to improve the DIP design and translate these findings to other virus families. Specifically, the experimental data described herein demonstrated that: (i) the pretreatment with eTIP induces the interferon responses (as shown in host mRNA transcriptome studies); (ii) the interferon responses play an important role in the protection in the wild-type mice; and (iii) the induction of local innate immunity responses play important role on eTIP inhibits virus replication.

Despite the enormous success of antimicrobial pharmaceuticals and vaccines, effectively controlling the spread of infectious diseases, whether bacterial, viral, or parasitic, has proved exceptionally challenging. As the poliovirus eradication campaign, if one stop vaccination of people, one will need to develop alternative vaccine and antivirals. The generation of new or improved vaccines to prevent virus disease in people, domestic animals, and farmed stock remains a considerable challenge, as the outbreak of the emerging virus, vaccines are not always effective, and testing it will time-consuming. Viruses have a disquieting ability to become resistant to the limited range of antivirals currently are available. Although there are controversial discussion of the eTIP as the antiviral of the influenza virus, the eTIP approach offers several potential benefits over conventional pharmaceutical-based therapies and vaccines.

Additional analysis of the interaction between eTIP and wild-type genome are contemplated to gain additional insight into the basic mechanisms of viral replication as well as those of the modulation of the viral infection process in susceptible organisms. Without being bound to any particular theory, it is believed that eTIPs replicate preferentially at the expense of the helper virus by competing with helper virus-encoded replication and structural proteins and can facilitate the establishment and maintenance of persistent virus infections of cells. It is also hypothesized that these eTIP particles may be related to the molecular mechanisms of genetic recombination in RNA genomes. Generation of eTIP genomes could occur by nonhomologous recombination, probably by a copy-choice mechanism.

The third possibility, the most importantly, is that eTIP or the virus-eTIP co-infection may plays a role in regulating innate and adaptive immunity. In particular, as described in greater detail below, in pretreatment experiments of wild-type mice with eTIP by intranasal infection, it was observed that in a 48-hours pretreatment, the protection rate of the eTIP was 80%, whereas in a 24-hours pretreatment, no significant protection effect was observed. The eTIPs can be used as a stimulate of the host interferon responses to enhance the eTIP protection effect on viruses. Interferon responses were also observed in spleen, which suggests that cell-type specific immune responses (e.g., dendritic cells, macrophages, or T cells responses) were induced in the context of these conditions. It is contemplated that eTIP can be used as an antiviral and broad vaccine to stimulate the mucosal immunity and cell type specific immunity. It is contemplated that eTIP can also be used as the vaccine adjuvant.

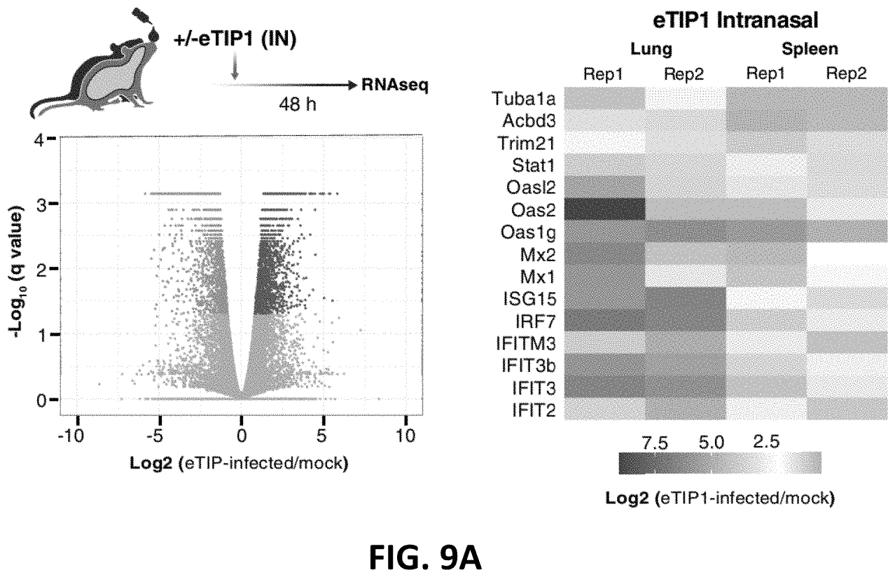

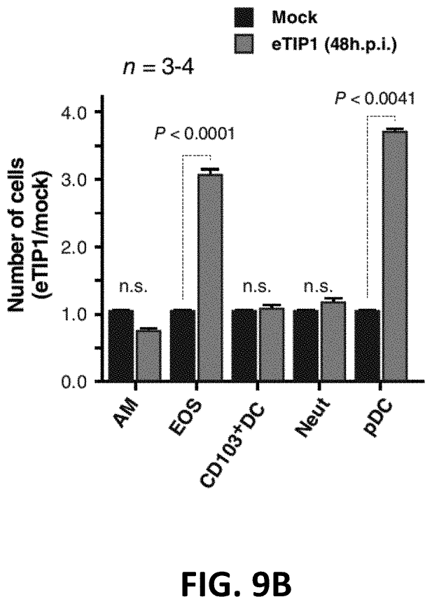

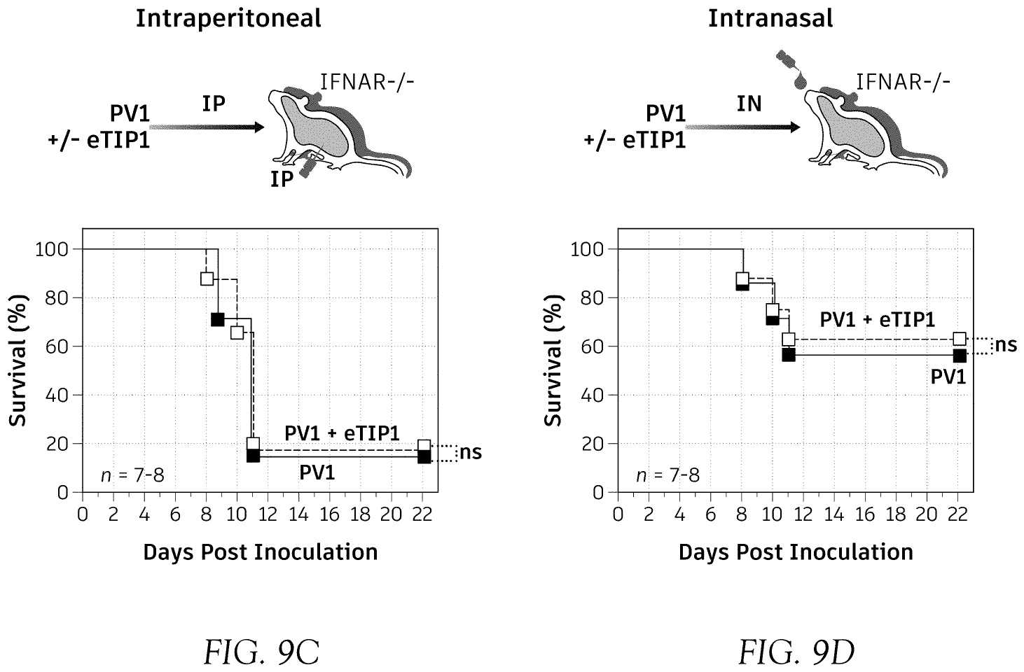

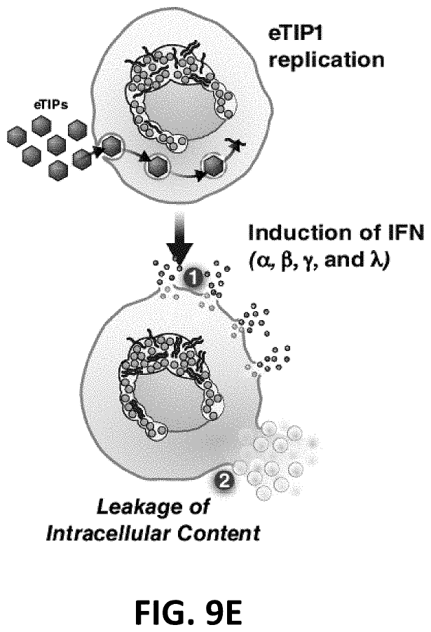

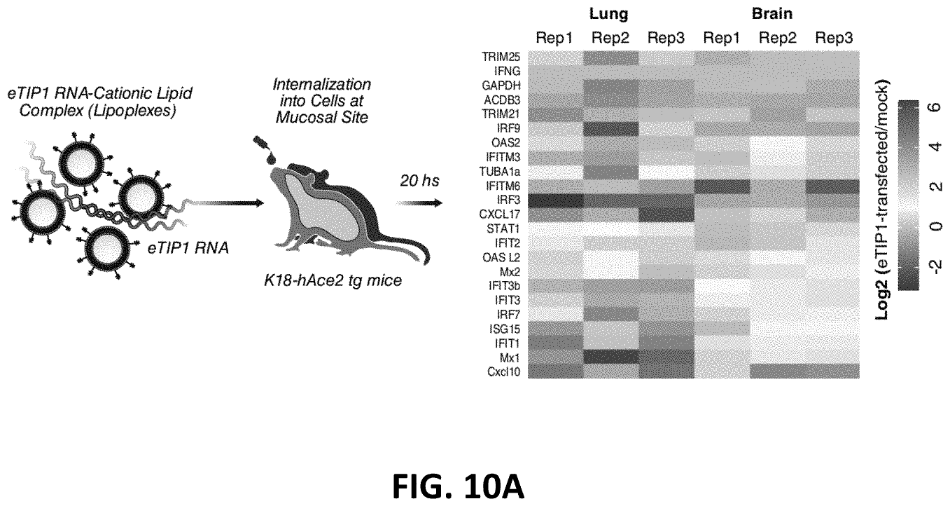

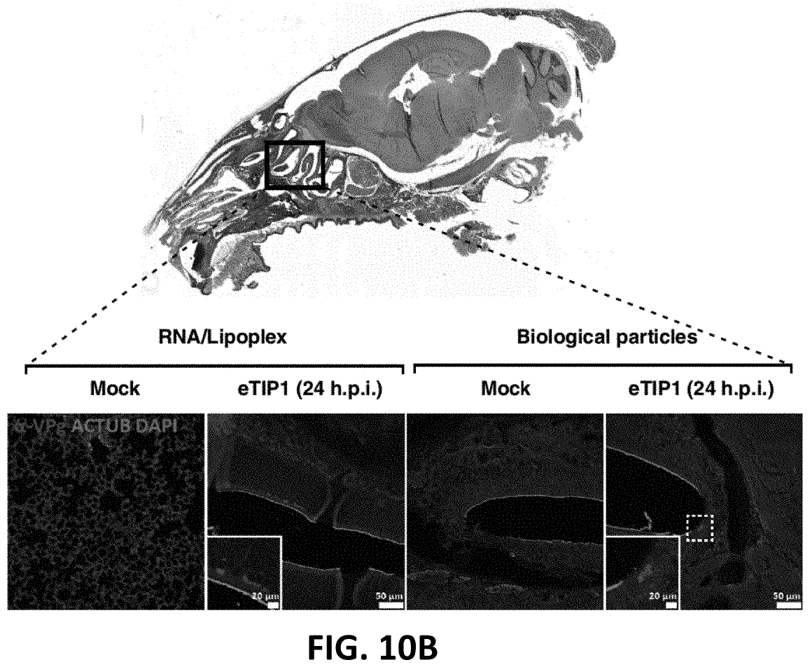

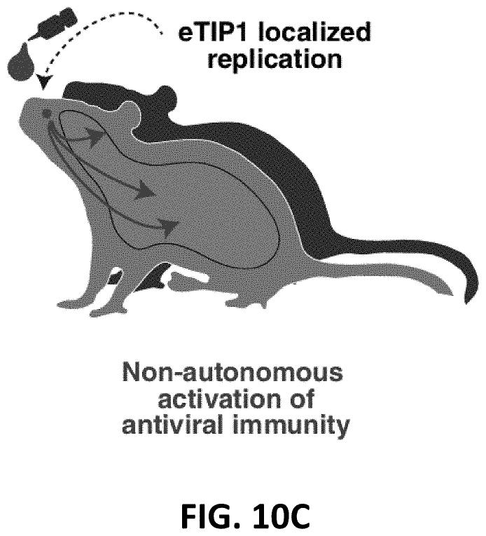

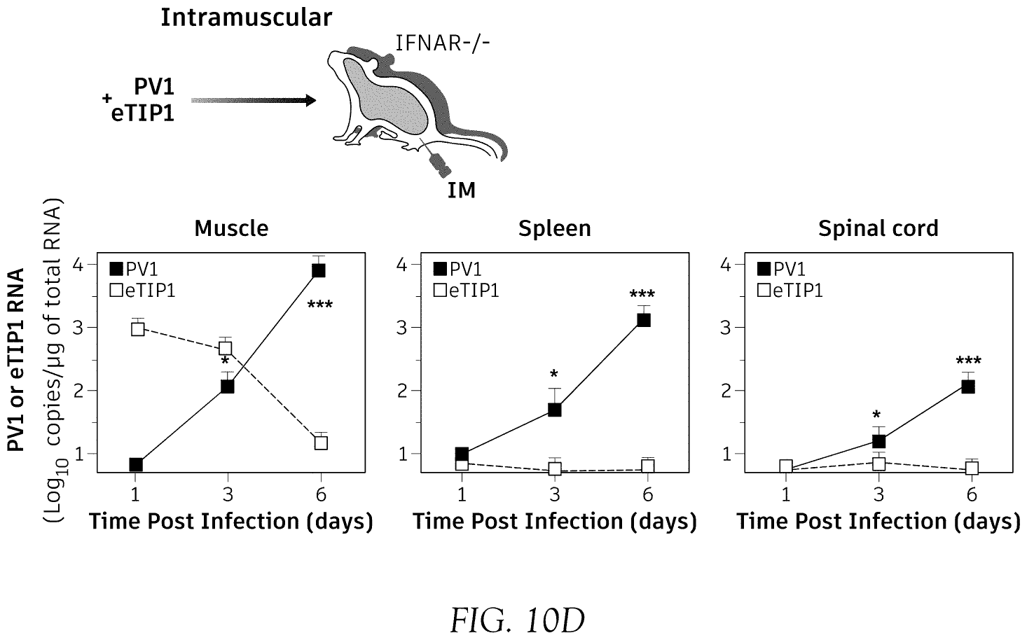

While DVGs were thought to modulate infection by competing with their parental virus for host resources, experiments provided herein indicate that eTIP1 protective mechanism of action depends on induction of innate responses. Consistent with this idea, data provided herein show that eTIP1 treatment does not protect animals defective in type I interferon (FIGS. 9C-9D). Furthermore, eTIP1 offers highly effective protection against viruses from different families in a non-cell autonomous manner (FIGS. 10A-10D), consistent with herein provided proposed mechanism of action.

Viral infection presents several unique challenges to antiviral development, most notably the mutational plasticity that renders most drug treatments and even antibody therapies ineffective. Vaccination, the only available tool to combat directly viral infection, harnesses the natural defenses of the organism. No similar approach is currently available for prophylaxis or treatment. Provided herein is, for example, eTIP1, which provides a new concept in antiviral development that meets this urgent need. eTIP1 can offer several potential benefits over conventional pharmaceutical based therapies, such as small-molecule antivirals or monoclonal antibodies. While viruses have a great ability to become resistant to the limited range of antivirals or antibodies currently available, a single dose of eTIP1 can recruit the host's own antiviral defenses to create an antiviral state that lasts for at least a few days. eTIP1 can induce a multicomponent antiviral response, in the form of diverse ISGs, which should be refractory to the development of resistance through viral mutation.

In some embodiments, provided herein is eTIP, e.g., eTIPI, with a number of important safety features. First, eTIP1 is based on a safe poliovirus backbone that cannot itself cause disease and can be administered non-invasively. Secondly, while eTIPI mimics a benign infection, its replication is limited to few cells near the site of inoculation (FIGS. 10B and 10D)). Therefore, eTIP2 does not cause disease, but elicit a non-cell autonomous protective immune response (FIG. 10C). Thirdly, because eTIP1 can self-amplify in the few cells it enters, it requires a lower amount of eTIP1 RNA to induce full non-cell autonomous antiviral protection. Finally, the nanoparticle formulation of the eTIP1 circumvents any concerns that pre-existing immunity will prevent repeated courses of administration.

Another key feature of eTIP1 protective action is that it can induce a balanced and non-detrimental antiviral state. Indeed, there was no weight loss or signs of distress in any of the animals treated with eTIPI, and even those co-infected with SARS showed no sign of disease. In contrast, antiviral treatments relying on administration of interferons or dsRNA mimetic molecules, such as poly(I:C) or 5′triphosphate dsRNA are reported to have serious side effects such as fever, headache, fatigue, arthralgias, and myalgias. Therefore, eTIP1 provided herein can achieve a balanced regulation of innate responses, which is important to avoid the detrimental effects of interferon administration.

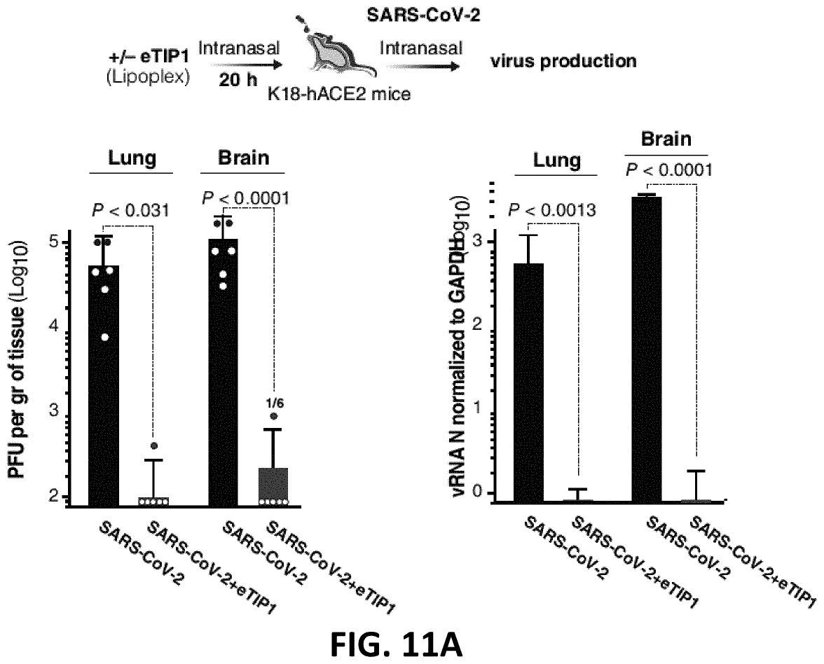

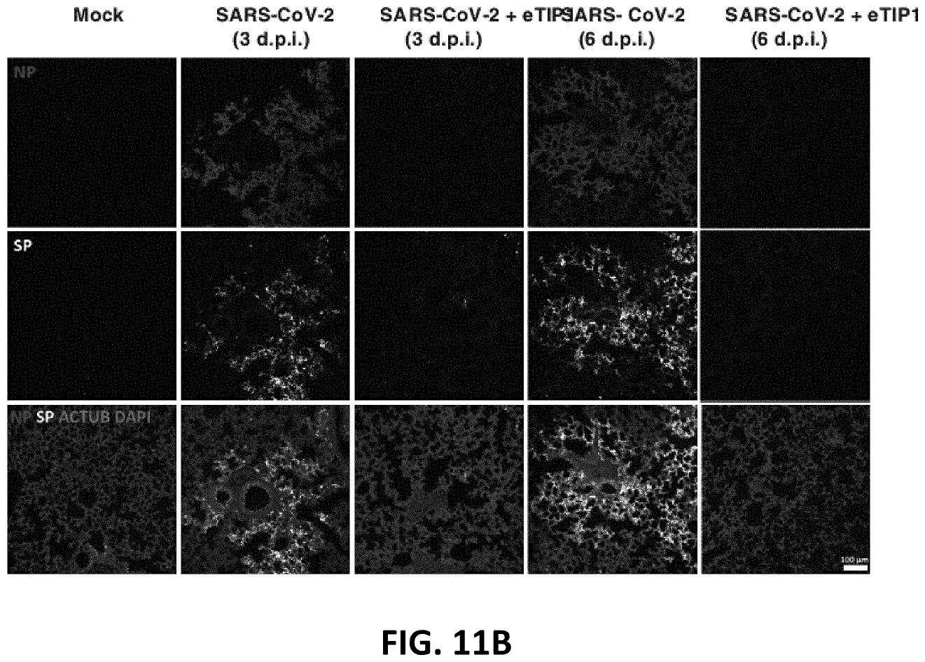

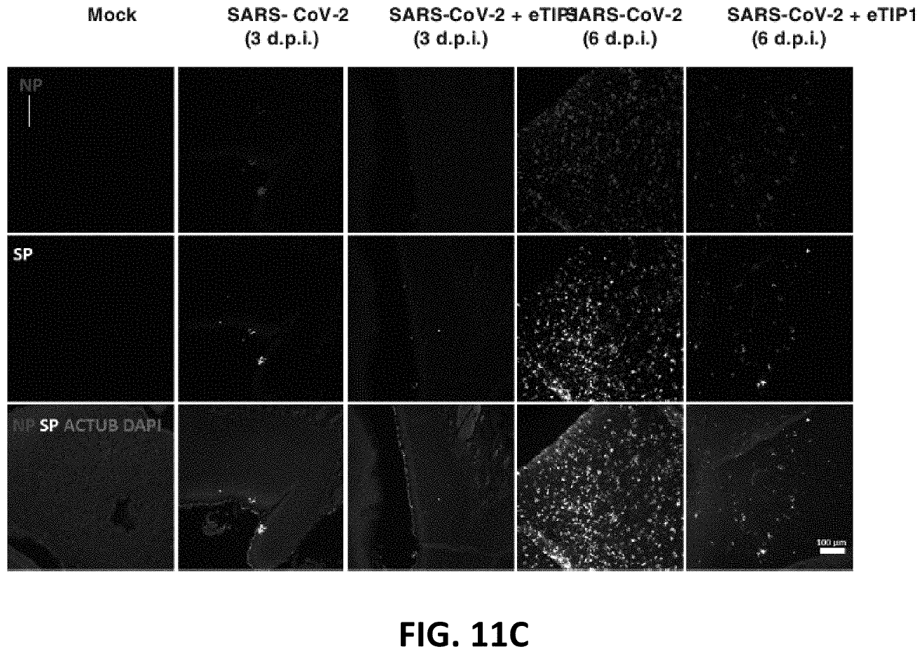

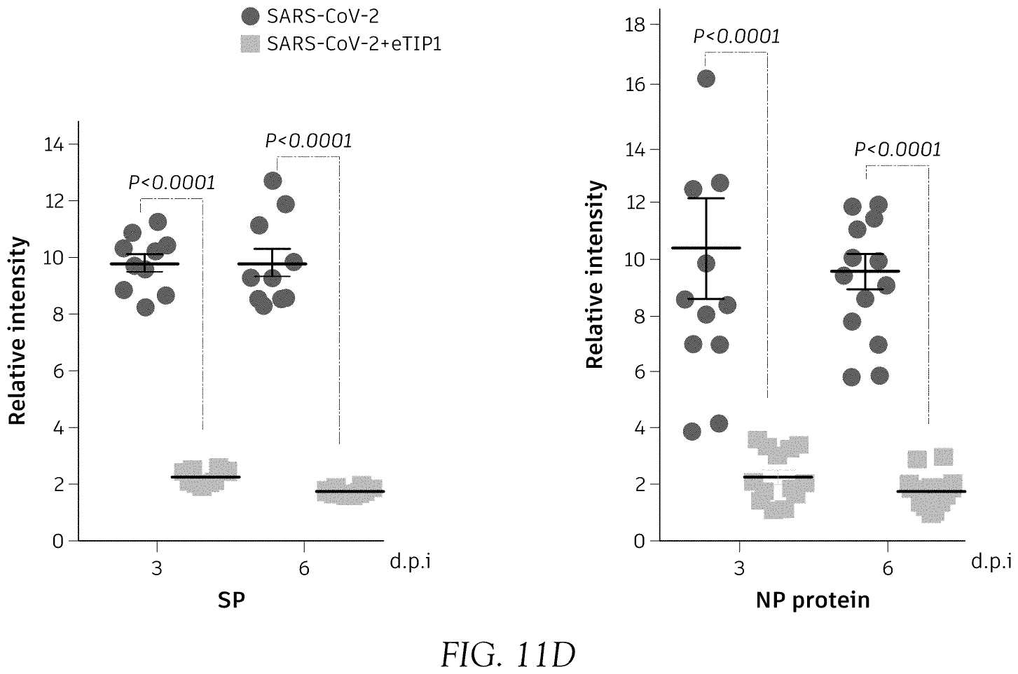

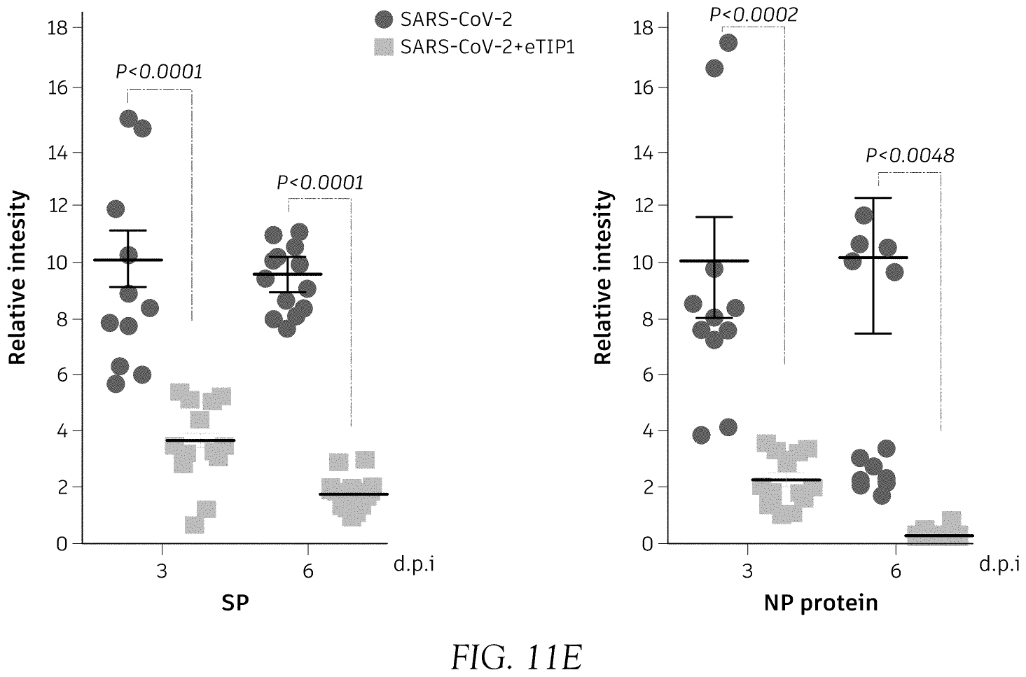

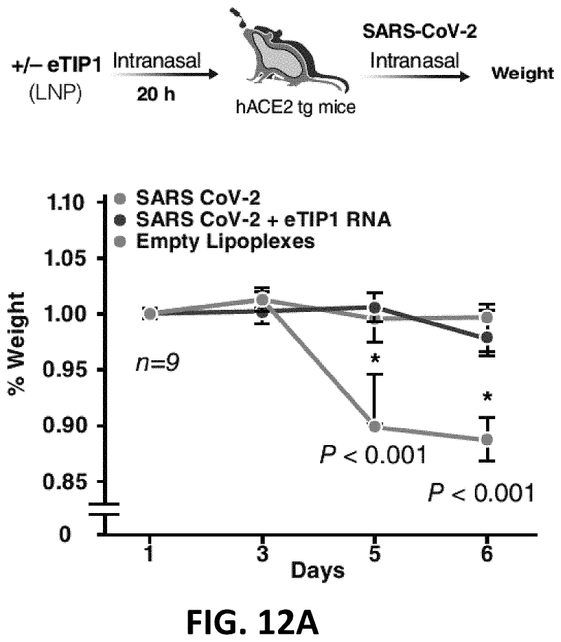

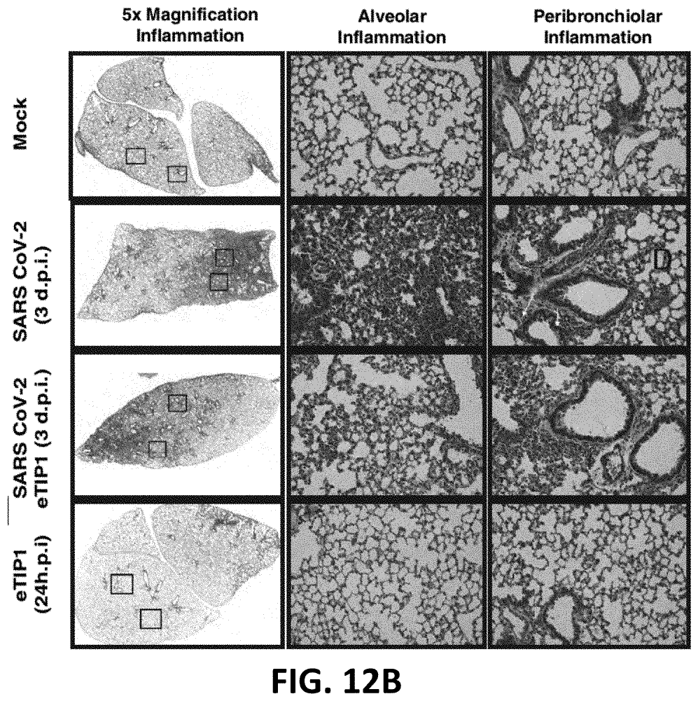

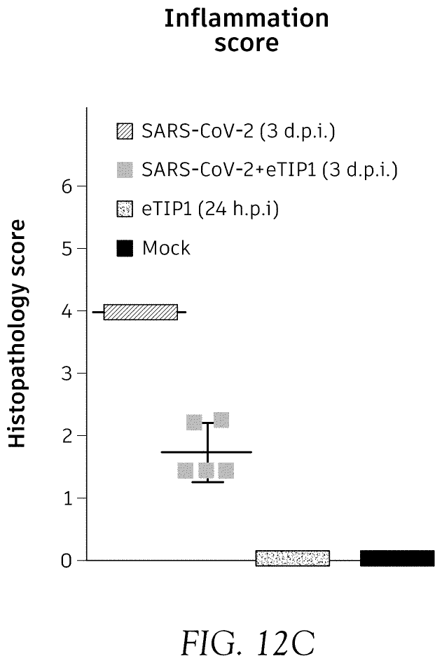

The protective effect of eTIP1 against SARS-CoV-2 after one intranasal administration can provide a powerful prophylactic and therapeutic weapon to combat the ongoing COVID-19 pandemic and future respiratory diseases, including influenza and the common cold, as well as other enterovirus diseases. In the case of COVID, eTIPI can thwart the induction of a proinflammatory response by SARS-CoV-2, thereby preventing damage to the lung and brain (FIGS. 11A-11E). The cytokines induced are not proinflammatory but antiviral (FIG. 9A and FIG. 10A). Thus, eTIP1 both blocks SARS-CoV-2 replication, an important first step in controlling COVID19, but also redirects the host response to SARS infection from an inflammatory to an effective antiviral response, which should increase the therapeutic protection from disease. Indeed, eTIP1 administration to SARS infected animals can protect from disease and recruits lymphoid cells in the lung (FIG. 9B) but blocks production of proinflammatory cytokines (FIGS. 12B-12C). Thus, eTIP1 can mediate antiviral immunity and prevent inflammation.

Taken together, the experimental data described herein have demonstrated the concept that the poliovirus eTIP of the disclosure can inhibit different strains of wild-type enterovirus, including influenza virus in infected animals, and that eTIP is an effective antiviral approach that harnesses the natural process to fight infection in a balanced, safe, and controlled manner, utilizing the regulatory circuits that evolved to ensure that immune system provide protection without causing disease. In these studies, replication of eTIP is important for protection. The experimental data described herein further demonstrate that eTIP can protect from respiratory infection with a highly pathogenic virus, and importantly eTIP can be used both as a preventive and therapeutic strategy to protect against a diverse group of human pathogens. Without being bound to any particular theory, the effect on immune competent mice can be attributed to stimulation of the innate immune responses. Antiviral therapy provided herein can offer greater benefits and safety over other conventional pharmaceutical-based therapies and can transform the approach to combat COVID and emerging and/or re-emerging viral threats.

### Compositions of the Disclosure

### A. Nucleic Acid Constructs

In outlined above, one aspect of the disclosure relates to novel nucleic acid constructs including a nucleic acid sequence encoding a modified enterovirus genome. For example, a modified enterovirus genome can include deletion(s), substitution(s), and/or insertion(s) in one or more of the genomic regions of the parent enterovirus genome.

Non-limiting exemplary embodiments of the nucleic acid constructs of the disclosure can include one or more of the following features. In some embodiments, the modified enterovirus genome is devoid of at least a portion of the nucleic acid sequence encoding viral structural proteins. In some embodiments, the modified enterovirus genome is devoid of at least a portion of the sequence encoding one or more of structural proteins VP1, VP2, VP3, and VP4. In some embodiments, the modified enterovirus genome is devoid of a portion of or the entire sequence encoding VP1. In some embodiments, the modified enterovirus genome is devoid of a portion of or the entire sequence encoding VP2. In some embodiments, the modified enterovirus genome is devoid of a portion of or the entire sequence encoding VP3. In some embodiments, the modified enterovirus genome is devoid of a portion of or the entire sequence encoding VP4. In some embodiments, the modified enterovirus genome is devoid of a portion of or the entire sequence encoding a combination of VP1, VP2, VP3, and VP4.

In some embodiments, the modified enterovirus genome or replicon RNA is devoid of a substantial portion of the nucleic acid sequence encoding viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul SF et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full length nucleic acid sequence. The present disclosure provides nucleic acid molecules and constructs which are devoid of partial or complete nucleic acid sequences encoding one or more viral structural proteins. The skilled artisan, having the benefit of the sequences as disclosed herein, can readily use all or a substantial portion of the disclosed sequences for purposes known to those skilled in this art. Accordingly, the present application comprises the complete sequences as disclosed herein, e.g., those set forth in the accompanying Sequence Listing, as well as substantial portions of those sequences as defined above.

In some embodiments, the modified enterovirus genome is devoid of the entire sequence encoding viral structural proteins, e.g., the modified enterovirus genome comprises no nucleic acid sequence encoding viral structural proteins.

In some embodiments, the modified enterovirus genome is derived from a virus belonging to a Rhinovirus species selected from the group consisting of Rhinovirus A, Rhinovirus B, and Rhinovirus C. In some embodiments, the modified enterovirus genome is derived from a virus belonging to an Enterovirus species selected from the group consisting of Enterovirus A, Enterovirus B, Enterovirus C, Enterovirus D, Enterovirus E, Enterovirus F, Enterovirus G, Enterovirus H, Enterovirus I, Enterovirus J, Enterovirus K, and Enterovirus L. In some embodiments, the modified enterovirus genome is derived from a poliovirus of the Enterovirus C species. In some embodiments, the modified enterovirus genome is derived from a poliovirus serotype selected from the group consisting of PV1, PV2, and PV3. In some embodiments, the modified poliovirus genome or replicon RNA is derived from poliovirus type 1 (PV1).

In some embodiments, the nucleic acid sequence encoding the modified poliovirus genome has at least 80%, at least 90%, at least 95%, at least 96%, at least 97%, at least 98%, at least 99%, or 100% sequence identity to the nucleic acid sequence of SEQ ID NO: 1. Nucleic acid sequences having a high degree of sequence identity (e.g., at least 80%, at least 85%, at least 90%, at least 95%, at least 96%, at least 97%, at least 98%, at least 99%, or 100%) to a sequence encoding structural proteins of an enterovirus of interest can be identified and/or isolated by using the sequences identified herein (e.g., SEQ ID NO: 1) or any others as they are known in the art, by genome sequence analysis, hybridization, and/or PCR with degenerate primers or gene-specific primers from sequences identified in the respective enterovirus genome.

The basic techniques for operably linking two or more sequences of DNA together are familiar to the skilled worker, and such methods have been described in a number of texts for standard molecular biological manipulation. The molecular techniques and methods by which these new nucleic acid molecules were constructed and characterized are described more fully in the Examples herein.

In some embodiments of the disclosure, the nucleic acid sequence encoding a modified enterovirus genome is operably linked to a heterologous nucleic acid sequence. In some embodiments, the heterologous nucleic acid sequence comprises a promoter sequence or a coding sequence for a selectable marker. In some embodiments, the nucleic acid sequence encoding a modified enterovirus genome is incorporated into an expression cassette or an expression vector. It will be understood that an expression cassette generally includes a construct of genetic material that contains coding sequences and enough regulatory information to direct proper transcription and/or translation of the coding sequences in a cell, in vivo and/or ex vivo. Generally, the expression cassette may be inserted into a vector for targeting to a desired host cell and/or into an individual. As such, in some embodiments, an expression cassette of the disclosure include a nucleic acid sequence encoding a modified enterovirus genome as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

### B. Recombinant Cells

The nucleic acid constructs of the present disclosure can be introduced into a host cell to produce a recombinant cell containing the nucleic acid molecule. Accordingly, prokaryotic or eukaryotic cells that contain a nucleic acid construct encoding a modified enterovirus genome as described herein are also features of the disclosure. In a related aspect, some embodiments disclosed herein relate to methods of transforming a cell that includes introducing into a host cell, such as an animal cell, a nucleic acid construct as provided herein, and then selecting or screening for a transformed cell. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

In a related aspect, some embodiments disclosed herein relate to recombinant cells, for example, recombinant animal cells that include a nucleic acid construct described herein. The nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome editing techniques such as using guide RNA directed CRISPR/Cas9 or TALEN genome editing. In some embodiments, the nucleic acid construct present in the recombinant host cell as a mini-circle expression vector for a stable or transient expression.

Host cell useful in the present disclosure is one into which a nucleic acid construct as described herein can be introduced. Common host cells are mammalian host cells, such as, for example, HeLa cells (ATCC Accession No. CCL 2), HeLa S3 (ATCC Accession No. CCL 2.2), the African Green Monkey cells designated BSC-40 cells, which are derived from BSC-1 cells (ATCC Accession No. CCL 26), and HEp-2 cells (ATCC Accession No. CCL 23). Because the enterovirus nucleic acid construct is encapsidated prior to serial passage, host cells for such serial passage are generally permissive for enterovirus replication, e.g. poliovirus replication. Cells that are permissive for enterovirus replication are cells that become infected with the modified enterovirus genome, allow viral nucleic acid replication, expression of viral proteins, and formation of progeny virus particles. In vitro, poliovirus causes the host cell to lyse. However, in vivo the poliovirus may not act in a lytic fashion. Non-permissive cells can be adapted to become permissive cells, and such cells are intended to be included in the category of host cells which can be used in this disclosure. For example, the mouse cell line L929, a cell line normally non-permissive for poliovirus replication, has been adapted to be permissive for poliovirus replication by transfection with the gene encoding the poliovirus receptor. More information in this regard can be found in, e.g., Mendelsohn, C. L. et al. (1989) Cell 56:855-865; Mendelsohn, C. L. et al. (1986) Proc. Natl. Acad Sci. USA 83:7845-7849.

In some embodiments, the recombinant cell is a prokaryotic cell. In some embodiments, the recombinant cell is a eukaryotic cell. In some embodiments, the cell is in vivo. In some embodiments, the cell is ex vivo. In some embodiments, the cell is in vitro. In some embodiments, the recombinant cell is an animal cell. In some embodiments, the animal cell is a mammalian cell. In some embodiments, the animal cell is a human cell. In some embodiments, the cell is a non-human primate cell.

Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Enterovirus Defective Inter Fering Particles of the Disclosure

In one aspect of the disclosure, provided herein are methods for producing a defective interfering (DI) particle of enterovirus. In some embodiments, the methods include: a) providing a host cell engineered to express enterovirus structural proteins or portions thereof, b) transfecting the provided host cell with a nucleic acid construct of the disclosure; and c) culturing the transfected host cell under conditions for production of a DIP of enterovirus comprising the nucleic acid construct encapsidated by the expressed enterovirus structural proteins or portion thereof.

Non-limiting exemplary embodiments of the methods for producing a DI particle of enterovirus disclosed herein can include one or more of the following features. In some embodiments, the host cell has been previously infected with an enterovirus with a complete enterovirus genome including coding sequence for structural proteins (sometimes referred to as a “helper virus”). In some embodiments, at least one of the enterovirus structural proteins expressed by the host cell is heterologous relative to the enterovirus genome encoded by the nucleic acid construct. For example, in some embodiments, at least one of the expressed enterovirus structural proteins is derived from an enterovirus species that is different from the enterovirus species from which the enterovirus genome encoded by the nucleic acid construct is derived. In some embodiments, at least a portion of the sequence encoding VP1, VP2, VP3, and/or VP4 expressed by the host cell is heterologous relative to the enterovirus genome encoded by the nucleic acid construct. In some embodiments, all of the enterovirus structural proteins expressed by the host cell are from the same enterovirus species that the modified enterovirus genome is derived from.

In some embodiments, the methods for producing enteroviral DIPs described herein further include harvesting and/or purifying the produced DIP. Methods, approaches, protocols, and systems suitable for the harvest and/or purification of enteroviral DIPs are known in the art. For example, enteroviral DIPs produced in accordance with the present disclosure can be harvested by one or more of methods, such as, centrifugation, filtration, PEG precipitation, or passing through columns of anti-surface antibodies. More information in this regard can be found in, for example, U.S. Pat. No. 5,980,901 and PCT Patent Publication No. WO2007135420 A2. Accordingly, DIPs produced by the methods described herein are also within the scope of the present disclosure.

In a related aspect, provided herein are enteroviral DIPs include a nucleic acid construct of the disclosure. In some embodiments, the enteroviral DI particles include a nucleic acid construct of the disclosure, which is encapsidated by heterologous capsid structural proteins, e.g., capsid structural proteins derived (e.g, expressed) from a different viral genome. In some embodiments, the enteroviral DI particles include a nucleic acid construct of the disclosure encapsidated by capsid structural proteins derived (e.g, expressed) from a homologous helper wild-type virus.

### D. Pharmaceutical Compositions

The nucleic acid constructs, defective interfering particles (DIPs), recombinant cells, and/or cell cultures of the disclosure can be incorporated into compositions, including pharmaceutical compositions. Such compositions generally include one or more of the nucleic acid constructs, defective interfering particles (DIPs), recombinant cells, and/or cell cultures as provided and described herein, and a pharmaceutically acceptable excipient, e.g., carrier. In some embodiments, the pharmaceutical compositions of the disclosure are formulated for the prevention, treatment, or management of a health condition such as an immune disease or a viral infection.

In some embodiments, the composition includes a DIP as described herein and a pharmaceutically acceptable excipient. In some embodiments, the composition includes a nucleic acid construct as described herein and a pharmaceutically acceptable excipient. In some embodiments, includes (i) a DIP as described herein; (ii) a nucleic acid construct as described herein; and (iii) a pharmaceutically acceptable excipient.

In some embodiments, the nucleic acid construct and/or the DIP is formulated in a liposome. In some embodiments, the nucleic acid construct and/or the DIP is formulated in a lipid nanoparticle. In some embodiments, the nucleic acid construct and/or the DIP is formulated in a polymer nanoparticle.

In some embodiments, the composition of the disclosure is an immunogenic composition, e.g., a composition that can stimulate an immune response in a subject. In some embodiments, the immunogenic composition of the disclosure is formulated as a vaccine. In some embodiments, the immunogenic composition of the disclosure is formulated as an adjuvant.

In some embodiments, the pharmaceutical composition is formulated for one or more of intranasal administration, transdermal administration, intraperitoneal administration, intramuscular administration, intravenous administration, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the composition is formulated for one or more of intranasal administration, transdermal administration, intramuscular administration, intravenous administration, intraperitoneal administration, oral administration, or intra-cranial administration. In some embodiments, the administered composition results in an increased production of interferon in the subject.

### Methods of the Disclosure

Administration of any one of the therapeutic compositions described herein, e.g., nucleic acid constructs, defective interfering particles (DIPs), recombinant cells, and pharmaceutical compositions, can be used to treat subjects for relevant health conditions, such as immune diseases and microbial infections (e.g., viral infections). In some embodiments, the nucleic acid constructs, DIPs, recombinant cells, and pharmaceutical compositions described herein can be incorporated into therapeutic agents for use in methods of preventing or treating a subject who has, who is suspected of having, or who may be at high risk for developing one or more health conditions, such as autoimmune diseases or viral infections.

Accordingly, in one aspect, provided herein are methods for eliciting an immune response in a subject in need thereof, the method comprises administering to the subject a composition including: (a) a DIP of the disclosure; (b) a nucleic acid construct of the disclosure; (c) a recombinant cell of the disclosure; and/or (d) a pharmaceutical composition of the disclosure.

In another aspect, provided herein are methods for preventing and/or treating a health condition in a subject in need thereof, the methods include prophylactically or therapeutically administering to the subject a composition including: (a) a DIP of the disclosure; (b) a nucleic acid construct of the disclosure; (c) a recombinant cell of the disclosure; and/or (d) a pharmaceutical composition of the disclosure.

In some embodiments, the DIPs of the disclosure can be used in a composition for stimulating a mucosal as well as a systemic immune response. The mucosal immune response is an important immune response because it offers a first line of defense against infectious agents, such as a virus which can enter host cells via mucosal cells. Upon administration of the enterovirus DIPs of the disclosure, the subject will generally respond to the immunizations by producing both anti-enterovirus antibodies. The enterovirus nucleic acid constructs of the disclosure, in either DNA or RNA form, can also be used in a composition for stimulating a systemic and a mucosal immune response in a subject. In some embodiments, administration of the RNA form of the enterovirus nucleic acid constructs is carried out as it generally does not integrate into the host cell genome.

The DIPs and/or the non-encapsidated enterovirus nucleic acid constructs of the disclosure can be administered to a subject in a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject.

As described above, pharmaceutically acceptable carriers suitable for injectable use include sterile aqueous solutions (where water soluble) or dispersions and sterile powders for the extemporaneous preparation of sterile injectable solutions or dispersions. In all cases, the composition must be sterile and must be fluid to the extent that easy syringability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, asorbic acid, thimerosal, and the like.

Sterile injectable solutions can be prepared by incorporating the DIPs in the required mount in an appropriate solvent with one or a combination of ingredients enumerated above, as required, followed by filtered sterilization.

When the DIPs and/or nonencapsidated enterovirus nucleic acid constructs are suitably protected, as described above, they may be orally administered, for example, with an inert diluent or an assimilable edible carrier. The DIPs and/or nonencapsidated enterovirus nucleic acid constructs and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, suspensions, syrups, wafers, and the like.

Recombinant cells that produce the DIPs of the present disclosure can be introduced into a subject, thereby stimulating an immune response to the elements encoded by the enterovirus nucleic acid constructs. In some embodiments, the recombinant cells that are introduced into the subject are first removed from the subject and contacted ex vivo with both the enterovirus nucleic acid constructs. The recombinant cells that produce the DIPs can then be reintroduced into the subject by, for example injection or implantation. Examples of cells that can be modified by this method and injected into a subject include peripheral blood mononuclear cells, such as B cells, T cells, monocytes and macrophages. Other cells, such as cutaneous cells and mucosal cells can be modified and implanted into a subject.

Non-limiting exemplary embodiments of the methods of the disclosure can include one or more of the following features. In some embodiments, the therapeutic compositions described herein, e.g., nucleic acid constructs, defective interfering particles (DIPs), recombinant cells are incorporated into therapeutic compositions for use in methods of preventing or treating a subject who has, who is suspected of having, or who may be at high risk for developing an autoimmune disease.

In some embodiments, the therapeutic compositions described herein, e.g., nucleic acid constructs, defective interfering particles (DIPs), recombinant cells are incorporated into therapeutic compositions for use in methods of preventing or treating a subject who has, who is suspected of having, or who may be at high risk for developing a viral infection. In some embodiments, the infection is a seasonal respiratory viral infection or an acute respiratory viral infection. In some embodiments, the infection is caused by a virus belonging to a species of the Human orthopneumovirus genus, a species of the Enterovirus family, a species of the Coronaviridae family, or a subtype of the Orthomyxoviridae family. In some embodiments, the orthomyxovirus is an influenza A virus or a Parainfluenza virus. Non-limiting examples of influenza A virus subtypes suitable for the methods described herein include influenza A subtypes H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

Examples of parainfluenza virus subtypes suitable for the methods of the disclosure include, but are not limited to, parainfluenza subtypes HPIV-1, HPIV-2, HPIV-3, and HPIV-4. In some embodiments, the coronavirus is β-CoV severe acute respiratory syndrome coronavirus (SARS-CoV), severe acute respiratory syndrome coronavirus 2 (SARS-CoV-2). In some embodiments, the coronavirus β-CoV infection is associated with one or more Sarbecovirus subgenera. Examples of suitable Sarbecovirus subgenera include, but are not limited to, severe acute respiratory syndrome coronavirus SARSr-CoV (which includes all its strains such as SARS-CoV, SARS-CoV-2, and Bat SL-CoV-WIV1), subgenus Merbecovirus consisting of Tylonycteris bat coronavirus HKU4 (BtCoV-HKU4), Pipistrellus bat coronavirus HKU5 (BtCoV-HKU5), and Middle East respiratory syndrome-related coronavirus MERS-CoV (which includes the species HCoV-229E, HCoV-NL63, HCoV-OC43, HCoV-HKU1). In some embodiments, the viral infection is associated with a human-infecting coronaviruses such as SARS-1, SARS-2, MERS, and endemic coronaviruses 229E, NL63, OC43, and HKU1. In some embodiments, the viral infection is associated with SARS-CoV-2. In some embodiments, the human orthomyxovirus is a human respiratory syncytial virus (HRSV). In some embodiments, the HRSV is associated with subtype A and/or subtype B.

In some embodiments of the disclosure, the viral infection is an enteroviral infection. In some embodiments, the enteroviral infection is associated with one or more Enterovirus species selected from the group consisting of Enterovirus A species, Enterovirus B species, Enterovirus C species, Enterovirus D species, Enterovirus E species, Enterovirus F species, Enterovirus G species, Enterovirus H species, Enterovirus I species, Enterovirus J species, Enterovirus K species, and Enterovirus L species. In some embodiments of the disclosure, the viral infection is a rhinoviral infection. In some embodiments, the rhinoviral infection is associated with one or more Rhinovirus species selected from the group consisting of rhinovirus A species, rhinovirus B species, and rhinovirus C species. In some embodiments, the viral infection is associated with one or more of poliovirus type 1 (PV1), poliovirus type 3 (PV3), coxsackievirus A2, coxsackievirus A4, coxsackievirus A16, coxsackievirus B1, coxsackievirus B3 (CV-B3), coxsackievirus B6, Parechovirus (echovirus), enterovirus A71 (EV-A71), enterovirus D68 (EV-D68), rhinovirus HRV16, and rhinovirus HRV1B.

### Additional Therapies

In some embodiments, any one of the compositions disclosed herein, e.g., nucleic acid constructs, defective interfering particles (DIPs), recombinant cells, cell cultures, and/or pharmaceutical compositions as described herein can be administered to the subject individually as a single therapy (monotherapy). In addition or alternatively, in some embodiments of the disclosure, the nucleic acid constructs, defective interfering particles (DIPs), recombinant cells, cell cultures, and/or pharmaceutical compositions as described herein can be administered to the subject as a first therapy in combination with at least one (e.g., at least one, two, three, four, or five) additional therapies (e.g., second therapy). Suitable therapies to be administered in combination with the compositions of the disclosure include, but are not limited to chemotherapy, radiotherapy, immunotherapy, hormonal therapy, toxin therapy, targeted therapy, and surgery. Administration “in combination with” one or more additional therapies includes simultaneous (concurrent) and consecutive administration in any order. Accordingly, in some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are various kits for the practice of a method described herein. In particular, some embodiments of the disclosure provide kits for eliciting an immune response in a subject. Some other embodiments relate to kits for the prevention of a health condition in a subject in need thereof. Some other embodiments relate to kits for methods of treating a health condition in a subject in need thereof. For example, provided herein, in some embodiments, are kits that include one or more of the DIPs, nucleic acids, recombinant cells, cell cultures, or pharmaceutical compositions as provided and described herein, as well as written instructions for making and using the same.

In some embodiments, the kits of the disclosure further include one or more means useful for the administration of any one of the provided nucleic acid constructs, DIPs, recombinant cells, cell cultures, or pharmaceutical compositions to a subject. For example, in some embodiments, the kits of the disclosure further include one or more syringes (including pre-filled syringes) and/or catheters (including pre-filled syringes) used to administer any one of the provided DIPs, nucleic acid constructs, recombinant cells, cell cultures, or pharmaceutical compositions to a subject. In some embodiments, a kit can have one or more additional therapeutic agents that can be administered simultaneously or sequentially with the other kit components for a desired purpose, e.g., for eliciting an immune response in a subject, for diagnosing, preventing, or treating a condition in a subject in need thereof.

Any of the above-described kits can further include one or more additional reagents, where such additional reagents can be selected from: dilution buffers; reconstitution solutions, wash buffers, control reagents, control expression vectors, negative controls, positive controls, reagents suitable for in vitro production of the DIPs, nucleic acid constructs, recombinant cells, or pharmaceutical compositions of the disclosure.

In some embodiments, the components of a kit can be in separate containers. In some other embodiments, the components of a kit can be combined in a single container. For example, in some embodiments of the disclosure, the kit includes one or more of the r the provided nucleic acid constructs, DIPs, recombinant cells, cell cultures, or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, N.Y.: Cold Spring Harbor Laboratory and Sambrook, J., & Russel, D. W. (2001). Molecular Cloning: A Laboratory Manual (3rd ed.). Cold Spring Harbor, N.Y.: Cold Spring Harbor Laboratory (jointly referred to herein as “Sambrook”); Ausubel, F. M. (1987). Current Protocols in Molecular Biology. New York, N.Y.: Wiley (including supplements through 2014); Bollag, D. M. et al. (1996). Protein Methods. New York, N.Y.: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, Calif.: Academic Press; Lefkovits, I. (1997). The Immunology Methods Manual: The Comprehensive Sourcebook of Techniques. San Diego, Calif.: Academic Press; Doyle, A. et al. (1998). Cell and Tissue Culture: Laboratory Procedures in Biotechnology. New York, N.Y.: Wiley; Mullis, K. B., Ferre, F. & Gibbs, R. (1994). PCR: The Polymerase Chain Reaction. Boston: Birkhauser Publisher; Greenfield, E. A. (2014). Antibodies: A Laboratory Manual (2nd ed.). New York, N.Y.: Cold Spring Harbor Laboratory Press; Beaucage, S. L. et al. (2000). Current Protocols in Nucleic Acid Chemistry. New York, N.Y.: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

### Example 1. Cells, Plasmids and Virus

Hela S3 cells (ATCC, CCL-2.2), A549 cells (ATCC® CCL-185™), primary embryo fibroblast cells (MEFs), Calu-3 cells (ATCC® HTB-55), and A549-Ace2 cells were cultured in DMEM high glucose/F12 medium supplemented with 10% fetal bovine serum (Sigma) and 1× penicillin/streptomycin/glutamine (100×PSG, Gibco). For packaging cell line, Hela S3 cells stably overexpressing poliovirus P1 gene (Hela S3/P1) were cultured in DMEM high glucose/F12 medium supplemented with 10% fetal bovine serum (Sigma) and 1× penicillin/streptomycin/glutamine (100×PSG, Gibco) plus 0.015% Zeocin (Invitrogen). RD cells (ATCC, CCL-136™) were cultured in DMEM/H21 medium supplemented with 10% fetal bovine serum (Sigma) and 1× penicillin/streptomycin/glutamine (100×PSG, Gibco). A549-ACE2 cells were stable expression under-CMV promoter, a gift form Peter Jackson lab (Stanford University). Hela H1 cells or RD cells (ATCC, CCL-136™) were cultured in DMEM/H21 medium supplemented with 10% fetal bovine serum (Sigma) and 1× penicillin/streptomycin/glutamine (100×PSG, Gibco).

In this study, the Mahoney strain of poliovirus Type 1 (PV1) was used as wild-type PV1 virus. In some embodiments, defective interference particle (eTIP) were lacking the P1 genes of PV1 virus and replaced by a GFP-Venus gene. Plasmid prib(+)XpA was digested by Nrul and SnaBI (New England Biolabs) and ligated to produce prib(+)XpA, which lacked the poliovirus capsid-encoding region from 1175 to 2956 prib(+)XpA16. EV-D68 virus were made by a plasmid (PUC57-EV-D68-49131), linearized and in vitro transcribed (IVT) as PV1. Coxsackie virus B3 (CVB3) was the Nancy strain, the type 3 poliovirus (PV3), Leon strain. Influenza A virus strain A/PR/8/34 (H1N1) was a gift from Professor Christopher Byron Brooke (University of Illinois).

To amplify SARS-CoV-2, A549-Ace2 cells were infected with SARS-CoV-2 clinical isolate (Spike D614G (Deng et al., 2021), a clinical SARS-COV-2 isolate from UCSF patient) with MOI ˜0.05 in MEM medium supplemented with 2% FBS and penicillin/streptavidin (Gibco). Three days after infection, the medium was collected and cleared from cell debris by centrifugation at 3000 g for 10 min at 4° C. The virus titers were measured by plaque assay on Vero-E6 cells (see, e.g., Example 12).

### Example 2.In Vitro Transcription (IVT) RNAs, Transfection and eTIP Production

To generate viruses and eTIP, T7 polymerase was used to generate in vitro transcribed (IVT) viral RNA derived from corresponding linearized prib(+)XpA Mahoney or eTIP plasmid by Apal. The resulting 10 μg IVT RNA of PV1 were electroporated into 8×10⁶ Hela S3 cells. And IVT RNAs of eTIP were electroporated into 8×10⁶ packing cells line. Monolayer of Hela S3 or packaging cells was trypsinized and washed three times in D-PBS. Cells were resuspended in D-PBS and the number of cells were counted on a hemo-cytometer, followed by adjusting the concentration to 10⁷ cells per ml. 800 μl of cells and 10 μg IVT RNAs were transferred into a chilled 4-mm electroporation cuvette and incubated 20 minutes on ice. Cells were electroporated with IVT RNAs (voltage=200 V, capacitance=1000 μF) using Gene Pulser I (Bio-Rad), and recovered in 8 ml pre-warmed medium. Viruses and eTIP were harvested at around 24 hours (or total CPE) to generate P0 virus or eTIP stocks. P0 virus stock were amplified once in cultured Hela S3 in 2% serum media at M.O.I 0.2 to generate a passage 1 (P1) stocks. P0 eTIP stocks were amplified once in cultured packaging cell line in 2% serum media at M.O.I˜ 0.2 to generate a passage 1 (P1) stocks. Influenza A virus strain A/PR/8/34 (H1N1) is a gift from Professor Christopher Brooke.

### Example 3. Titration of Virus and eTIP Samples

Monolayers of Hela S3 cells in 6-well plates were infected with 250 μl of serially diluted virus samples then incubated at 37° C. for about 30 minutes, then 1% agarose overlay were added on the top. For titer eTIP, Hela S3 cells were grown in 48-wells plate. On the following day, 100 μl, 10 folds serially diluted eTIP samples were added to each well. After incubation for 1 hours, 400 μl of regular medium were add into each well. At 8-9 hours post-infection, GFP-positive cells were counted as the eTIP titers and represent IU/ml. To measure the EV-D68 infected samples, TCID50 (median tissue culture infectious dose) were performed on RD cells. RD cells were seed to 96 wells plate in 2% FBS DMEM/H21 medium with 10⁴ cells per well one day before performing the TCID50.

### Example 4. Design of Primers and TaqMan® Probes (Droplet PCR)

Primers and Taqman probes for droplet digital PCR assay were designed with PrimerQuest™ Tool (Integrated DNA Technologies). The primers and probe for PV1 genomes were 5′-CCACATACAGACGATCCCATAC-3′ (SEQ ID NO: 2), 5′-CTGCCCAGTGTGTGTAGTAAT-3′ (SEQ ID NO: 3), and 5′-6-FAM™-TCTGCCTGTCACTCTCTCCAGCTT-3′-BHQ-1@ (SEQ ID NO: 4). The primers and probe for eTIP1 genomes were 5′-GACAGCGAAGCCAATCCA-3′ (SEQ ID NO: 5), 5′-CCATGTGTAGTCGTCCCATTT-3′ (SEQ ID NO: 6), and 5′-HEX-ACGAAAGAG/ZEN™/TCGGTACCACCAGGC-3′-IABkFQ (SEQ ID NO: 7). Information regarding the fluorophores or dyes FAM™, BHQ-1@, HEX (Hexachlorofluorescein), ZEN™, and IABkFQ (Iowa Black FQ quencher) coupled to the above primers and probes can be found at, e.g., Intergated DNA Technologies website (idtdna.com). Droplet digital PCR assay: 2 μl of serially diluted cDNA samples was mixed with 10 μl of 2× ddPCR super-mix for probes (Bio-Rad), 1 μl of 20× PV1 or eTIP primers/probe, 1 μl of 20× eTIP primers/probe, and 6 μl of nuclease-free water. 20 μl reaction mix of each sample was dispensed into the droplet generator cartridge, followed by droplet production with QX100 droplet generator (Bio-Rad). Then PCR was performed on a thermal cycler using the following parameters: 1 cycle of 10 minutes at 95° C. and 60° C. of 1 minute for 40× cycles. Then the PCR product were read and calculated by QX100 droplet reader.

### Example 5. Virus 2Growth Curve of PV1 and Co-Infected Replication Kinetics in Hela and MEFs

2.5×10⁵ mouse MEFs or Hela S3 cells were seeded in 24-well plates. On the following day, cells were washed twice with PBS and were infected with virus in 200p1, 2% serum media at M.O.I=0.1 with PV1 alone or co-infected with mixed PV1+DI (at ratio: 1:10, or 1:100) or with eTIP alone at MOI=1 or 10 (three replicate wells were used for each virus at each time point). Following an hour incubation at 37° C., each well was washed twice with PBS, and cells were covered with fresh complete media. At each indicated time point, the corresponding plate was frozen at −80° C. Following three freeze-thaw cycles of the plates, standard plaque assays were performed on monolayer Hela S3 cells grown in a 6-wells plate (˜10⁶ Hela S3 cells per well).

### Example 6. Interferon Production Measured by ELISA and qRT-PCR

Murine embryos fibroblast cells (MEFs) were isolated from 13-14 days old embryos. MEF monolayers were seeded at 2.5×10⁵ cells/well in 24-well plates, infected by PV1 poliovirus at MOI=0.1 or mixed PV1 with eTIP at different ratio: 1:1, 1:10, 1:20,1:100. At indicated time points, supernatant was collected to measure interferon induction by ELISA (VeriKine-HS Murine Interferon Beta Serum ELISA Kit, PBL Assay Science). Virus titer were calculated by plaque assay in Hela S3 cells, eTIP titers were measured by GFP positive cells.

### Example 7. Virus Growth Curve of PV1 or Other Wildtype Viruses and Co-Infected Replication Kinetics in Cell Culture Models

2.5×10⁵ HelaS3 cells were seeded in 24-well plates. On the following day, cells were washed twice with PBS and were infected with virus in 200 μl, 2% serum media at M.O.I=0.1 with virus alone or co-infected with mixed virus+eTIP1 (at ratio: 1:10, 1:20, 1:50, 1:100) or with eTIP1 alone at MOI 5 (three replicate wells were used for each virus at each time point). Following an hour incubation at 37° C., each well was washed twice with PBS, and cells were covered with fresh complete media. At each indicated time point, the corresponding plate was frozen at −80° C. Following three freeze-thaw cycles of the plates, standard plaque assays were performed on monolayer HeLaS3 cells grown in a 6-wells plate (˜106 HeLaS3 cells per well).

Or TCID50 for EV-D68 on RD cells.

### Example 8. Purification of the eTIP Particles

Packaging cell lines generating eTIPs (500 ml) was harvested with 0.5% NP-40, and the sample was stored at −80° C. For virus purification, the sample was subjected to three freeze-thaw cycles. For virus precipitation, PEG 8000 was added to a final concentration of 10% and stored overnight at 4° C. The precipitated sample was pelleted by spinning at 3,500 g for 1 hour. The pellet was suspended in 10 ml EB-buffer (50 mM Tris pH 8.0, 300 mM NaCl, 5 mM MgCl₂, 0.5% NP-40) and centrifuged at 3,500 g for 30 minutes at 4° C. to remove cell debris and insoluble materials. The soluble fraction containing eTIPs in the supernatant was overlayed on a 2 ml 30% sucrose cushion in EB-buffer at 105,000 g for 3 hours at 4° C. The pellet was suspended in EB-buffer and centrifuged at 12,000 g for 30 minutes at 4° C. to remove insoluble material. The soluble fraction containing eTIPs was then laid on the top of a 15-45% sucrose gradient in EB-buffer and centrifuged at 105,000 g for 3 hours at 4° C. Fractions of 1 ml size from top of the gradient was collected containing eTIPs. Two fractions from top were pooled together, and sucrose in the sample was removed using a spin desalting column (Zebra; Pierce) and buffer exchanged with PBS. eTIPs in PBS were then concentrated using Amicon ultra device with 100 kDa MWCO. Purity and integrity of the eTIPs were evaluated by SDS-PAGE and silver staining. Negative stain and electro-microscopy were used on the particle. Fractions #5-6 were combined and used to inoculation into mice.

### Example 9. Infection of Susceptible Mice

This Examples describes the results of experiments performed to investigate infection of susceptible mice by following protocols approved by the UCSF Institutional Animal Care and Use Committee (IACUC) for mouse studies. In these experiments, 5 to 6-weeks-old Tg21PVR (Tg21) or 6 to 8-weeks-old Tg21 PVR interferon a/I3 receptor knockout (IFNAR^{-/-}) both male and female mice were used and infected under anesthesia. Tg21 and IFNAR^{-/-} were kindly provided by Professor Julie Pfeiffer of the University of Texas Southwestern Medical Center, and originally were generated by Dr. Satoshi Koike of Tokyo Metropolitan Institute for Neuroscience (TOKYO).

For mice survival studies, mice were injected by (i) intra-muscular (I.M., 50 μl of inoculum administered in each hind leg), (ii) intra-peritoneal injection (IP., 100 μl per mouse), (iii) intra-nasal injection (I.N., 20-35 μl per mouse), or (iv) intra-cranial injection (I.C., 20 μl per mouse) with serial dilutions of each virus, respectively (10 mice per group). If UV- treatment was involved, then the eTIPs were UVed for 2 hours. For the influenza H1N1 (PR8 strain) experiment, the mouse were weighted daily. Mice were monitored twice daily for the onset of paralysis and were euthanized when death was imminent.

For protection study, Tg21 mice (8-10 mice per group) were each injected with viral supernatant 10⁷ PFU of PV1 alone or with eTIP ratio at 1:1, 1:10 per mouse by I.P. route or 1:20 by I.N. route (PV1 was 2×10⁵ PFU per mouse), viral supernatant of 200 PFU PV1 alone or with eTIP at different ratios: 1:10, 1:100, 1:250, 1:5000, 1:7500 by I.M. route. For protection study, 200 PFU PV1 virus were inoculated by I.M. route to IFNAR-, then at Day 3, Day 4 and Day 5 post-infection, 2×10⁵ IU eTIP were inoculated by intra-cranial (I.C.) route respectively (4 mice per group). For tissue distribution studies, Tg21 mice (3 to 5 mice per group) were each injected with 3×10⁵ PFU of PV1 virus per mouse by I.N. route, IFNAR^{-/-} mice were inoculated by I.M. route with 200 PFU PV1 alone or with eTIP (PV1: eTIP at ratio=1:5000) per mouse (3 mice per group). Half of the organs were collected from infected mice and homogenized in 1 ml serum-free media. Viral supernatants were collected from the tissue homogenates, following three freeze-thaw cycles, and centrifuged at 5,000×g for 10 min in a bench top centrifuge at 4° C. Regular plaque assays were performed on Hela S3 cells to titer viral supernatants from tissues.

For the pre-treat experiments, Tg21 mice were each inoculated with 6×10⁶ IU eTIP in PBS by intranasally. At 24 hours and 48 hours inoculation, 2×10⁵ PFU PV1 was inoculated into Tg21 mice. At 48 hours pre-treatment inoculation, for the therapeutics experiments, Tg21 mice were each injected with 2×10⁵ PFU PV1 by intranasally at Day 0, then inoculated with 6×10⁶ IU eTIP in PBS by intranasally daily from Day 1 to Day 5.

For tissue distribution studies, The IFNAR^{-/-} mice were inoculated by I.M. route with 200 PFU PV1 alone or with eTIP (PV1: eTIP1 at ratio=1:5000) per mouse (3 mice per group). Mice were euthanized with CO₂, muscle, spleen, spinal cord were collected and at 1, 3, 6 days post-infection. The tissues were homogenized in 1 ml Trizol reagents (Ambion). Total RNAs were extracted and treated with DNasel (NEB). RT-qPCR were formed as droplet qPCR section.

### Example 10. Flow Cytometry Analysis of the Immune Cells from Lung

This Examples describes the results of prophylactic experiments for poliovirus challenging. In these experiments, 6 weeks old Tg21PVR mice were inoculated with 6×10⁶ IU eTIP1 particles in PBS by intranasally. At 48 hours inoculation, mice were euthanized with CO₂ and perfusion with PBS. The full lungs were removed, washed twice with PBS and RPMI-1640 (Gibco). The whole lungs were cut as small pieces and put with 4 ml digestion buffer (RPMI-1640+10 mg/ml Collagen D+10 mg/ml DNasel, 5% FBS) for 30 mins. The tissues were minced with 10-mL syringe, and pass through with 70 μM cell- strainer. Cells were then spun down and washed twice with D-PBS at 650 g at 4° C. for 5 mins. The red cells were lysed with ACS lysis buffer (Thermo fisher, Cat #A1049201) for 2 mins. Cells were then spun down and washed twice with D-PBS at 650 g at 4° C. for 5 mins, Collagen D (Worthington Biochemical, Cat #LS004210), DNase1(Sigma-Aldrich, Cat #11284932001).

Cells were stained with Trypan blue and counted. 10⁶ cells were used for full antibodies panel staining with antibodies as shown in Table 1. 10⁵ cells were used for live/dead staining for 15 mins (eFluor 506 Fix Viability, eBioscience, Cat #65-0866-14), single antibody or unstaining cells or all antibodies. Mouse Fc block (BD Pharmingen, Cat #553141) were diluted in D-PBS. Antibodies was diluted in 1:100 in BD brilliant stain buffer (BD Biosciences, Cat #563794). After all steps staining were completed, cells were fixed with 2% PFA for 30 mins at 4° C., then washed with D-PBS for two times. Samples were resuspended in 300 μl FACS buffer (D-PBS+0.2% BSA+2 mM EDTA). Samples were run in the BD Aries 3, and analyzed with Flowjo software.

TABLE 1
Antibody panel

| | Antibody | Fluorophore | |
|---|---|---|---|
| 1 | CD45 | Alexa_fluor 700 | BioLegend, Cat #103128 |
| 2 | CD11c | APC-Cy7 | BioLegend, Cat #117324 |
| 3 | Singlec-F | PE | BD Biosciences, Cat #552126 |
| 4 | Ly6G | PE-Cy7 | BioLegend, Cat #127618 |
| 5 | Ly6C | Brilliant violet 605 | BioLegend, Cat #128036 |
| 6 | CD11b | Brilliant violet 711 | BioLegend, Cat #101242 |
| 7 | CD103 | APC | BioLegend, Cat #121414 |
| 8 | MHC-2 | Pacific Blue | Biole BioLegend, Cat #107632 |
| 9 | CD45R | PE-Cy5 | BioLegend, Cat #103210 |
| 10 | CD317 | Alexa_fluor 488 | BioLegend, Cat #127012 |
| 11 | Live/dead | eFluor 506 Fix | eBioscience, Cat #65-0866-14 |
| | | Viability | |

### Example 11. Neutralization Antibody Assay (Nab)

Sera was diluted with DMEM at a two folds series dilution (50 μl) then was incubated 200PFU in 50 μl WT virions for 2 hours at 37° C. 100 μl sera and virus then transfer to a 96 wells-plate which contains 5×10³ cells in 100 μL per well. After 7 days, CPE was checked where the cells without CPE were count as the NAb generation.

### Example 12. Experiments with SARS-CoV-2

This Example describes general methods and materials used in experiments performed with severe acute respiratory syndrome coronavirus 2 (SARS-CoV-2). Generally, SARS-CoV-2 cell culture and animals studies were performed in the Biosafety level 3 (BSL-3).

SARS-CoV-2 Virus Propagation and Infection:

African green monkey kidney Vero-E6 cell line (ATCC #1586) and Calu-3 cells(ATCC® HTB-55) was obtained from American Type Culture Collection (ATCC #1586) and maintained in Minimum Essential Medium (MEM, Gibco Invitrogen) supplemented with 10% fetal bovine serum (FBS, Gibco Invitrogen), 1% Penicillin-Streptomycin-Glutamine (Gibco Invitrogen) at 37° C. in a humidified 5% CO2 incubator. A clinical isolate of SARS-CoV-2 (USA-WA1/2020, BEI Cat No: NR-52281) was propagated in Vero E6 cells and A549-ACE2 cells. Viral titer was quantified with plaque assay. All the infections in the context of SARS-CoV-2 were performed at biosafety level-3 (BSL-3).

For prophylactic and coinfection experiments, ˜70% monolayers of Calu-3 cells (1×10⁵ cells/well 720 in 24-well plates) were pretreated with eTIPI particles with MOI=5 for 5 hours (pretreatment), then infected with SARS-CoV-2 (MOI=0.1) for 1 hour at 37° C., the virus mixture was removed, cells were further cultured with medium. At indication time-point 16, 24, 36, 48 hpi (hour post infection), supernatants were collected and viral titers of supernatant were detected with plaque assay.

For co-infection experiment, Calu-3 cells were infected with SARS-CoV-2 (MOI=0.1) alone or co-infected with eTIP1 particles at different ratios (1:1, 1:10, 1:50) for 1 hour at 37° C. The virus mixture was removed, and cells were further cultured with medium. At indicated time-points, 24, 36, 28 48 hpi (hour post infection), supernatants were collected and viral titers of supernatant were measured with plaque assay on Vero-E6 cells.

Plaque Assay for SARS-CoV-2:

For SARS-CoV-2 plaque assays, 80% Confluent monolayers of Vero E6 cells grown in 6-well plates were incubated with the serial dilutions of virus samples (250 μl/well) at 37° C. for 1 hour. Next, the cells were overlayed with 1% agarose (Invitrogen) prepared with MEM supplemented containing 2% fetal bovine serum. Three days later, cells were fixed with 4% formaldehyde for 2 hours, the overlay was discarded and samples were stained with crystal violet dye.

Mouse Experiments for SARS-CoV-2:

K18-hACE2 mice (The Jackson laboratory, www.jax.org/strain/034860, stock number: 034860, B6.Cg-Tg(K18-ACE2)2Prlmn/J, Hemizygous). The K18-hACE2 mice were inbred and housed in UCSF animal facility. The mice were under anesthesia and at the BSL3 μlevel for all experiments performed in this study. 30 μg eTIP1 RNA with lipofectamine-2000 were inoculated into mice intranasally. 18-20 hours later, K18-hACE2 mice were anesthetized with isoflurane and inoculated with 6×10⁴ PFU of SARS-COV-2 intranasally. The mice were monitored daily and weight were measured at indicated time-points. For tissue distribution, mouse were sacrificed at indication time-points, the tissues were collected and homogenized with 1 mL 2% FBS MEM medium with gentleMACS-C tubes (Miltenyi Biotec Catlog #130-093-237). Plaque assays were performed for titration of the virus. For RNA extraction, the 100 mg tissues were homogenized in 1 mL trizol reagents (Ambion) with gentleMACS-M tube (Miltenyi Biotec, Cat #130-093-236), RNA were treated with DNasel, 1 mg total RNA were used to make cDNA by Iscript™ (Bio-Rad). DNasel treated total 72 RNA, then poly A beads purification (Bio Scientific), then the RNASeq libraries were prepared with the KAPA biosystem (KAPA Stranded RNA-Seq Library Preparation Kit).

Hematoxylin Eosin (H&E), Immunofluorescence(IF) Staining on Tissue Section and Imaging.

For pathology and immuno-fluorescence, mice tissues were collected and fixed in the 4% PFA, then the tissues were embedding with paraffin and wax and processed. The tissue samples were cut at 5 μM, and H&E staining were performed at the Gladstone Histology and light core. Deparaffinization, rehydration, and HIER were performed on an ST4020 small linear stainer (Leica). For deparaffinization, slides were baked at 70° C. for 1-1.5 h, followed by rehydration in descending concentrations of ethanol (100% twice, 95% twice, 80%, 70%, ddH₂0 twice; each step for 30 s). Washes were performed using a Leica ST4020 Linear Stainer (Leica Biosystems, Wetzlar, Germany) programmed to three dips per wash for 30 s each. H& E staining were performed. For I.F., HIER was performed in a Lab Vision™ PT module (Thermo Fisher) using Dako Target Retrieval Solution, pH 9 (S236784-2, DAKO Agilent) at 97° C. for 10 min and cooled down to 65° C. After further cooling to room temperature for 30 min, slides were washed for 10 min three times in Tris-Buffered Saline (TBS), containing 0.1% Tween 20 (Cell Marque; TBST). Sections were then blocked in 5% normal donkey serum in TBST at room temperature for 1 h, followed by incubation with primary antibodies in the blocking solution. After one overnight incubation of primary antibodies in 4° C., sections were washed three times with TBST and stained with the appropriate secondary antibodies in PBS with 3% bovine serum albumin, 0.4% saponin, and 0.02% sodium azide at room temperature for 1 h. Following this, sections were washed three times with TBST and mounted with ProLong Gold Antifade mounting medium with DAPI (Invitrogen). The primary antibodies and final titrations used were mouse anti-acetylated a Tubulin (ACTUB) (1:300; Santa Cruz sc-23950), rabbit anti-SARS-CoV-2 nucleocapsid (N) (1:1000; GeneTex GTX135361), and mouse anti-SARS-CoV-2 spike(S) (1:600; GeneTex GTX632604). Secondary antibodies include highly cross-adsorbed donkey anti-rabbit Alexa Fluor 647 1:500 (Thermo A32795) and highly cross-adsorbed donkey anti-mouse Alexa Fluor 555 1:500 (Thermo A32773). The Immunofluorescence (IF) for poliovirus eTIPI inoculated mice head and lung section was performed using antibody against-poliovirus antibody 3B (Vpg) (1:200). Fluorescence-immunolabeled images were acquired using a Zeiss AxioImager ZI microscope or Keyence BZ-X710 fluorescent microscope. Post-imaging processing was performed using FIJI/ImageJ. The intensity of nucleocapsid (N) and spike (SP) were qualified by mean intensity of the at least ten areas at same places in each tissue section.

Mouse Lung Histological Analysis:

Paraffin-embedded lung tissue blocks for mouse lungs were cut into 5 m sections. Sections were stained with hematoxylin and eosin (H&E) and analyzed. Digital light microscopic scans of whole lung processed in toto were examined by an experienced veterinary pathologist. Hematoxylin Eosin (H&E) stained sections of lung from K18 hACE2 mice were examined by implementing a semi quantitative, 5 point grading scheme (0—within normal limits, 1-mild, 2-moderate, 3-marked, 4-severe) that took into account four different histopathological parameters: (1) perivascular inflammation; (2) bronchial or bronchiolar epithelial degeneration or necrosis; (3) bronchial or bronchiolar inflammation; and (4) alveolar inflammation. These changes were absent (grade 0) in lungs from vehicle and Plitidepsin treated uninfected mice from groups that were utilized for this assessment.

### Example 13. mRNAseq Libraries Preparation and Analysis

Mice tissue were collected and homogenized in 1 ml Trizol reagents (Ambion). Total RNAs were extracted, 1 mg treated with DNase 1 (NEB). Then the mRNAs were purified by polyA beads, mRNAseq libraries were prepared by followed the instruction with KAPA Biosciences. Then mRNAseq libraries were pooled and sequenced by Illumina HiSeq 4000 with single read in the UCSF core facility (Center for Advanced Technology, http://cat.ucsf.edu).

Differential gene and transcript expression analysis of mRNA-seq experiments with TopHat-Cufflinks-Cuffdiff pipeline. The Figures were plot by R, CommundR and ggplot2 packages.

### Example 14. Data Analysis and Statistical Analysis

Data were presented as mean±SD. Statistical analysis was performed using GraphPad Prism (GraphPad Software). Statistical significance was calculated using a two-tailed Student's t-test and p-value<0.05 was considered significant. Significance is noted with asterisks as described in the figure legends. Animal experiments were not blinded or randomized, and no animals or samples were removed as outliers from the analysis. The 50% lethal dose (LD50) and Survival curves were compared with Log-rank (Mantel-Cox) test methods performed using GraphPad Prism (GraphPad Software). In these experiments, p-value<0.05 was considered significant.

### Example 15. Engineering a Defective Poliovirus Genome as a Broad-Spectrum Antiviral

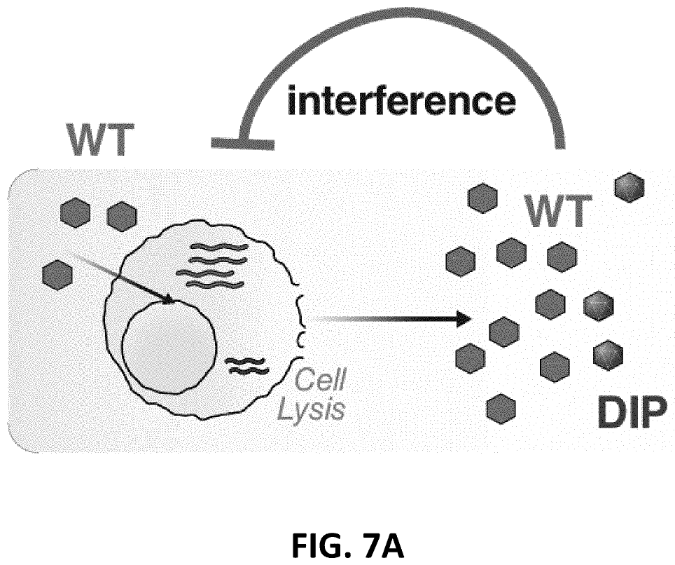

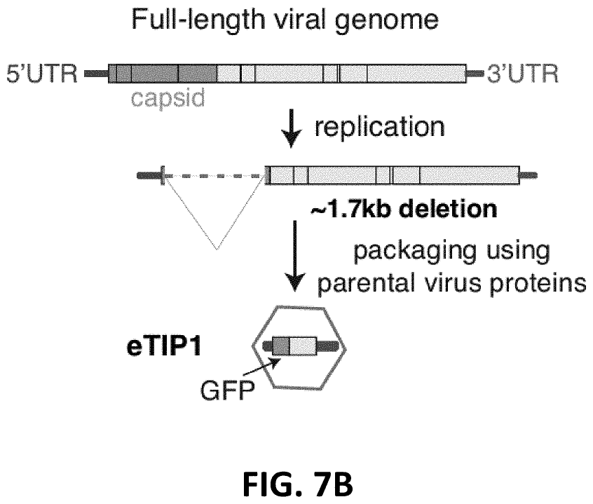

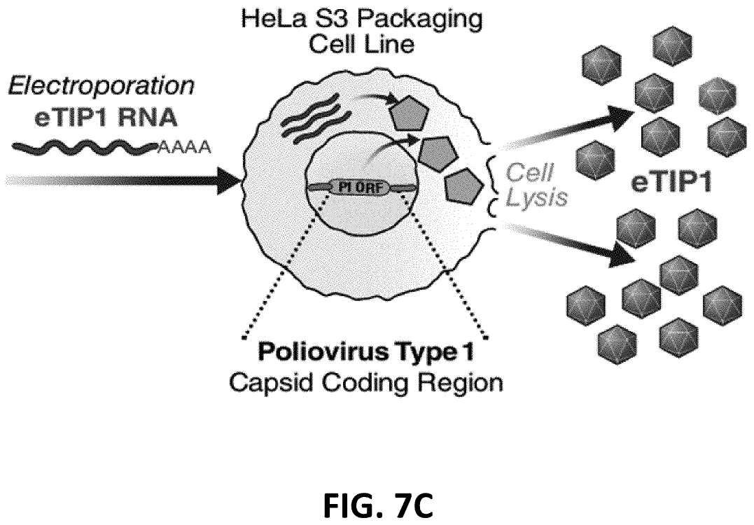

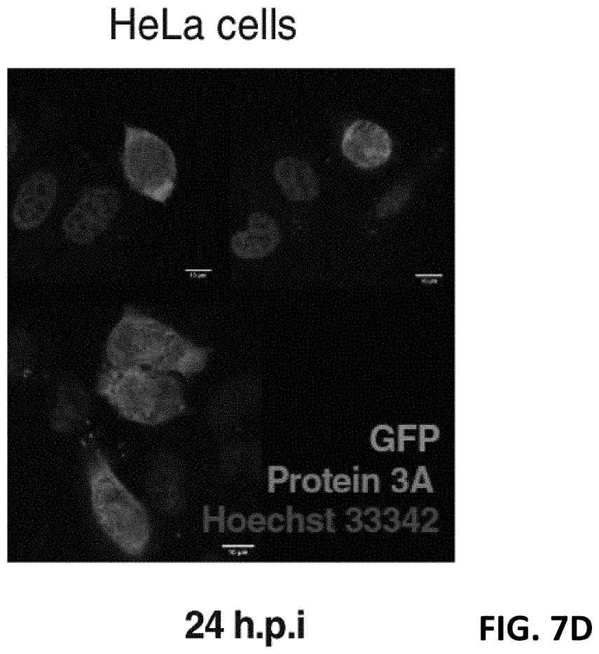

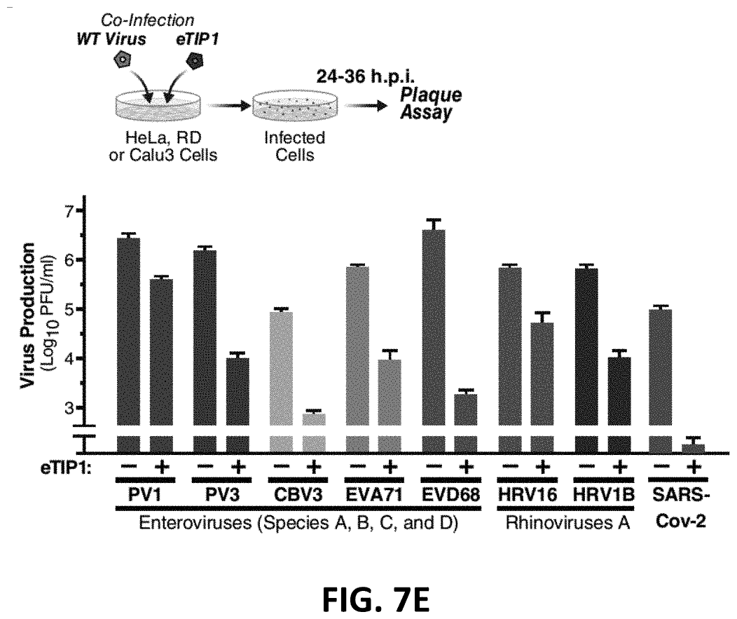

This Example describes experiments performed to demonstrate the concept that DIPs as disclosed herein can inhibit enterovirus replication (FIG. 7A). In these experiments, a well-studied poliovirus was used as the model.

A DVG (defective viral genome) for poliovirus type 1 (PV1) (herein eTIP1; i.e., enteroviral therapeutic interfering particle 1) was engineered by deleting the entire P1 region encoding a structural proteins and inserting a GFP gene (see, e.g., FIG. 1A and FIG. 7B). eTIP1 infectious particles were produced using a packaging cell line that stably expresses the PV1 capsid protein precursor P1 (Hela S3/P1) (see, e.g., FIG. 1B and FIG. 7C). This packaging cell line facilitates the generation of eTIP1 particles by transfecting the in vitro transcribed RNA of eTIP into the packaging cells. Transfection of HeLaS3/P1 with in vitro transcribed eTIP1 RNA generated eTIP1 infectious particles, which were amplified by repeated infection of HelaS3/P1. High-titer (>10⁸ infectious units/ml) were purified by sucrose cushion and gradients to 95% purity as determined by silver stain-polyacrylamide gel electrophoresis and negative staining electron microscopy (EM). (see, e.g., FIG. 1C; see also Example 8). Purified eTIP1 particles could infect cells, and this was determined by expression of GFP and immunofluorescence (I.F.) with polio-3A antibody staining (see, e.g., FIG. 7E). eTIPI did not spread from cell to cell without a wildtype (WT) PV1 acting as a helper. A panel of wild-type viruses and eTIP in cell culture at ratio of 1:20 was co-infected. eTIP inhibited all of the enteroviruses tested including type 1 and type 3 poliovirus (PV1 and PV3), coxsackie virus B3 (CVB3) and enterovirus D68 (EV-D68), Rhinovirus 1A and 1B (see, e.g., FIGS. 1A-1E and FIGS. 7A-7E). The virus replication was reduced 10-100 fold in co-infection on Hela S3 cells; the virus replication was measured by plaque forming assay or TCID50 on cells (FIGS. 1D and 7D; see also Example 3). Interestingly, eTIP inhibited viral replication greater in EV-D68 and the Rhinovirus group than the PV1, PV3 and CVB3 by coinfection. The cell culture experiments suggest that the eTIP could inhibit virus replication and production by co-infection and that eTIP inhibits EV-D68 stronger than other viruses suggesting that the level of inhibition is related to the dynamics of the virus replication.

### Example 16. eTIP Confers Broad-Spectrum Antiviral Activity in a Mouse Model

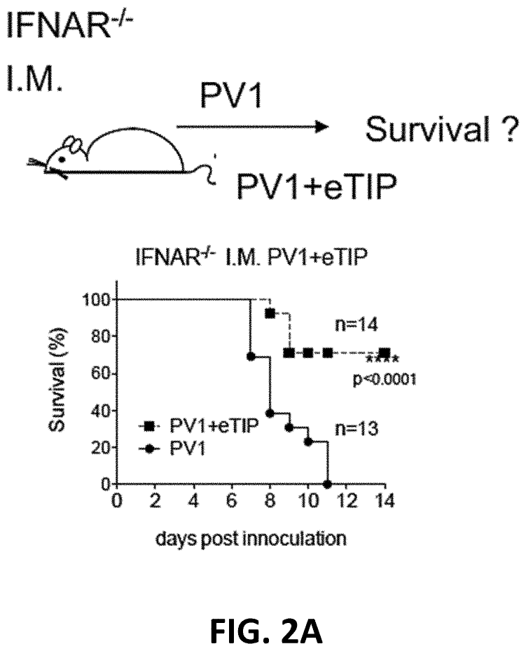

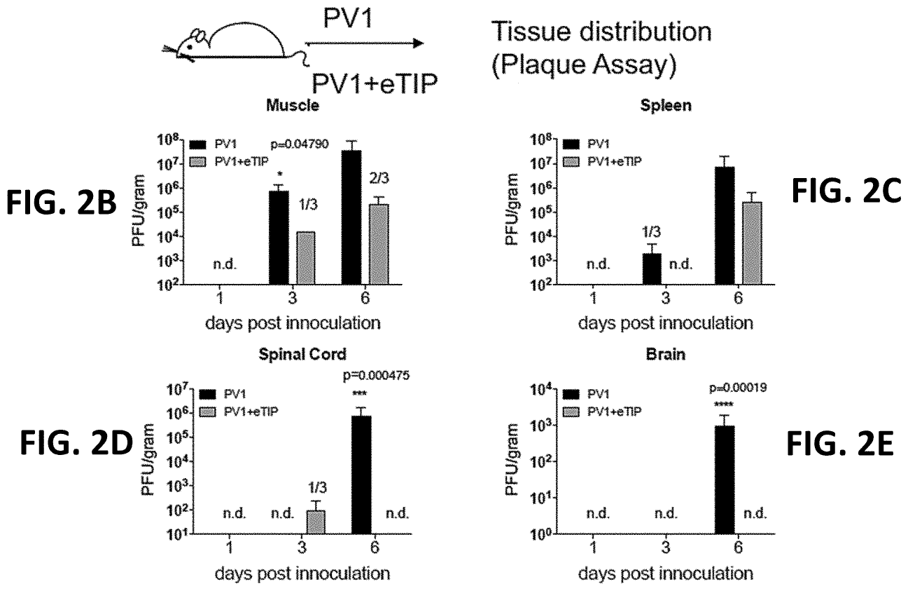

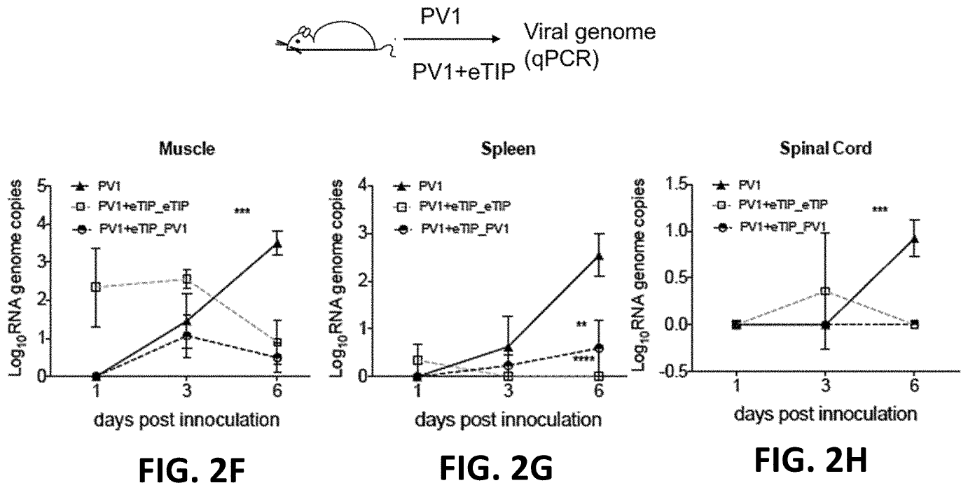

This Examples describes experiments performed to evaluate the protective effect of eTIP in infected animals by measuring the survival rate. In these experiments, immune compromised mice, Tg21 PVR interferon α/β receptor knockout (IFNAR^{-/-}) mice were infected with 200 plaque forming units (PFU) wild-type poliovirus type 1 (PV1) by intramuscular (I.M.) route, which is the 5 times as 50% lethal dose. IFNAR^{-/-} mice were infected with PV1 alone or co-infected with mixed PV1+eTIP at a ratio of 1:5000 by single time point infection (Methods). 100% of IFNAR^{-/-} mice infected with PV1 alone were killed within 14 days post-infection (n=13-14). In contrast, 70% of mice co-infected with PV1+eTIP, survived 14 days post-infection (11 of 14, p<0.001). These data showed that eTIP significantly increases the survival rate (Log-rank (Mantel-Cox) test). The protection rate was ˜70% with a single treatment (see, e.g., FIG. 2 and Table 2).

TABLE 2
Co-infection DIP protection in IFNAR^{-/-}
IFNAR^{-/-}

| | Mice | Virus | Infection | WT/DIP | | | Protection | |
|---|---|---|---|---|---|---|---|---|
| | Strain | Strains | route | ratio | WT dose | | rate | Dose |
| 1 | IFNAR−/− | PV1 | I.M. | 1:250 | 200 | PFU | 70% | Single |
| 2 | IFNAR−/− | PV1 | I.P. | 1:10 | 10⁴ | PFU | 0% | Single |
| 3 | IFNAR−/− | PV1 | I.M. | 1:100 | 200 | PFU | 60% | Single |
| 4 | IFNAR−/− | PV1 | I.M. | 1:10 | 200 | PFU | 20% | Single |
| 5 | IFNAR−/− | PV3 | I.M. | 1:250 | 1000 | PFU | 30% | Single |
| 6 | IFNAR−/− | CVB3 | I.M. | 1:15000 | 100 | PFU | 40% | Single |
| 7 | IFNAR−/− | CVB3 | I.M. | 1:2500 | 20 | PFU | 20% | Single |
| 8 | IFNAR−/− | EV-D68 | I.C. | 1:1000 | 2 × 10³ | | 40% | Single |
| | | | | | TCID50 | | | |
| 9 | IFNAR−/− | CVB3 | I.M. | 1:250 | 200 | PFU | 1 day | Single |
| | | | | | | | delayed | |
| | | | | | | | death | |
| 10 | IFNAR−/− | PV1 | I.M. | 1:7500 | 200 | PFU | 80% | Multiple- |
| | | | | | | | | 5 days |
| 11 | IFNAR−/− | PV3 | I.M. | 1:1000 | 10000 | PFU | 50% | Multiple- |
| | | | | | | | | 5 days |
| Therapeutics | IFNAR−/− | PV1 | I.C. DIP, | 1:7500 | 200 | PFU | 1 day | Single |
| | | | IM. PV1 | | | | delayed | |
| | | | | | | | death | |

### Example 17. eTIP Inhibits Wild-Ty e Virus Spread from Muscle to Central Nervous System (CNS) of Infected Animals

This Example describes experiments performed to investigate whether eTIP inhibits PV1 spread in infected animals. IFNAR^{-/-} mice were infected with 200 PFU PV1 virus alone or co-infected with PV1+eTIP at a ratio of 1:5000 by I.M. route (Methods and FIG. 2). Muscle, spleen, spinal cord, brain was collected at indicated time points [Methods]. In the PV1 group, PV1 viruses replicated rapidly in muscle and spread to spinal cord and brain; virus titer was measured by plaque assay (see, e.g., FIGS. 21-2E). In contrast, in the PV1+eTIP co-infection group, the level of PV1 viruses was 100-fold lower in the muscle and spleen at 3 and 6 days-post-infection (see, e.g., FIGS. 21-2E) and PV1 was undetectable in the spinal cord and brain at 6 days-post-infection (see, e.g., FIGS. 2B-2E n=3, p<0.01). These data suggests that the PV1 virus spread is largely inhibited by eTIP even with a single treatment.

RNA levels of PVI virus and eTTP from the tissues were also measured, because RNA genome copy is much more sensitive than virus titer. The viral RNA genome copy per 1 μg total RNA was measured by digital droplet RT-qPCR [Methods]. Consistent with the virus titer results (see, e.g., FIGS. 21B-2E), PV1 viral RNA levels increased rapidly in muscle and spleen after infection by PV1 virus alone. In contrast, in the co-infected group, the RNA genome copies number of PV1 was 100-fold lower in muscle and 1000 times lower in spleen compared to the PV1 group. Furthermore, the RNA genome of PV1 in co-infected group was undetectable in the spinal cord (see, e.g., FIGS. 2F-2H). These data suggest that eTIP inhibits wild-type virus PV1 spread to the central nervous system (CNS) of infected animals and increases the survival rate of the PV1 infected animals.

### Example 18. Evaluating the Protective Effect of eTIP at Different Ratios

This Example describes experiments performed to test how the ratios of PV1 and eTIP affect the protective effect. In these experiments, IFNAR^{-/-} mice were infected with 200 PFU PV1 alone or co-infected with different ratios of eTIP (PV1+eTIP, ratio=1:10, 1:100) by intra-muscular infection. 100% of IFNAR^{-/-} mice infected with PV1 alone, were dead 14 days post-infection (n=8-10). In contrast, co-infection with PV1+eTIP at a ratio of 1:100 significantly increased the survival rate. The protection rate was 60% with a single treatment (Log-rank Mantel-Cox test, n=8-10, p<0.05). Mice co-infected with PV1+eTIP at a ratio of 1:10 did not differ significantly different compare to PV1 alone; 80% (8 of 10) mice were dead 14 days post-infection (FIG. 3A). These data suggests that the protection was most likely due to direct competition because the protection rate of eTIP correlates with the ratio between PV1+eTIP at 1:10 and 1:100 (see, e.g., FIG. 3A and Table 2). The ratio greater than 1:100 such as 1:250(data not shown here), the protection rate remains around 70%.

Subsequent experiments were performed to test whether eTIP can protect mice challenged with a lethal dose infection of other enterovirus strains and indeed, eTIP conferred protection from all virus strains tested. For the type 3 poliovirus (PV3) group, the protection rate was 30% (n=20, p=0.0337) with PV3+eTIP at a ratio 1:250 (see, e.g., FIG. 3B). For the Coxsackievirus B3 strains (CVB3) group, the protection rate was 20% (n=20, p=0.0221) with CVB3+eTIP at a ratio of 1:5000 (see, e.g., FIG. 3C). The protection rate was 40% with EVD68+eTIP at a ratio of 1:1000 by I.C. route (n=8-10, P<0.001) (see, e.g., FIG. 3D). These data suggest that the eTIP has the potential to protect against polioviruses and non-polio enteroviruses in infected animals. With being bound to any particular theory, the observed effect could be attributed to direct competition by local infection in IFNAR^{-/-} mice because the protection rate correlates with the similarity of the genomic sequencing and the initial ratio which was delivered into immune compromised animals. The safety of eTIP was also tested by UV treating the eTIP and introducing to Tg21PVR, IFNAR^{-/-} mice by I.M. infection. It was observed that none of these mice died, suggesting that eTIP itself is safe in animals (see, e.g., FIGS. 3E-3F).

### Example 19. The Protection Effect of eTIP in Immune Competent Mice and the Role of IFN Responses

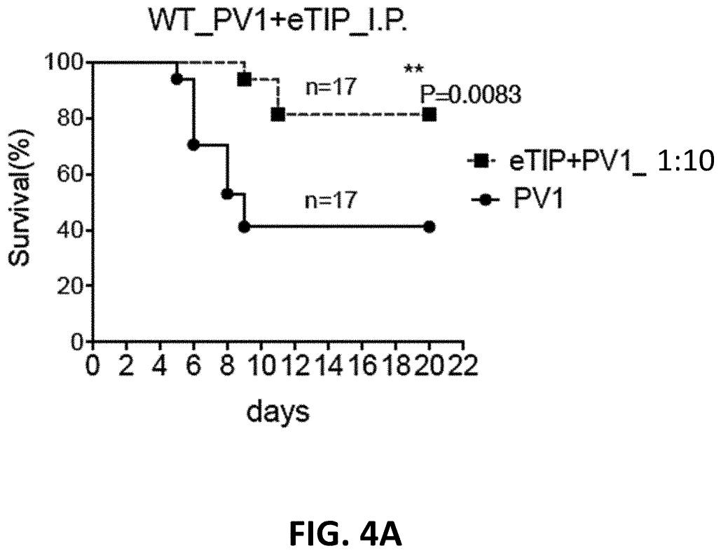

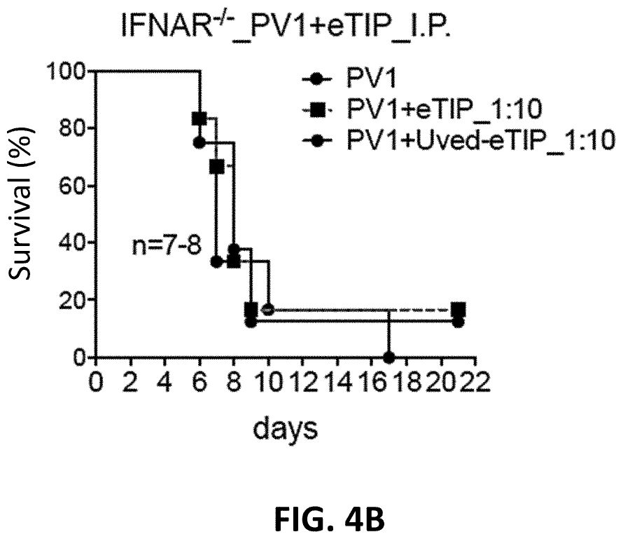

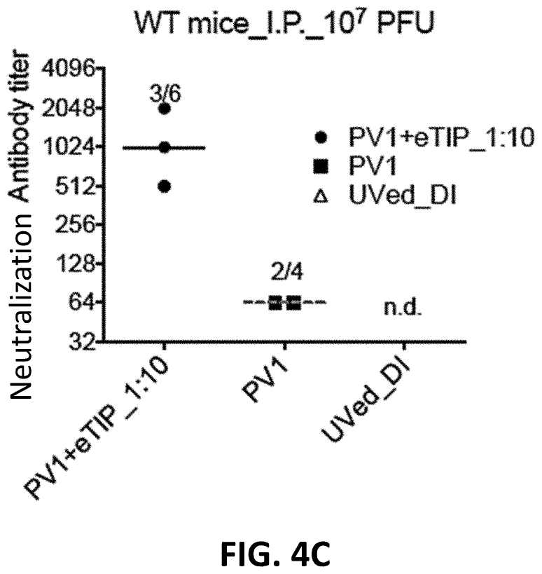

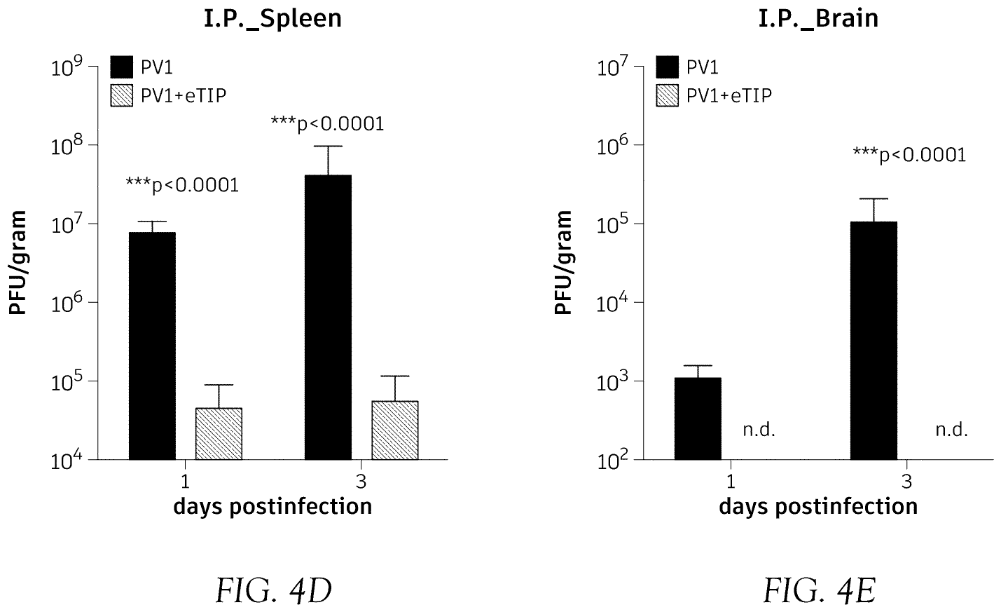

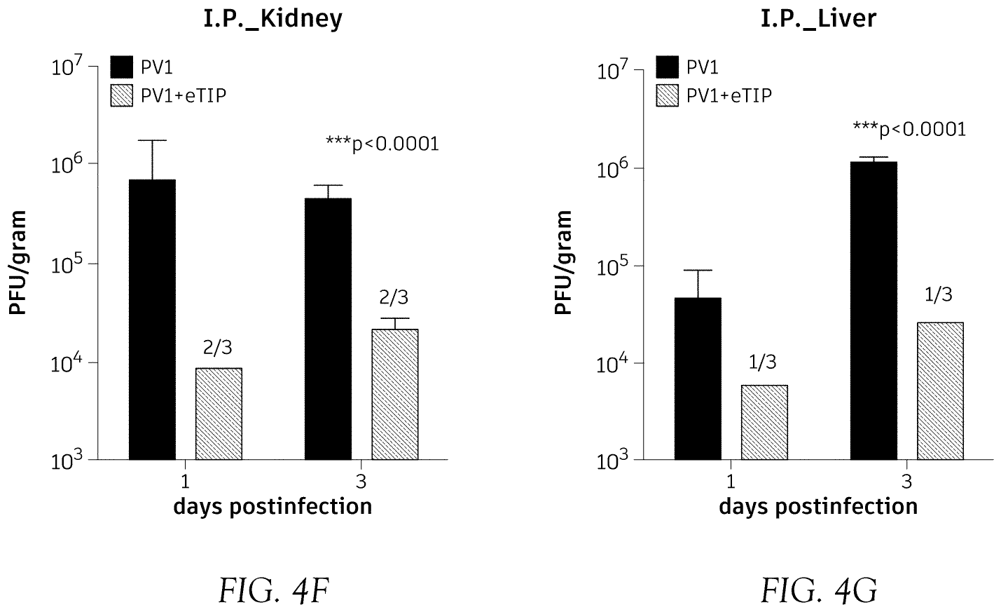

This Example described several additional control experiments conducted to further examine whether IFN responses play important roles on the protection in immune competent mice. These experiments are important to understand the mechanism of protection, which can illuminate ways to produce an even more effective therapy. For example, it is possible that cytokine produced by packaging cell lines are present in the eTIP preparation, and that protection is mediated by these contaminants. To address this important question, the eTIP were purified to 95% purity as determined by silver stain polyacrylamide gel electrophoresis and EM negative stain (see, e.g., FIG. 1C). Immune competent mice Tg21PVR or IFNAR^{-/-} were infected with PV1 virus alone (10⁷ PFU or 10⁴ PFU respectively, ˜10 times lethal dose 50%) or co-infected with eTIP at ratio 1:10 by I.P. alone (see, e.g., FIGS. 4A-4B and Table 3).

TABLE 3
Wild-type Tg21PVR mice

| | | | Infection | WT/DIP | | | | |
|---|---|---|---|---|---|---|---|---|
| | Mice Strain | Virus | route | ratio | WT dose | | Protection rate | Dose |
| 1 | Wildtype Tg21 | PV1 | I.P. | 1:10 | 10⁷ | PFU | 40% protection | Single |
| 2 | Wildtype Tg21PVR | PV1 | I.N. | 1:30 | 2 × 10⁵ | PFU | 60% protection | Single |

TABLE 4
Therapeutic

PV2 2 × 10⁵ PF, I.N.

| +DIP-IN-day 3-4 | +DIP-IN-day 1-5 | No-DIP |
|---|---|---|
| 20% survival 1/5 | 65% survival 11/17 | 19% survival 4/21 |

TABLE 5
Prophylactic

PV2 2 × 10⁵ PF, I.N.

| −24 h DIP-IN | −48 h DIP-IN | No-DIP |
|---|---|---|
| 20% survival 1/5 | 87.5% survival 14/16 | 20% survival 4/21 |

In Tg21PVR mice, co-infection group (PV1+eTIP=1:10), eTIP increased survival rate by 40% compared to PV1 group alone at 21 days post-infection. In contrast, in the IFNAR^{-/-} mice groups with same ratio (PV1+eTIP=1:10), eTIP did not show any protection against PV1, as the survival rate was same in all IFNAR^{-/-} groups (see, e.g., FIG. 4B). The ratio of the virus to eTIP of 1:10 was used because this is the highest amount of eTIP that was possible after the concentration and purification.

Subsequent experiments were then performed to investigate whether the surviving mice generated adaptive immunity. Sera were collected from the mice at 28 days post-infection (see, e.g., FIG. 4A) and neutralization antibody (NAb) titrations were tested on cell culture. In the PV1+eTIP group, the level of NAb was significantly higher than compared to the PV1 group (512-fold dilution compared to 64-fold dilution) (see, e.g., FIG. 4C). NAbs were not detected in the UV-inactivated eTIP group (see, e.g., FIG. 4C).

It was observed that a high ratio of eTIPs was required for protection in interferon-defective IFNAR^{-/-} animals (FIGS. 2 and 3), while in wild-type mice a lower ratio was sufficient (FIG. 4). This indicates the possibility that the host immune response play important roles for eTIP protection. Interestingly, UV-inactivated eTIP did not show any protection effect in Tg21PVR and IFNAR^{-/-} groups, suggesting that the replication of eTIP is required for protection. Furthermore, the data presented herein suggest the eTIP probably stimulates the host adaptive immunity and may be used as an adjuvant for vaccine production because the co-infected eTIP with wild-type virus (PV1+eTIP) increases NAb generation and the adaptive immunity.

Subsequent experiments were then performed to further investigate the virus spread and replication in different tissues. Tg21 PVR mice were infected with the PV1 or PV1+eTIP at a ratio of 1:10 and spleen, kidney, liver and brain were collected at the indicated time points (see, e.g., FIGS. 4C-4E and Methods). From all the tissues tested, the coinfection group (PV1+eTIP) had significantly reduced the virus replication and the viral loads were reduced at least 2 μlogs (see, e.g., FIGS. 4C-4E). By systematic infection with I.P. route, the protection effect perhaps due to induce different patterns of immune responses by co-infecting with PV1 and eTIP, compared with wild-type virus alone.

### Example 20. eTIP Inhibits Poliovirus in Primary Cells and Induces Interferon Response by Co-Infection

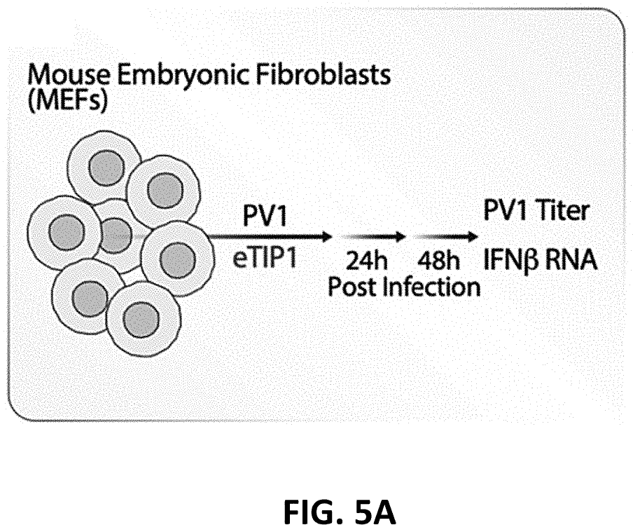

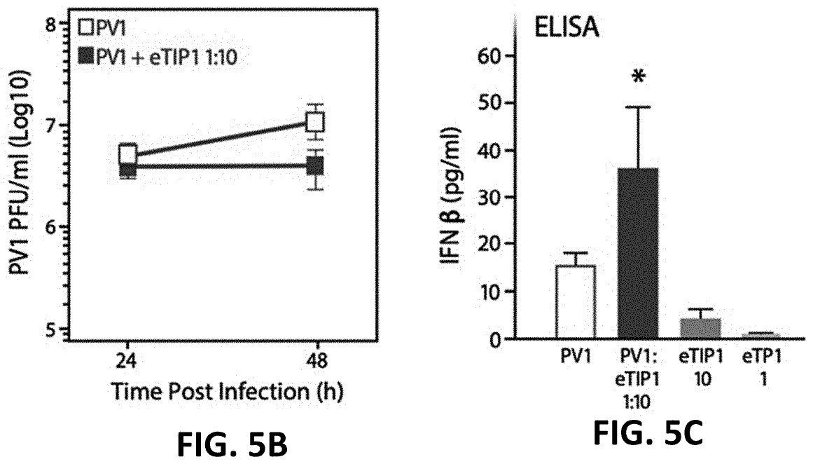

This Example describes experiments performed to evaluate the eTIP protection effect in primary murine (MEFs derived from Tg21PVR mice. MEFs were infected with PV1 alone, eTIP alone, or co-infected with different ratios of PV1 with eTIP. The replication of PV1, eTIP, and the induction of interferon were then measured. In PV1 infection group, the virus production was increased about 10-fold, while in the group co-infected with eTIP at a ratio of 1:10, the wild-type production of PV1 was 10-fold lower compared to the control. Interestingly, the IFN induction at 48 hours increased about 3-fold higher than PV1 or eTIP alone as measured by ELISA. The virus production was measured by plaque assay and the eTIP level was measured by the GFP positive cells in the Hela S3 cells. These data suggest that (i) the Co-infected eTIP with PV1 at a ratio of 1:10 increases the interferon induction; (ii) the induction of interferon requires the virus replication; and (iii) the ratio between PV1 and eTIP is an important factor on the inhibition effect on PV1 and the interferon induction. These data are consistent with the observation in wild-type PVR mice (see, e.g., FIG. 5).

### Example 21. eTIP Confers Prophylactic and Therapeutic Antiviral Activity in the Respiratory Tract Infection and Mucosal Immunity Plays an Important Role on eTIP Protection

This Example describes experiments performed to illustrate that eTIP confers prophylactic and therapeutic antiviral activity in the respiratory tract infection and mucosal immunity plays an important role on eTIP protection. It has been reported that poliovirus can infect immune competent mice Tg21PVR mice intranasally. This is important because poliovirus replicates in the upper respiratory tract and invades the CNS very rapidly through the olfactory nerve, causing severe disease. The experiments described in this Example were designed and performed based on the hypothesis that the eTIP can inhibit PV1 replication by co-infected poliovirus with intranasal inoculation. In these experiments, immune competent mice Tg21 PVR strain (Tg21) were infected with 2×10⁵ PFU PV1 virus (˜5 times as 50% lethal dose) alone or co-infected with mixed PV1+eTIP at ratio 1:30 by intranasal (I.N.) route. 80% of Tg21PVR mice infected with PV1 virus alone were dead (n=17) 21 days post-infection. Strikingly, in the co-infection group, 80% mice survived up to 21 days (n=13, p<0.001). In contrast, the same ratio of eTIP that protected PV1 in wild-type mice, did not protect in IFNAR^{-/-} mice (see, e.g., FIG. 6). These data suggest that at a lower ratio (1:30), the eTIP protection effect is higher in immune competent mice by intranasal (I.N.) infection. Further, the data indicate the protection effect requires the eTIP to replicate because the UVed eTIP did not show any of the protection in both wild-type and IFNAR^{-/-} mice.

To test the therapeutic effect of eTIP, Tg21 PVR strain (Tg21) mice were infected intranasally with 2×10⁵ PFU PV1 virus (˜5 times as 50% lethal dose)18, and after 24 hours eTIP (6×10⁶ IU/mouse) was delivered, and this process was repeated daily for total five days. 80% of mice in PV1 group (n=21) were dead after 21 days, while in the therapeutic group with eTIP, the death rate was 30% (n=17, p=0.0039). The protection rate of eTIP was approximately 50%. Interestingly, delivery of eTIP only at Day 3-Day 4 post-infection did not produce a protective effect for the therapeutic experiments. In addition, the prophylactic effect of eTIP was also tested. In these experiments, Tg21PVR mice were inoculated with eTIP 24 hours or 48 hours prior to PV1 infection. No protection effect was observed for the 24 hours prophylactic group (n=5), while eTIP conferred 60% protection for the 48 hour group. 80% of mice survived by I.N. infection, compared with the PV1 group in which 20% mice survived.

Additional experiments were carried out to further test if eTIP can protect against other respiratory tract RNA viruses. Influenza H1N1 was co-infected with eTIP in mice by intranasal inoculation. Indeed, the eTIP reduced the weight loss compared with influenza A H1N1 PR8 strain alone. Without being bound to any particular theory, it is believed that eTIP can induce the host interferon response, especially induce the mucosal immunity in the respiratory tract. It is further contemplated that cell-type specific immune responses such as dendritic cells, it is possible that macrophages or T cells responses were induced in the context of these conditions.

### Example 22. eTIP1 DVG Therapeutic Potential

This Example describes experiments performed to investigate therapeutic potentials of eTIP DVG described herein. First, whether eTIPI can block replication of PV1 and a set of related enteroviruses of clinical importance was determined. The cells were infected with enteroviruses in the presence or absence of eTIP1 at a ratio of 1:20 (eTIP1 multiplicity of infection (M.O.I.)=1 to 5. eTIP1 effectively blocked the replication of PV and other enteroviruses, including circulating pathogenic enterovirus EV-D68, EV-A71, coxsackievirus (CVB3) and rhinoviruses (see, e.g., FIG. 7E; see also Example 1). Virus replication was inhibited 10-1000-fold, depending on the virus and the cell line examined (see, e.g., FIG. 7E). To determine whether eTIP1 antiviral activity was restricted to its viral genus, eTIP1 activity against an unrelated virus was tested. Lung epithelial Calu-3 cells were infected with SARS-CoV-2 alone or with eTIP1 at different ratios, SARS-CoV-2 were reduced at least 100 folds at the ratio 1:50 (FIG. 7E). The protection effect was dose dependent, as observed when the ratio was increased from 1:10 to 1:50 on EV-D68 (see, e.g., FIG. 1G).

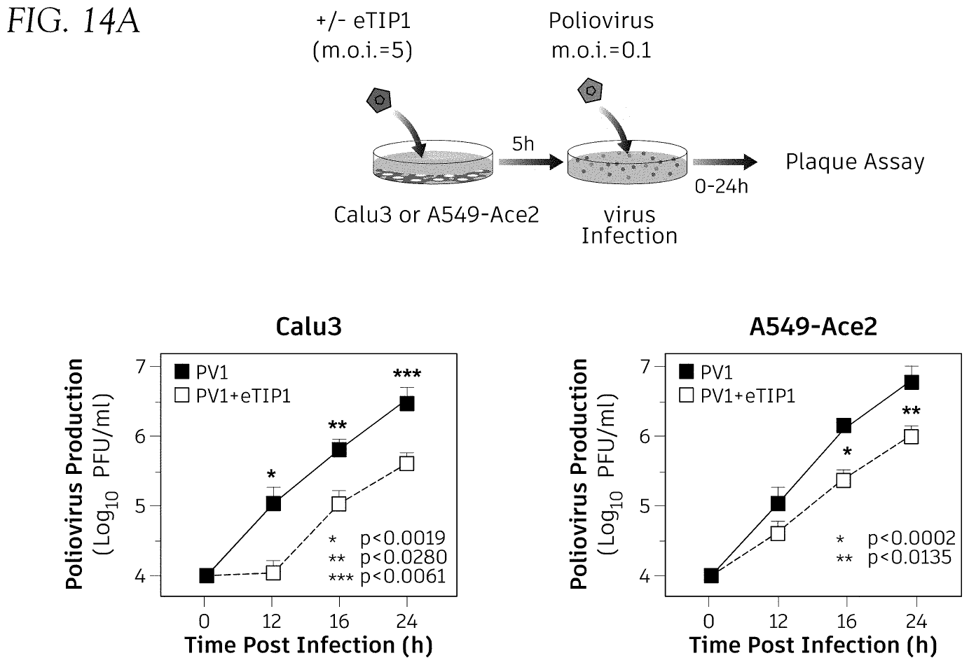

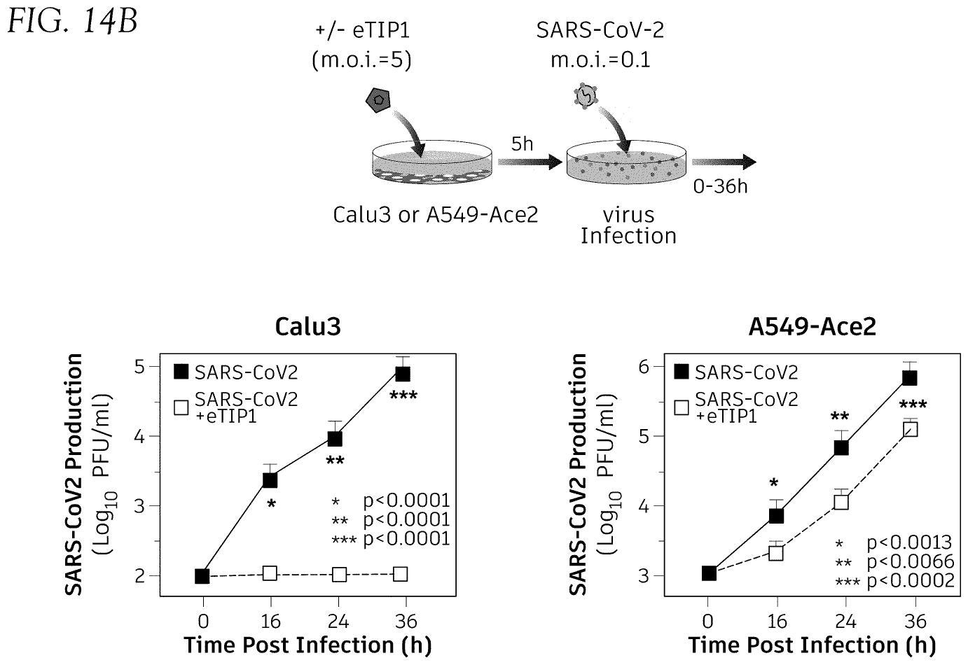

To further examine the eTIPI interference process, growth curves was tested. Lung epithelial Calu-3 or A549-Ace2 cell lines were infected with eTIP1 (M.O.I.=5), and 5 h later, the cells were infected with poliovirus or SARS-CoV-2. Virus titers were determined at several times post-infection. Strikingly, eTIP1 was found to also inhibit replication of poliovirus or SARS-CoV-2 prophylactically (see, e.g., FIGS. 14A-14B). These data suggest that the inhibitory activity of eTIP1 does not rely exclusively on direct competition for enteroviral proteins, such as the viral capsid or other enteroviral factors required for replication.

### Example 23. eTIP1 Prevents Lethal Infection in Mice

This Example describes experiments performed to examine eTIPI's ability to prevent lethal disease in mice infected with a highly pathogenic poliovirus type 1 Mahoney strain (PV1), given the surprisingly broad-spectrum inhibitory effects of eTIPI in cell culture. After intraperitoneal inoculation (IP) in PV1-susceptible immune-competent transgenic mice (Tg21), PV1 replicates and accumulates at high titers in diverse tissues, ultimately reaching the central nervous system (CNS) and causing paralysis and death. Similarly, PV1 infection of the upper respiratory tract (IN) reaches the CNS rapidly through the olfactory nerve, causing infection of the spinal cord and brain to develop severe disease.

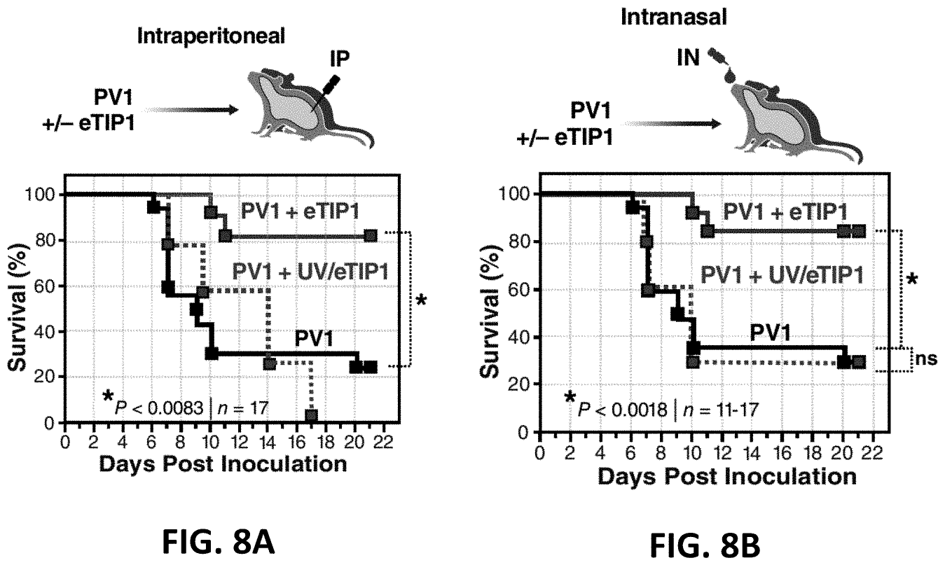

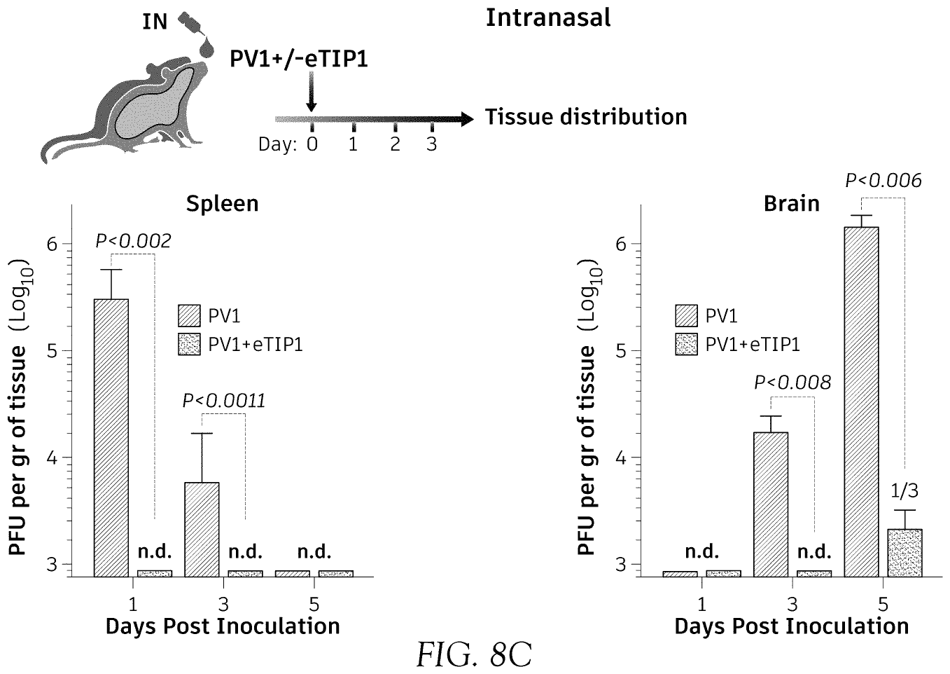

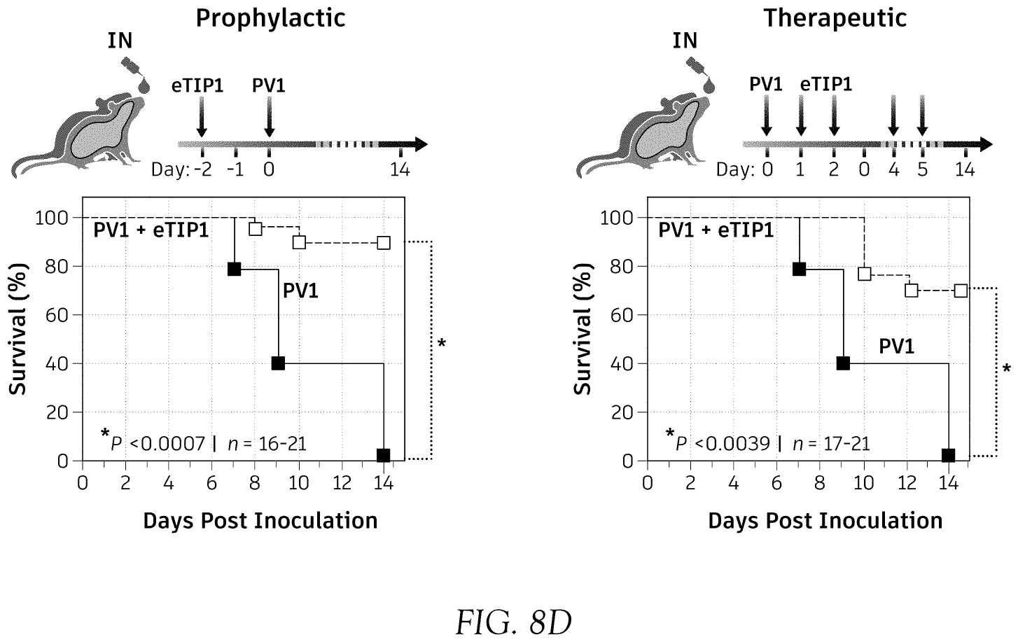

Thus, mice were infected by the intraperitoneal (FIG. 8A) or intranasal (FIG. 8B) routes with high doses of PV1 (10⁷ PFU or 3×10⁵ PFU, respectively) with or without co-infection with eTIP1. Under these conditions, eTIP1 significantly attenuated disease and protected 80 to 90% of the mice from lethal infection. Importantly, co-inoculation of eTIP1 inactivated by ultraviolet irradiation did not protect the mice from PV1, indicating that the eTIP1 genomes must retain their self-amplification ability for antiviral activity to occur. This study also ruled out non-specific protection by an unknown or undetected contaminant accumulated during eTIP1 production. Of note, eTIP1 protected animals from death after co-infection with several other enterovirus, including EV-D68 (data not shown). To examine the effect of eTIP1 virus replication on tissues, the spleen and brain of animals infected intranasally with either PV1 or PV1+eTIP1 were collected at days 1, 3 and 5. Virus titers were determined by plaque assay. eTIP1 caused a dramatic reduction in PV1 viral loads, ranging from over 500-fold to undetectable levels (FIG. 8C). These results indicate that eTIP1 can protect animals from death caused by highly pathogenic viruses even after a single-dose intranasal administration.

Next whether eTIP1 can maintain its antiviral activity over time was examined. A single dose of eTIP1 was administered intranasally, and animals were challenged 48 h later with pathogenic PV1. Remarkably, while animals inoculated with PV alone succumbed to infection, pre-treatment with eTIP1 protected 90% of the animals from lethal disease (FIG. 8D, left). In addition, treatment with eTIP1 24 h after PV1 infection also elicited significant protection (FIG. 8D, right). The protective effects of eTIP1 inoculation were lost after 72 h (not shown). These results indicate that eTIP1 has both prophylactic and therapeutic protective activity against intranasal infection, preventing severe disease and death.

### Example 24. The Role of Interferon (IFN) in eTIPI-Mediated Antiviral Protection

This Example describes the results of experiments performed to investigate the mechanism by which eTIP1 induces a systemic antiviral protective effect. In principle, two models can be considered. One posits that DVGs outcompete the full-length viral genome for cellular resources and encapsidation by structural proteins, impairing propagation of parental virus to other cells in the tissue. The smaller genome of DVGs may provide a replication advantage over the full-length genome. An alternative possibility is that DVG viral proteins or functions required to disable the host antiviral responses are still sensed by the innate immune machinery. RNA viruses have evolved multiple functions that inactivate different components of host innate immune response. Self-replicating RNAs, particularly DVGs forming cytosolic dsRNA intermediates, activate pattern recognition receptors and trigger innate immune responses that lead to production of IFN-stimulated genes (ISGs). In this way, DVGs may induce a systemic antiviral state that interferes with replication of WT virus. In addition, DVGs may cause cells to lose the integrity of their plasma membrane and release damage-associated molecular patterns that recruit various types of circulating leukocytes to the site. While the first option requires co-infection of DVG and WT virus for interference to occur, the second model is consistent with DVGs impairing viral replication in a non-cell autonomous manner.

The data provided herein are consistent with the second mechanism, in which eTIP1 blocks viral replication by inducing an antiviral state within the respiratory tract. To test this hypothesis, unbiased transcriptome profiling analyses was performed on lung and spleen samples of eTIP1- or mock (PBS)-treated mice. RNASeq analyses of lung at day 2 post-intranasal administration of 6×10⁶ infectious units (IUs) of eTIP1 identified genes altered in expression by greater than four folds in response to eTIPI administration, compared to mock-infected mice (with a false discovery rate, q<0.05, N=4014) (see, e.g., FIG. 9A). This analysis showed eTIP1 induced a set of known immune genes and bona fide anti-viral genes. These genes showed striking similarity in their transcriptional responses (see, e.g., FIG. 9A, heatmap), emphasizing their tight co-regulation during eTIP1 infection. A cluster of highly upregulated genes consisted of type I interferon (IFN)-responsive genes, including WIT 2, IFIT3, WIT 3b, IFITM3, IRF7, ISG15, Mx1, Mx2, STAT1, and a number of OAS paralogs.

Next, flow cytometry analysis was carried out on immune cells of lung tissue after intranasal infection with 6×10⁶ IU of eTIP1. These experiments revealed that eTIP1 infection induces the recruitment of specific lymphoid cells into the lung, including eosinophils (EOS) and plasmacytoid dendritic cells (pDCs) (see, e.g., FIG. 9B), consistent with the induction of innate immune responses.

The above experiments support the notion that eTIP1 induces an IFN-mediated antiviral state. Next, whether eTIP1-mediated antiviral protection requires systemic innate immune stimulation of an IFN response was tested. To this end, the therapeutic capacity of eTIP1 to protect animals lacking the IFN-α/α receptor (IFNAR^{-/-}) against PV1 infection was investigated. Strikingly, under conditions in which eTIP1 elicits a robust protective activity in immunocompetent WT mice (see, e.g., FIGS. 2A-2D), the antiviral protection was completely lost in IFNAR^{-/-} mice (see, e.g., FIGS. 9C and 9D). This indicates that IFN responses are important for the eTIP1-mediated protection from intranasal infection with pathogenic PV1.

Based on these findings, following model for DVG viral interference by eTIP1 is proposed herein. eTIP1 replicates in the initial infected cells at the site of administration, without spreading to other cells (see, e.g., FIG. 9E). Its dsRNA replication intermediates are recognized by pattern recognition receptors, leading to synthesis of type I IFN and inducing potent innate responses. Because the PV protease and other non-structural viral proteins in the eTIP1 induces profound rearrangements of cellular organelles and pathways, eTIPI can also lead to cellular necrosis and leakage of cytoplasmic fluid. This will recruit leukocytes into the tissue and promote an immuno-competent environment. Thus, molecular processes triggered by the eTIP1 mimic the events of a “natural infection” that recruit different arms of immune defense system in a balanced manner, generating a systemic antiviral response. Of note, mucosal challenges with RNA viruses achieve sterilizing immunity with no adverse effects involving short- or long-term immune dysfunction. Data provided herein suggest that modulation of natural innate antiviral immunity is a primary determinant of the eTIP1 antiviral activity that inhibits virus replication. Importantly, it was found that a single intranasal dose prevents progression to severe viral disease for a period of time longer than a single-dose administration of a small-molecule antiviral or therapeutic monoclonal antibodies.

### Example 25. Induction of an Antiviral State by One Intranasal Dose of eTIP1 μLipoplexes Protects Animals from SARS-CoV-2 Infection and Pathology

This Example describes the results of experiments illustrating that induction of an antiviral state by one intranasal dose of eTIP1 μlipoplexes protects animals from SARS-CoV-2 infection and pathology.

The finding that the eTIP1 DVG interferes with viral infection by systemically inducing host antiviral responses raises its possible application as an antiviral treatment. However, practical considerations may limit applicability of eTIP1 particles, which require the PV receptor (PVR) to enter target cells and may be neutralized by pre-existing PV1 immunity in vaccinated individuals. Therefore, additional experiments were performed to investigate if synthetic nanostructured lipid/RNA complexes (herein termed lipoplexes) that deliver RNA via universal endocytosis could provide a universal carrier for the eTIP1 DVG. RNA/lipoplexes are not affected by pre-existing immunity and may be administered repeatedly in multiple treatments. A cationic lipid formulation was chosen in this study, which binds well to the phosphate backbone of nucleic acids, can be prepared with relative ease, and has been extensively characterized (FIG. 10A). In addition, lipoplexes protect RNA molecules from hydrolysis and support their in-vivo delivery to mucosal reparatory surfaces.

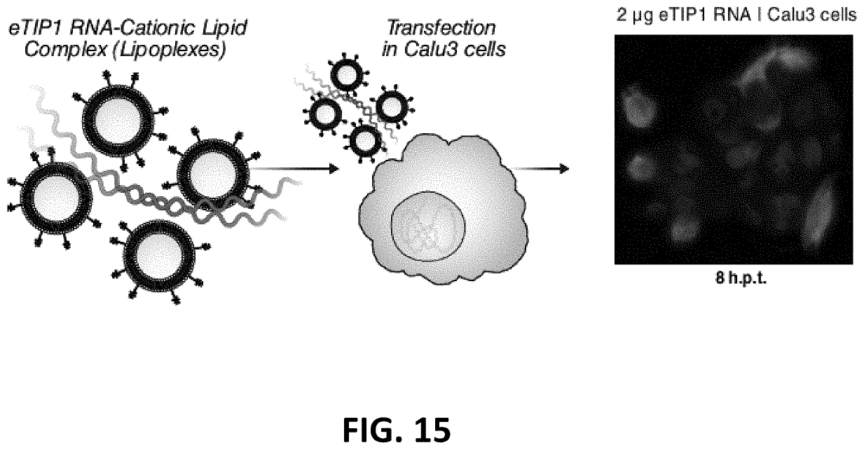

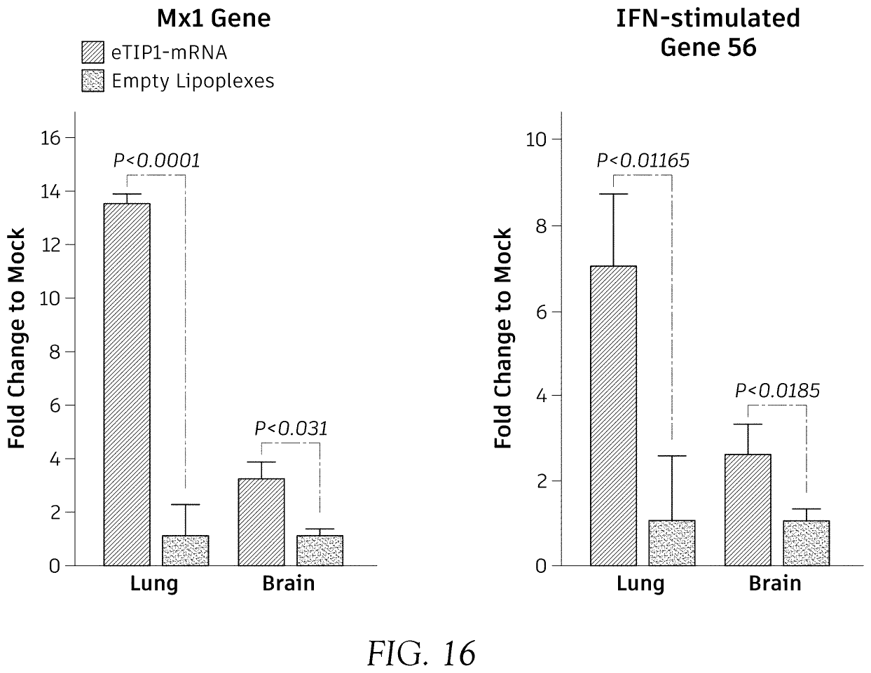

First, the potential of lipoplexes as delivery vehicles of synthetic eTIP1 RNA in cell culture was confirmed (see, e.g., FIG. 15). Next, their capacity to induce a protective antiviral response 20 h after a single intranasal administration was examined in K18-hACE2 mice susceptible to SARS-CoV-2 infection. As observed for eTIP1 particles (see, e.g., FIG. 9A), a single intranasal delivery of eTIP1 RNA lipoplexes, but not empty lipoplexes, potently induced IFN-responsive genes in the lungs (e.g., IFIT1, WIT 2, WIT 3, WIT 3b, IRF7, ISG15, Mx1, Mx2, Cxcl10, and OAS L2) (see, e.g., FIG. 10A). A mild IFN-response was also observed in brain tissues (FIG. 10A). These results were confirmed with RT-qPCR of mRNA from lung and brain of mice infected with eTIPi intranasally for 24 h. Two IFN-induced genes (ISGs), MX1 and ISG56 (IFIT1), were up-regulated in the eTIP1-treated group, compared with the mock control (see, e.g., FIG. 16). Thus, intranasal delivery of eTIPI RNA lipoplexes in mice stimulated innate antiviral immune responses, similar to what was observed with eTIP1 particles.

Next, if intranasal inoculation of eTIP1 μlipoplex or eTIP1 particles leads to replication and production of eTIP1-encoded proteins in different sites of the head and lungs of treated animals was investigated. At 24 h after inoculation, eTIP1 replication was examined in the head and lungs of inoculated animals by immunohistochemistry with an antibody against poliovirus 3B/VPg. In both cases, eTIP1 replication was observed only in a few epithelial cells within the ethmoid turbinates of the upper respiratory nasal cavity (see, e.g., FIGS. 10A-10B). Of note, no eTIP1 replication was observed in lungs (not shown), even though activation of ISGs, recruitment of eosinophils and plasmacytoid dendritic cells was observed (see, e.g., FIG. 9A-9B), and full protection from PV infection (see, e.g., FIG. 8B). In conclusion, intranasal inoculation with either lipoplex or particle eTIP1 μled to their restricted replication in the upper respiratory tract (see, e.g., FIG. 10B) but induced a non-cell-autonomous antiviral response that systemically protects the entire respiratory tract (see, e.g., FIG. 10C).

To better understand the mechanism of protection, additional experiments were performed to investigate if eTIPi replicates in the same cells and tissues as the pathogenic virus. Highly susceptible mice (IFNR^{-/-}) were inoculated intramuscularly with 200 PFU of PV1 in the presence or absence of eTIP1 (50,000 infectious units). Intramuscular inoculation ensures that PV1 and eTIP1 have a higher chance to co-infected cells and therefore eTIP1 can encapsidated by capsid proteins provided in trans by PV1. Virus and eTIP1 replication were examined in muscles, spleen and spinal cord by RT-qPCR. eTIP1 RNA accumulated at the site of inoculation on days 1, 3 and decayed by day 6 (see, e.g., FIG. 10D). In contrast, eTIP1 RNA was barely or not detected in either spleen or spinal cord, indicating that the eTIP1 does not spread beyond the site of inoculation even in the presence of PV1 (see, e.g., FIG. 10D). This result indicates that eTIP1 inhibits virus replication at distal sites (e.g., the central nervous system) even though the eTIP1 itself does not spread from the site of initial infection.

If eTIP1 protects animals from SARS-CoV-2 infection was investigated. A single intranasal dose of eTIP1 μlipoplex was applied to K18-hACE2 mice, and after 20 hours, the mice were infected intranasally with 6×10⁴ PFU SARS-CoV-2. Whether eTIPI treatment affects SARS-CoV-2 replication in relevant tissues, lungs and brains was measured, which were collected on days 3 and 6 post-infection. SARS-CoV-2 replication was determined by plaque assay and RT-qPCR (FIG. 11A), as well as immunohistochemistry analysis with antibodies against the nucleocapsid (NP) and spike (SP) proteins (see, e.g., FIGS. 11B-11C). As expected, control lipoplex-treated mice exhibited significant SARS-CoV-2 titers and widespread NP and SP immuno-reactivity throughout the lung and brain tissue (see, e.g., FIGS. 11A-11C). Strikingly, administration of the eTIP1 μlipoplex reduced SARS-CoV-2 titers and viral RNA accumulation by 2-3 μlogs in the lungs and brains (see, e.g., FIG. 11A), and immunohistochemistry confirmed that eTIP1 treatment strongly reduced SARS-CoV-2 replication in lungs and brain (see, e.g., FIGS. 11B-11C).

Weight loss is a sensitive measure of animal distress. SARS-CoV-2 infected mice lost weight due to COVID-19 disease progression. Strikingly, a single intra-nasal eTIP1 dose prevented abrogated weight loss in SARS-CoV-2 infected. In fact, eTIP1-treated animals maintained their body weight throughout the experiment, to the same extent of the mock-infected control group (see, e.g., FIG. 12A). This experiment indicates that in addition to blocking SARS-CoV-2 viral replication, the eTIP1 prevents disease symptoms; furthermore, it also confirms that eTIP1 itself does not cause distress.

Whether eTIP1 protects lungs from COVID-like inflammation damage was investigated. Lung sections were analyzed after staining with hematoxylin and eosin (H&E) and scored on tissue pathology. SARS-CoV-2-infected mice exhibited significantly higher histopathology scores than mock-infected mice (see, e.g., FIGS. 12B-12C). The major histopathology findings in infected mice were proteinaceous debris in the alveolar space, neutrophils in the interstitial space, and alveolar septal thickening. These observations are consistent with previous studies that detected signs of lung injury, including interstitial pneumonia, inflammatory cell infiltrates, and alveolar septal thickening. Histopathology analysis showed treatment with eTIP1 μlipoplexes reduced SARS-CoV-2 inflammation in the lung (histopathology score of 1/16 compared to empty-lipoplex histopathology score of 5.4/16) at day 3 after infection (see, e.g., FIGS. 12B-12C, SARS-CoV-2/eTIP1). Of note, even though SARS-CoV-2 replication was strongly inhibited, lymphoid cells infiltrated into the lung, which correlated with protection; this may be linked to an eTIP1-mediated antiviral protective environment in the lungs. Importantly, no peribronchial inflammation was observed in the lungs when animals were treated with eTIP1 alone, indicating that eTIP1 RNA levels clear over time at the site of infection and do not cause persistent inflammation (see, e.g., FIG. 12B, eTIP1). These experiments illustrate that a single intranasal eTIPI treatment reduces replication of SARS-CoV-2 by orders of magnitude, reduces lung inflammation in vivo, and alleviates the symptoms of COVID-19 disease.

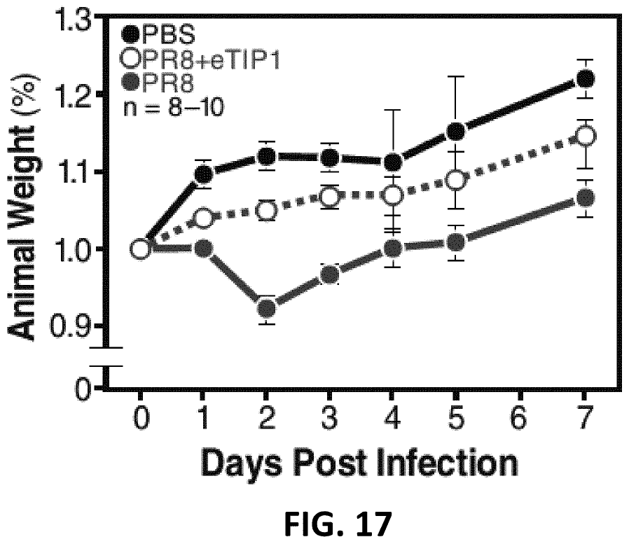

Further supporting the potential of eTIP1 as a broad-spectrum antiviral, following observation was made: a single intranasal inoculation with eTIPI protected mice from influenza virus infection and disease (FIG. 17). In conclusion, intranasal delivery of eTIP1 nanoparticles safely and potently stimulates host innate antiviral immune responses that protect from infection, reduce viral loads and prevent disease. Therefore, eTIP1 disclosed herein has compelling potential for clinical efficacy in the treatment of respiratory diseases.

### Example 26. eTIP Enhances Antibody Response to Nucleic Acid-Based Vaccines

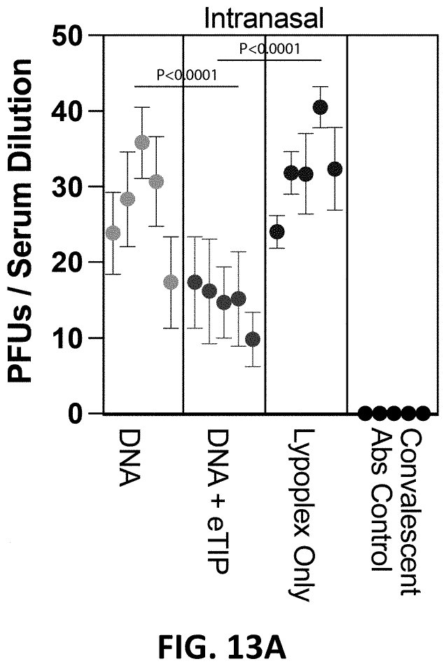

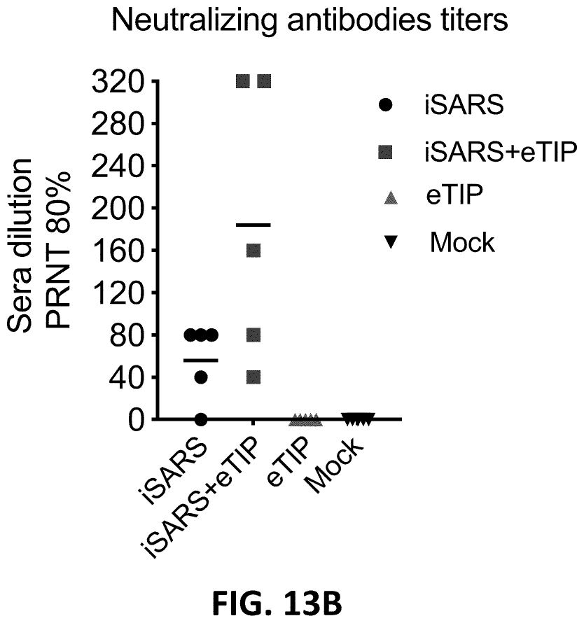

This Example describes the results of experiments illustrating that eTIP enhances antibody response to nucleic acid-based vaccines. In these experiments, mammalian expression vector encoding the pre-fusion stabilized spike protein of SARS-CoV-2 (S HexaPro) was formulated into nanostructured lipoplexes complexes. Mice bearing the human poliovirus receptor (Tg21) were vaccinated with 30 μg of S HexaPro DNA lipoplexes intranasally alone, or followed by intranasal delivery of 1.5×10⁶ biological eTIPs after 30 hours. Two weeks after vaccination, serial dilutions of blood sera from vaccinated animals (5 in each group) were analyzed for the presence of anti-SARS-CoV-2 neutralizing antibodies using plaque assay. Sera from mice vaccinated with a combination of DNA lipoplexes and biological eTIPs inhibited SARS-CoV-2 plaque formation (PFUs) more efficiently (2.5 folds) than sera of mice vaccinated with DNA lipoplexes alone. No PFUs were detected when similar dilutions of serum obtained from convalescent human patients vaccinated with two doses of Moderna vaccine RNA-1273 was used. Intranasal delivery of eTIP enhanced production of anti-SARS-CoV-2 neutralizing antibodies (FIG. 13A).

### Example 27. eTIP Enhances Antibody Response to SARS-CoV-2 Inactivated Vaccines

This Example describes the results of experiments illustrating that eTIP enhances antibody response to SARS-CoV-2 inactivated vaccines. Purified inactivated viruses have been traditionally used for vaccine development, and such vaccines have been found to be safe and effective for the prevention of diseases caused by viruses such as influenza virus and poliovirus. Since the outbreak began, researchers around the world have been trying to develop vaccines for COVID-19. Efforts towards the development of a vaccine have led to several candidate vaccines, derived from multiple platforms, including inactivated vaccines. For example, CoronaVac (Sinovac) has shown good immunogenicity, which supported it use in humans.

The experiments described herein tested whether eTIP can enhance immunogenicity elicit by inactivated and purified SARS CoV2 vaccine, which may reduce the amount of vaccine required to induce neutralizing antibodies against SARS CoV2 and extend vaccine protection against COVID19. Mice were immunized with 5 μg of inactivated SARS-CoV-2 particles (iSARS) intramuscularly, together with eTIP (5×10⁶ infectious units). As control, mice were inoculated with iSARS alone, eTIP, or saline (mock). After two inoculations (week 0 and 5), an 80% plaque reduction neutralizing test (PRNT 80%) was carried out on Vero-E6 cells ten weeks after first inoculation. Results demonstrate that eTIP increases production of neutralizing antibodies 5 folds (see, e.g., FIG. 13B).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1. Wimmer, E., Hellen, C. V. T. & Cao, X. GENETICS OF POLIOVIRUS. 1-84 (1993).
2. Abdool Karim, S. S., and de Oliveira, T. (2021). New SARS-CoV-2 Variants-Clinical, Public Health, and Vaccine Implications. N Engl J Med.
3. Ascough, S., Vlachantoni, I., Kalyan, M., Haijema, B.-J., Wallin-Weber, S., Dijkstra-Tiekstra, M., Ahmed, M. S., van Roosmalen, M., Grimaldi, R., Zhang, Q., et al. (2019). Local and Systemic Immunity against Respiratory Syncytial Virus Induced by a Novel Intranasal Vaccine. A Randomized, Double-Blind, Placebo-controlled Clinical Trial. Am J Respir Crit Care Med 200, 481-492.
4. Brooke, C. B., Ince, W. L., Wei, J., Bennink, J. R., and Yewdell, J. W. (2014). Influenza A virus nucleoprotein selectively decreases neuraminidase gene-segment packaging while enhancing viral fitness and transmissibility. Proc Natl Acad Sci USA 111, 16854-16859.
5. Burrill, C. P., Strings, V. R., and Andino, R. (2013a). Poliovirus: Generation, Quantification, Propagation, Purification, and Storage. Current Protocols in Microbiology 29, 15H.1.1-15H.1.27.
6. Burrill, C. P., Strings, V. R., Schulte, M. B., and Andino, R. (2013b). Poliovirus: Generation and Characterization of Mutants. Current Protocols in Microbiology 29, 15H.2.1-15H.2.32.
7. Caskey, M., Lefebvre, F., Filali-Mouhim, A., Cameron, M. J., Goulet, J.-P., Haddad, E. K., Breton, G., Trumpfheller, C., Pollak, S., Shimeliovich, I., et al. (2011). Synthetic double-stranded RNA induces innate immune responses similar to a live viral vaccine in humans. J Exp Med 208, 2357-2366.
8. Channappanavar, R., Fehr, A. R., Vijay, R., Mack, M., Zhao, J., Meyerholz, D. K., and Perlman, S. (2016). Dysregulated Type I Interferon and Inflammatory Monocyte-Macrophage Responses Cause Lethal Pneumonia in SARS-CoV-Infected Mice. Cell Host & Microbe 19, 181-193.
9. Crotty, S. (2004). Poliovirus vaccine strains as mucosal vaccine vectors and their potential use to develop an AIDS vaccine. Advanced Drug Delivery Reviews 56, 835-852.
10. Deng, X., Garcia-Knight, M. A., Khalid, M. M., Servellita, V., Wang, C., Morris, M. K., Sotomayor-Gonzalez, A., Glasner, D. R., Reyes, K. R., Gliwa, A. S., et al. (2021). Transmission, infectivity, and antibody neutralization of an emerging SARS-CoV-2 variant in California carrying a L452R spike protein mutation. medRxiv.
11. Dinnon, K. H., Leist, S. R., Schafer, A., Edwards, C. E., Martinez, D. R., Montgomery, S. A., West, A., Yount, B. L., Hou, Y. J., Adams, L. E., et al. (2020). A mouse-adapted model of SARS-CoV-2 to test COVID-19 countermeasures. Nature 1-16.
12. Gu, H., Chen, Q., Yang, G., He, L., Fan, H., Deng, Y.-Q., Wang, Y., Teng, Y., Zhao, Z., Cui, Y., et al. (2020). Rapid adaptation of SARS-CoV-2 in BALB/c mice: Novel mouse model for vaccine efficacy. 1-38.
13. Huang, A. S., and Baltimore, D. (1970). Defective Viral Particles and Viral Disease Processes. Nature 1-3.
14. Ida-Hosonuma, M., Iwasaki, T., Yoshikawa, T., Nagata, N., Sato, Y., Sata, T., Yoneyama, M., Fujita, T., Taya, C., Yonekawa, H., et al. (2005). The Alpha/Beta Interferon Response Controls Tissue Tropism and Pathogenicity of Poliovirus. Journal of Virology 79, 4460-4469.
15. Jangra, S., De Vrieze, J., Choi, A., Rathnasinghe, R., Laghlali, G., Uvyn, A., Van Herck, S., Nuhn, L., Deswarte, K., Zhong, Z., et al. (2021). Sterilizing Immunity against SARS-CoV-2 Infection in Mice by a Single-Shot and Lipid Amphiphile Imidazoquinoline TLR7/8 Agonist-Adjuvanted Recombinant Spike Protein Vaccine*. Angew Chem Int Ed Engl 60, 9467-9473.
16. Kikkert, M. (2020). Innate Immune Evasion by Human Respiratory RNA Viruses. J Innate Immun 12, 4-20.
17. Kitamura, N., Semler, B. L., Rothberg, P. G., Larsen, G. R., and Wimmer, E. (1981). Primary structure, gene organization and polypeptide expression of poliovirus. 1-7.
18. Kranz, L. M., Diken, M., Haas, H., Kreiter, S., Loquai, C., Reuter, K. C., Meng, M., Fritz, D., Vascotto, F., Hefesha, H., et al. (2016). Systemic RNA delivery to dendritic cells exploits antiviral defence for cancer immunotherapy. Nature 534, 396-401.
19. Meganck, R. M., and Baric, R. S. (2021). Developing therapeutic approaches for twenty-first-century emerging infectious viral diseases. Nat Med 1-10.
20. Meyerholz, D. K., and Beck, A. P. (2019). Histopathologic Evaluation and Scoring of Viral Lung Infection. In Hepatitis C Virus Protocols, (New York, N.Y.: Springer US), pp. 205-220.
21. Moreau, G. B., Burgess, S. L., Sturek, J. M., Donlan, A. N., Petri, W. A., Jr., and Mann, B. J. (2020). Evaluation of K18- hACE2mice as a model of SARS-CoV-2 infection. 1-12.
22. Myong, S., Cui, S., Cornish, P. V., Kirchhofer, A., Gack, M. U., Jung, J. U., Hopfner, K.-P., and Ha, T. (2009). Cytosolic viral sensor RIG-I is a 5′-triphosphate-dependent translocase on double-stranded RNA. Science 323, 1070-1074.
23. Nagata, N., Iwasaki, T., Ami, Y., Sato, Y., Hatano, I., Harashima, A., Suzaki, Y., Yoshii, T., Hashikawa, T., Sata, T., et al. (2004). A poliomyelitis model through mucosal infection in transgenic mice bearing human poliovirus receptor, TgPVR21. Virology 321, 87-100.
24. Nelemans, T., and Kikkert, M. (2019). Viral Innate Immune Evasion and the Pathogenesis of Emerging RNA Virus Infections. Viruses 11, 961.
25. Ohka, S., Igarashi, H., Nagata, N., Sakai, M., Koike, S., Nochi, T., Kiyono, H., and Nomoto, A. (2007). Establishment of a Poliovirus Oral Infection System in Human Poliovirus Receptor-Expressing Transgenic Mice That Are Deficient in Alpha/Beta Interferon Receptor. Journal of Virology 81, 7902-7912.
26. Pathinayake, P., Hsu, A., and Wark, P. (2015). Innate Immunity and Immune Evasion by Enterovirus 71. Viruses 7, 6613-6630.
27. Peersen, O. B. (2017). Picornaviral polymerase structure, function, and fidelity modulation. Virus Research 234, 4-20.
28. Perrault, J., and Semler, B. L. (1979). Internal genome deletions in two distinct classes of defective interfering particles of vesicular stomatitis virus. Proc Natl Acad Sci USA 76, 6191-6195.
29. Perrault, J. (1981). Origin and Replication of Defective Interfering Particles. In Initiation Signals in Viral Gene Expression, A. J. Shatkin, ed. (Berlin, Heidelberg: Springer Berlin Heidelberg), pp. 151-207.
30. Pfeiffer, J. K. (2010). Innate Host Barriers to Viral Trafficking and Population Diversity. (Elsevier), pp. 85-118.
31. Rahim, M. M. A., Parsons, B. D., Price, E. L., Slaine, P. D., Chilvers, B. L., Seaton, G. S., Wight, A., Medina-Luna, D., Dey, S., Grandy, S. L., et al. (2020). Defective Influenza A Virus RNA Products Mediate MAVS-Dependent Upregulation of Human Leukocyte Antigen Class I Proteins. Journal of Virology 94.
32. Rezelj, V. V., Carrau, L., Merwaiss, F., Levi, L. I., Erazo, D., Tran, Q. D., Henrion-Lacritick, A., Gausson, V., Suzuki, Y., Shengjuler, D., et al. (2021). Defective viral genomes as therapeutic interfering particles against flavivirus infection in mammalian and mosquito hosts. Nature Communications 1-14.
33. Rogers, T. F., Zhao, F., Huang, D., Beutler, N., Burns, A., He, W.-T., Limbo, O., Smith, C., Song, G., Woehl, J., et al. (2020). Isolation of potent SARS-CoV-2 neutralizing antibodies and protection from disease in a small animal model. Science 369, 956-963.
34. Scheideler, M., Vidakovic, I., and Prassl, R. (2020). Lipid nanocarriers for microRNA delivery. Chem Phys Lipids 226, 104837.
35. Schlee, M., Roth, A., Hornung, V., Hagmann, C. A., Wimmenauer, V., Barchet, W., Coch, C., Janke, M., Mihailovic, A., Wardle, G., et al. (2009). Recognition of 5′ triphosphate by RIG-I helicase requires short blunt double-stranded RNA as contained in panhandle of negative-strand virus. Immunity 31, 25-34.
36. Schmolke, M., and Garcia-Sastre, A. (2010). Evasion of innate and adaptive immune responses by influenza A virus. Cell Microbiol 12, 873-880.
37. Schoggins, J. W., and Rice, C. M. (2011). Interferon-stimulated genes and their antiviral effector functions. Current Opinion in Virology 1, 519-525.
38. Schoggins, J. W., MacDuff, D. A., Imanaka, N., Gainey, M. D., Shrestha, B., Eitson, J. L., Mar, K. B., Richardson, R. B., Ratushny, A. V., Litvak, V., et al. (2014). Pan-viral specificity of IFN-induced genes reveals new roles for cGAS in innate immunity. Nature 505, 691-695.
39. Semler, B. L., Perrault, J., Abelson, J., and Holland, J. J. (1978). Sequence of a RNA templated by the 3′-OH RNA terminus of defective interfering particles of vesicular stomatitis virus. Proc Natl Acad Sci USA 75, 4704-4708.
40. Shirogane, Y., Rousseau, E., Voznica, J., Xiao, Y., Su, W., Catching, A., Whitfield, Z. J., Rouzine, I. M., Bianco, S., and Andino, R. (2021). Experimental and mathematical insights on the interactions between poliovirus and a defective interfering genome. bioRxiv 7, 118-150.
41. Sun, Y., Jain, D., Koziol-White, C. J., Genoyer, E., Gilbert, M., Tapia, K., Panettieri, R. A., Hodinka, R. L., and L6pez, C. B. (2015). Immunostimulatory Defective Viral Genomes from Respiratory Syncytial Virus Promote a Strong Innate Antiviral Response during Infection in Mice and Humans. PLoS Pathog 11, e1005122-21.
42. Trapnell, C., Roberts, A., Goff, L., Pertea, G., Kim, D., Kelley, D. R., Pimentel, H., Salzberg, S. L., Rinn, J. L., and Pachter, L. (2012). Differential gene and transcript expression analysis of RNA-seq experiments with TopHat and Cufflinks. Nature Protocols 7, 562-578.
43. Vignuzzi, M., and L6pez, C. B. (2019a). Defective viral genomes are key drivers of the virus-host interaction. Nat Microbiol 1-13.
44. Vignuzzi, M., and L6pez, C. B. (2019b). Defective viral genomes are key drivers of the virus-host interaction. Nat Microbiol 1-13.
45. Wang, C., Li, W., Drabek, D., Okba, N. M. A., Haperen, R., Osterhaus, A. D. M. E., Kuppeveld, F. J. M., Haagmans, B. L., Grosveld, F., and Bosch, B.-J. (2020). A human monoclonal antibody blocking SARS-CoV-2 infection. Nature Communications 1-6.
46. White, K. M., Rosales, R., Yildiz, S., Kehrer, T., Miorin, L., Moreno, E., Jangra, S., Uccellini, M. B., Rathnasinghe, R., Coughlan, L., et al. (2021). Plitidepsin has potent preclinical efficacy against SARS-CoV-2 by targeting the host protein eEF1A. Science 371, 926-931.
47. WHO (2021). COVID-19 Weekly Epidemiological Update. 1-33.
48. Winkler, E. S., Bailey, A. L., Kafai, N. M., Nair, S., McCune, B. T., Yu, J., Fox, J. M., Chen, R. E., Earnest, J. T., Keeler, S. P., et al. (2020). SARS-CoV-2 infection of human ACE2-transgenic mice causes severe lung inflammation and impaired function. Nature Immunology 1-21.
49. Xiao, Y., Dolan, P. T., Goldstein, E. F., Li, M., Farkov, M., Brodsky, L., and Andino, R. (2017). Poliovirus intrahost evolution is required to overcome tissue-specific innate immune responses. Nature Communications 1-12.
50. Zhou, B., Thao, T. T. N., Hoffmann, D., Taddeo, A., Ebert, N., Labroussaa, F., Pohlmann, A., King, J., Steiner, S., Kelly, J. N., et al. (2021). SARS-CoV-2 spike D614G change enhances replication and transmission. Nature 592, 122-127.
51. Zhu, N., Zhang, D., Wang, W., Li, X., Yang, B., Song, J., Zhao, X., Huang, B., Shi, W., Lu, R., et al. (2020). A Novel Coronavirus from Patients with Pneumonia in China, 2019. N Engl J Med NEJMoa2001O17-NEJMoa2001O17.

What is claimed is:

1. A nucleic acid construct comprising a nucleic acid sequence encoding a modified enterovirus genome, wherein the modified enterovirus genome is devoid of at least a portion of the nucleic acid sequence encoding viral structural proteins.

2. The nucleic acid construct of claim 1, wherein the modified enterovirus genome is devoid of at least a portion of the sequence encoding VP1, VP2, VP3, VP4, or a combination of any thereof.

3. The nucleic acid construct of any one of claims 1 to 2, wherein the modified enterovirus genome or replicon RNA is devoid of a substantial portion of the nucleic acid sequence encoding viral structural proteins.

4. The nucleic acid construct of any one of claims 1 to 3, wherein the modified enterovirus genome comprises no nucleic acid sequence encoding viral structural proteins.

5. The nucleic acid construct of any one of claims 1 to 4, wherein the modified enterovirus genome is derived from a virus belonging to a Rhinovirus species selected from the group consisting of Rhinovirus A, Rhinovirus B, and Rhinovirus C.

6. The nucleic acid construct of any one of claims 1 to 4, wherein the modified enterovirus genome is derived from a virus belonging to an Enterovirus species selected from the group consisting of Enterovirus A, Enterovirus B, Enterovirus C, Enterovirus D, Enterovirus E, Enterovirus F, Enterovirus G, Enterovirus H, Enterovirus I, Enterovirus J, Enterovirus K, and Enterovirus L.

7. The nucleic acid construct of claim 6, wherein the modified enterovirus genome is derived from a poliovirus of the Enterovirus C species.

8. The nucleic acid construct of claim 7, wherein the modified enterovirus genome is derived from a poliovirus serotype selected from the group consisting of PV1, PV2, and PV3.

9. The nucleic acid construct of claim 8, wherein the modified poliovirus genome or replicon RNA is derived from poliovirus type 1 (PV1).

10. The nucleic acid construct of any one of claims 1 to 9, wherein the nucleic acid sequence encoding the modified poliovirus genome has at least 80%, at least 90%, at least 95%, at least 96%, at least 97%, at least 98%, at least 99%, or 100% sequence identity to the nucleic acid sequence of SEQ ID NO: 1.

11. The nucleic acid construct of any one of claims 1 to 10, wherein the nucleic acid sequence encoding a modified poliovirus genome is operably linked to a heterologous nucleic acid sequence.

12. The nucleic acid construct of claim 11, wherein the heterologous nucleic acid sequence comprises a promoter sequence or a coding sequence for a selectable marker.

13. The nucleic acid construct of any one of claims 1 to 12, wherein the nucleic acid sequence encoding a modified poliovirus genome is incorporated into an expression cassette or an expression vector.

14. A defective interfering (DI) particle of enterovirus comprising a nucleic acid construct of any one of claims 1 to 13.

15. The DI particle of claim 14, wherein the nucleic acid construct is encapsidated by heterologous capsid structural proteins.

16. A recombinant cell comprising (a) a nucleic acid construct of any one of claims 1 to 13, and/or (b) a DI particle of any one of claims 14 to 15.

17. The recombinant cell of claim 16, wherein the recombinant cell is a eukaryotic cell.

18. The recombinant cell of claim 17, wherein the eukaryotic cell is an animal cell.

19. The recombinant cell of claim 18, wherein the animal cell is a human cell.

20. A method for producing a defective interfering (DI) particle of enterovirus comprising:
a) providing a host cell engineered to express enterovirus structural proteins;
b) transfecting the provided host cell with a nucleic acid construct according to any one of claims 1 to 13; and
c) culturing the transfected host cell under conditions for production of a DIP of enterovirus comprising the nucleic acid construct encapsidated by the expressed enterovirus structural proteins.

21. The method of claim 20, further harvesting the produced DI particle.

22. A defective interfering (DI) particle produced by the method of any one of claims 20-21.

23. A pharmaceutical composition comprising a pharmaceutically acceptable excipient and:
(a) a DI particle according to any one of claims 14-15 and 22;
(b) a nucleic acid construct according to any one of claims 1 to 13; and/or
(c) a recombinant cell according to any one of claims 16 to 19.

24. The pharmaceutical composition of claim 23, wherein the composition comprises a DI particle of any one of claims 14-15 and 16, and a pharmaceutically acceptable excipient.

25. The pharmaceutical composition of claim 23, wherein the composition comprises a nucleic acid construct of any one of claims 1 to 13, and a pharmaceutically acceptable excipient.

26. The pharmaceutical composition of any one of claims 23 to 25, wherein the composition is formulated in a liposome, a lipid nanoparticle, or a polymer nanoparticle.

27. The pharmaceutical composition of any one of claims 23 to 26, wherein the composition is an immunogenic composition.

28. The pharmaceutical composition of claim 27, wherein the immunogenic composition is formulated as a vaccine.

29. The pharmaceutical composition of any one of claims 23 to 28, wherein the pharmaceutical composition is formulated as an adjuvant.

30. The pharmaceutical composition of any one of claims 23 to 29, wherein the pharmaceutical composition is formulated for one or more of intranasal administration, transdermal administration, intraperitoneal administration, intramuscular administration, intravenous administration, and oral administration.

31. A method for eliciting an immune response in a subject in need thereof, the method comprises administering to the subject a composition comprising:
(a) a DI particle according to any one of claims 14-15 and 22;
(b) a nucleic acid construct according to any one of claims 1-13;
(c) a recombinant cell according to any one of claims 16 to 19; and/or
(d) a pharmaceutical composition according to any one of claims 23-30.

32. A method for preventing and/or treating a health condition in a subject in need thereof, the method comprises prophylactically or therapeutically administering to the subject a composition comprising:
(a) a DI particle according to any one of claims 14-15 and 22;
(b) a nucleic acid construct according to any one of claims 1-13;
(c) a recombinant cell according to any one of claims 16 to 19; and/or
(d) a pharmaceutical composition according to any one of claims 23-30.

33. The method of claim 32, wherein the condition is an immune disease or an infection.

34. The method of any one of claims 31 to 33, wherein the subject has or is suspected of having a condition associated with an immune disease or an infection.

35. The method of claim 34, wherein the infection is a seasonal respiratory viral infection or an acute respiratory viral infection.

36. The method of any one of claims 34 to 35, wherein the infection is caused by a virus belonging to a species of the Human orthopneumovirus genus, a species of the Enterovirus family, a species of the Coronaviridae family, or a subtype of the Orthomyxoviridae family.

37. The method of claim 36, wherein the orthomyxovirus is an influenza A virus or a Parainfluenza virus.

38. The method of claim 37, wherein the influenza A virus is selected from the group consisting of subtypes H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7.

39. The method of claim 37, wherein the parainfluenza virus is selected from the group consisting of subtypes HPIV-1, HPIV-2, HPIV-3, and HPIV-4.

40. The method of claim 36, wherein the coronavirus is β-CoV severe acute respiratory syndrome coronavirus (SARS-CoV), severe acute respiratory syndrome coronavirus 2 (SARS-CoV-2).

41. The method of claim 40, wherein the coronavirus β-CoV infection is associated with one or more subgenus Sarbecovirus selected from the group consisting of severe acute respiratory syndrome coronavirus SARSr-CoV (which includes all its strains such as SARS-CoV, SARS-CoV-2, and Bat SL-CoV-WIV1), subgenus Merbecovirus consisting of Tylonycteris bat coronavirus HKU4 (BtCoV-HKU4), Pipistrellus bat coronavirus HKU5 (BtCoV-HKU5), and Middle East respiratory syndrome-related coronavirus MERS-CoV (which includes the species HCoV-229E, HCoV-NL63, HCoV-OC43, HCoV-HKU1).

42. The method of claim 36, wherein the human orthomyxovirus is a human respiratory syncytial virus (HRSV).

43. The method of claim 42, wherein the HRSV is associated with subtype A and/or subtype B.

44. The method of claim 36, the viral infection is an enteroviral infection or a rhinoviral infection.

45. The method of claim 44, wherein the rhinoviral infection is associated with one or more Rhinovirus species selected from the group consisting of rhinovirus A species, rhinovirus B species, and rhinovirus C species.

46. The method of claim 44, wherein the enteroviral infection is associated with one or more Enterovirus species selected from the group consisting of Enterovirus A species, Enterovirus B species, Enterovirus C species, Enterovirus D species, Enterovirus E species, Enterovirus F species, Enterovirus G species, Enterovirus H species, Enterovirus I species, Enterovirus J species, Enterovirus K species, and Enterovirus L species.

47. The method of any one of claims 44 to 46, wherein the viral infection is associated with one or more of poliovirus type 1 (PV1), poliovirus type 3 (PV3), coxsackievirus A2, coxsackievirus A4, coxsackievirus A16, coxsackievirus B1, coxsackievirus B3 (CV-B3), coxsackievirus B6, Parechovirus (echovirus), enterovirus A71 (EV-A71), enterovirus D68 (EV-D68), rhinovirus HRV16, and rhinovirus HRV1B.

48. The method of any one of claims 31 to 47, wherein the composition is formulated for one or more of intranasal administration, transdermal administration, intramuscular administration, intravenous administration, intraperitoneal administration, oral administration, or intra-cranial administration.

49. The method of any one of claims 31 to 48, wherein the administered composition results in an increased production of interferon in the subject.

50. The method of any one of claims 32 to 49, wherein the composition is administered to the subject individually as a single therapy (monotherapy) or as a first therapy in combination with at least one additional therapies.

51. The method of claim 50, wherein the at least one additional therapies is selected from the group consisting of chemotherapy, radiotherapy, immunotherapy, hormonal therapy, toxin therapy, targeted therapy, and surgery.

52. A kit for eliciting an immune response, for the prevention, and/or for the treatment of a health condition or a viral infection, the kit comprising:
(a) a DI particle according to any one of claims 14-15 and 22;
(b) a nucleic acid construct according to any one of claims 1-13;
(c) a recombinant cell according to any one of claims 16-19; and/or
(d) a pharmaceutical composition according to any one of claims 23-30.
